(19) United States
(12) Patent Application Publication
(10) Pub. No.: US 2022/0280548 A1
(43) Pub. Date: Sep. 8, 2022

(54) COMBINATION THERAPY FOR SPINAL MUSCULAR ATROPHY

(71) Applicant: Biogen MA Inc., Cambridge, MA (US)

(72) Inventor: Alexander McCampbell, Andover, MA (US)

(73) Assignee: Biogen MA Inc., Cambridge, MA (US)

(21) Appl. No.: 17/635,363

(22) PCT Filed: Aug. 14, 2020
(86) PCT No.: PCT/US2020/046546

§ 371 Date: Feb. 14, 2022

Related U.S. Application Data
(60) Provisional application No. 62/887,579, filed on Aug. 15, 2019.

(51) Int. Cl. A61K 31/7125; A61K 48/00; A61P 21/00; C12N 15/113

(52) U.S. Cl. CPC A61K 31/7125; A61K 48/005; A61P 21/00; C12N 15/113

(57) ABSTRACT

Aspects of the application relate to compositions and methods for treating spinal muscular atrophy in a subject. In particular, this application provides therapeutic combinations of a small molecule that promotes SMN function and/or a recombinant nucleic acid that encodes the survival of motor neuron 1 (SMN1) protein (e.g., in a viral vector), and/or an antisense oligonucleotide (ASO) that increases full-length survival of motor neuron 2 (SMN2) mRNA (e.g., that is targeted to a nucleic acid molecule encoding the survival of motor neuron 2 (SMN2) and promotes the inclusion of exon 7 in SMN2 mRNA).

### CROSS-REFERENCE TO RELATED APPLICATIONS

This application claims priority under 35 U.S.C. § 119(e) to U.S. Provisional Patent Application No. 62/887,579, filed Aug. 15, 2019, which is hereby incorporated by reference in its entirety.

### FIELD

The present application relates to methods and compositions for treating spinal muscular atrophy (SMA).

### BACKGROUND

Spinal muscular atrophy (SMA) is a neuromuscular disease caused by mutations or deletions in telomeric SMN1, a gene encoding a ubiquitously expressed protein (survival of motor neuron—SMN) involved in spliceosome biogenesis.

The SMN gene product is intracellular and SMN deficiency results in selective toxicity to lower motor neurons, resulting in progressive neuron loss and muscle weakness. The severity of the disease is modified by the copy number of a centromeric duplication of the homologous gene (SMN2), which carries a splice site mutation that results in production of only small amounts of the full length SMN transcript. Patients who carry one to two copies of SMN2 present with the severe form of SMA, characterized by onset in the first few months of life and rapid progression to respiratory failure. Patients with three copies of SMN2 generally exhibit an attenuated form of the disease, typically presenting after six months of age. Though many never gain the ability to walk, they rarely progress to respiratory failure, and often live into adulthood. Patients with four SMN2 copies may not present until adulthood with gradual onset of muscle weakness.

Although several therapies for SMA have been developed, there remains a need for treatments that increase intracellular SMN activity in motor neurons involved in spinal muscular atrophy for patients having different levels of disease severity.

### SUMMARY

In some aspects, the present application relates to a method of treating spinal muscular atrophy (SMA) in a subject having SMA comprising administering to the subject: a) a small molecule that increases SMN function, and b) a recombinant nucleic acid that encodes the survival of motor neuron 1 (SMN1) protein.

In some aspects, the present application relates to a method of treating spinal muscular atrophy (SMA) in a subject having SMA comprising administering to the subject: a) a small molecule that increases SMN function, and b) an antisense oligonucleotide (ASO) that increases full-length survival of motor neuron 2 (SMN2) mRNA.

In some aspects, the present application relates to a method of treating spinal muscular atrophy (SMA) in a subject having SMA comprising administering to the subject: a) a small molecule that increases SMN function, b) a recombinant nucleic acid that encodes the survival of motor neuron 1 (SMN1) protein, and c) an antisense oligonucleotide (ASO) that increases full-length survival of motor neuron 2 (SMN2) mRNA.

In some aspects, the present application relates to a combination therapy for spinal muscular atrophy (SMA) that involves administration (e.g., concurrently or sequentially), to a subject having SMA, of a small molecule that increases SMN function; and a recombinant nucleic acid encoding Survival motor neuron 1 (SMN1) and/or an oligomeric compound that increases full-length Survival motor neuron 2 (SMN2) mRNA. In some aspects, a small molecule that increases SMN function is a small molecule that increases full-length SMN2 mRNA in subject. In some aspects, a recombinant nucleic acid encoding SMN1 is provided in a viral vector, for example in a recombinant adeno-associated virus (rAAV). In some aspects, an oligomeric compound is an antisense oligonucleotide (ASO) that increases full-length SMN2 mRNA in a subject (e.g., by modulating SMN2 pre-mRNA splicing to increase the inclusion of exon 7 in SMN2 mRNA).

In some aspects, the present application relates to combination therapy for spinal muscular atrophy (SMA) that involves administration (e.g., concurrently or sequentially), to a subject having SMA, of a small molecule that increases SMN function; and a recombinant nucleic acid encoding Survival motor neuron 1 (SMN1) and/or an oligomeric compound that modulates exon-skipping (e.g., promotes exon 7 inclusion) in a nucleic acid encoding Survival motor neuron 2 (SMN2) mRNA. In some aspects, a small molecule that increases SMN function is a small molecule that increases full-length SMN2 mRNA in subject. In some aspects, a recombinant nucleic acid encoding SMN1 is provided in a viral vector, for example in a recombinant adeno-associated virus (rAAV). In some aspects, an oligomeric compound that induces exon-skipping in a nucleic acid encoding SMN2 is an antisense oligonucleotide (ASO) that modulates exon-skipping (e.g., promotes exon 7 inclusion) in SMN2 pre-mRNA.

In some aspects, the small molecule that increases SMN function is a splice modulator, an HDAC inhibitor, or a molecule that modulates the activity of an mRNA decapping enzyme. In some aspects, the small molecule is a splice modulator. In some aspects, the splice modulator is a SMN2 splice modulator. In some aspects, the splice modulator is a 7-Disubstituted-phenyl tetracycline. In some aspects, the splice modulator is a substituted isoindolinone. In some aspects, the splice modulator is a substituted carbazole derivative. In some aspects, the SMN2 splice modulators are substituted 1, 4-diazepanes. In some aspects, the SMN2 splice modulators are substituted pyridazines. In some aspects, the SMN2 splice modulator is Risdiplam. In some aspects, the SMN2 splice modulator is Branaplam.

In some aspects, the small molecule that increases SMN function (e.g., Risdiplam or Branaplam) and the recombinant nucleic acid (e.g., in a viral vector such as an rAAV) and/or the SMN2 ASO (e.g., nusinersen) are provided as separate compositions, but administered to a subject concurrently (e.g., at the same time or contemporaneously, for example during the same medical visit, for example during the same hour or day). In some aspects, the small molecule that increases SMN function (e.g., Risdiplam or Branaplam) and the recombinant nucleic acid (e.g., in a viral vector such as an rAAV) and/or SMN2 ASO (e.g., nusinersen) are provided as separate compositions, and administered to a subject sequentially during separate medical visits (for example, at different times, e.g., on different days) during a course of treatment (e.g., during a treatment regimen over a week, 2-4 weeks, a month, 1-12 months, a year, 2-5 years, or longer). In some aspects, the small molecule that increases SMN function (e.g., Risdiplam or Branaplam) is administered prior to and/or subsequent to the recombinant nucleic acid (e.g., an rAAV) and/or SMN2 ASO (e.g., nusinersen). In some aspects, the small molecule that increases SMN function (e.g., Risdiplam or Branaplam) and the recombinant nucleic acid (e.g., in a viral vector such as an rAAV) and/or SMN2 ASO (e.g., nusinersen) are administered at different frequencies (e.g., concurrently or sequentially). In some aspects, a subject is treated with a combination of a separate compositions that comprise either the small molecule that increases SMN function (e.g., Risdiplam or Branaplam), the recombinant nucleic acid (e.g., in a viral vector such as an rAAV), or the ASO, wherein the compositions are administered at different frequencies (e.g., concurrently or sequentially).

In some aspects, two or more different small molecules that increase SMN function (e.g., Risdiplam or Branaplam) are administered to a subject. In some aspects, two or more different recombinant SMN1 nucleic acids (e.g., in an rAAV) are administered to a subject. In some aspects, two or more different SMN2 ASOs are administered to a subject. In some aspects, different recombinant SMN1 nucleic acids (e.g., in an rAAV) and/or different SMN2 ASOs are administered to a subject during different medical visits.

Accordingly, in some aspects a method of treating SMA in a subject (e.g., a human subject) having SMA involves administering to the subject a small molecule that increases SMN function (e.g., Risdiplam or Branaplam); and a recombinant nucleic acid that encodes SMN1 (also referred to as a recombinant SMN1 gene) (e.g., in an rAAV) and/or a SMN2 ASO that increases full-length SMN2 mRNA in a subject (also referred to as a SMN2 ASO). In some aspects, a method of treating SMA in a subject comprises administering an effective amount of a small molecule that increases SMN function (e.g., Risdiplam or Branaplam) and a recombinant SMN1 gene (e.g., in an rAAV) and/or a SMN2 ASO (e.g., nusinersen) to a subject having SMA.

In some aspects, a subject having SMA has one or more symptoms of SMA (e.g., atrophy of the limb muscles, difficulty or inability walking, difficulty breathing, or other symptom of SMA). In some aspects, a subject having SMA has two mutant alleles of the genomic SMN1 gene. In some aspects, the subject has a deletion or mutation (e.g., loss of function point mutation) in each SMN1 allele. In some aspects, the subject is homozygous for a SMN1 gene mutation. In some aspects, the subject is heterozygous for two different SMN1 gene mutations.

In some aspects, the subject is a human subject. In some aspects, the subject is selected from the pediatric and adult population. In some aspects, the subject is greater than or equal to 18 years of age (e.g., 18 years of age or older). In some aspects, the subject is younger than 18 years of age, younger than 10 years of age, or younger than 6 years of age. In some aspects, the subject is around 2 weeks, 1 month, 3 months, 6 months, 1 year, 2 years, 3 years, 4 years, or 5 years of age.

In some aspects, the recombinant SMN1 gene is operatively linked to a promoter. In some aspects, the SMN1 gene is a human SMN1 gene. In some aspects, the SMN1 gene is codon optimized (e.g., for expression in humans). In some aspects, the recombinant nucleic acid encoding the SMN1 gene is a recombinant AAV genome comprising flanking AAV inverted terminal repeats (ITRs). In some aspects, the recombinant nucleic acid is administered within an AAV particle. In some aspects, the AAV particle comprises AAV capsid proteins (e.g., AAV9, AAVrh10, AAV8 capsid proteins). In some aspects, the AAV particle comprises AAVhu68 capsid proteins. In some aspects, the AAV particle comprises AAV9 capsid proteins.

In some aspects, the SMN2 ASO alters the splicing pattern of survival of motor neuron 2 (SMN2) pre-mRNA. In some aspects, the SMN2 ASO promotes the inclusion of exon 7 in survival of motor neuron 2 (SMN2) mRNA. In some aspects, the SMN2 ASO comprises a sequence complementary to intron 6 or intron 7 of a nucleic acid (e.g., SMN2 gene or SMN2 pre-mRNA) molecule encoding the SMN2 protein. In some aspects, the SMN2 ASO comprises a sequence complementary to intron 6 of a nucleic acid molecule (e.g., SMN2 gene or SMN2 pre-mRNA) encoding SMN2 protein. In some aspects, the SMN2 ASO comprises a sequence complementary to intron 7 of a nucleic acid molecule (e.g., SMN2 gene or SMN2 pre-mRNA) encoding SMN2 protein. In some aspects, the SMN2 ASO (e.g., nusinersen) comprises a sequence of SEQ ID NOs: 1, 25, or 26. In some aspects, the ASO is nusinersen. In some aspects, the SMN2 ASO (e.g., nusinersen) comprises one or more nucleobase or backbone modifications.

In some aspects, a recombinant SMN1 gene (e.g., in a viral vector) is administered (e.g., one or more times) to a subject previously treated with a small molecule that increases SMN function and/or a SMN2 ASO (e.g., nusinersen) therapy. In some aspects, a recombinant SMN1 gene (e.g., in a viral vector such as an rAAV) is administered (e.g., one or more times) to a subject undergoing a current treatment with a small molecule that increases SMN function (e.g., Risdiplam or Branaplam) and/or a SMN2 ASO (e.g., nusinersen) therapy. In some aspects, a therapy comprising a concurrent or sequential administration of a small molecule that increases SMN function (e.g., Risdiplam or Branaplam), and a) a recombinant SMN1 gene (e.g., in a viral vector such as an rAAV) and/or b) a SMN2 ASO (e.g., nusinersen) is initiated for a subject.

In some aspects, a small molecule that increases SMN function (e.g., Risdiplam or Branaplam) and a) an rAAV comprising a recombinant SMN1 gene (also referred to as an SMN1 rAAV) and/or b) the SMN2 ASO (e.g., nusinersen) are administered simultaneously. In some aspects, the small molecule that increases SMN function (e.g., Risdiplam or Branaplam) and the SMN1 rAAV and/or SMN2 ASO are administered concurrently. In some aspects, the small molecule that increases SMN function (e.g., Risdiplam or Branaplam) and the SMN1 rAAV and/or SMN2 ASO (e.g., nusinersen) are administered separately in different compositions. In some aspects, the small molecule that increases SMN function (e.g., Risdiplam or Branaplam) and the SMN1 rAAV and/or the SMN2 ASO (e.g., nusinersen) are administered sequentially. In some aspects, the small molecule that increases SMN function (e.g., Risdiplam or Branaplam) and the SMN1 rAAV and/or SMN2 ASO (e.g., nusinersen) are administered at different frequencies. In some aspects, the small molecule that increases SMN function (e.g., Risdiplam or Branaplam) is administered 1-6 times per year or more frequently (e.g., weekly or 2-4 times per month). In some aspects, the SMN1 rAAV is administered once. In some aspects, the SMN2 ASO is administered 1-6 times per year. In some aspects, two or more subsequent doses of the small molecule that increases SMN function (e.g., Risdiplam or Branaplam) alone and/or with the SMN2 ASO (e.g., nusinersen) are administered following an initial administration of the SMN1 rAAV and the SMN2 ASO (e.g., nusinersen). In some aspects, a subject receives one or more additional doses of SMN1 rAAV. In some aspects, first and second administrations of SMN1 rAAV are provided to a subject more than 6 months apart or more than 1 year apart. In some aspects, first and second SMN1 rAAV compositions comprise the same rAAV capsid protein. In some aspects, first and second SMN1 rAAV compositions comprise different rAAV capsid proteins.

In some aspects, the SMN1 rAAV is administered at a dose from 1×10¹⁰ to 5×10¹⁴ GC. In some aspects, the SMN1 rAAV is administered at a dose from 2×10¹⁰ to 2×10¹⁴ GC. In some aspects, the SMN1 rAAV is administered at a dose from 3×10¹³ to 5×10¹⁴ GC. In some aspects, the SMN1 rAAV is administered at a dose of 2×10¹⁴ GC.

In some aspects, a total of 5 mg to 60 mg per dose of SMN2 ASO is administered to the subject. In some aspects, a total of 5 mg to 20 mg per dose of SMN2 ASO is administered to the subject. In some aspects, a total of 12 mg to 50 mg per dose of SMN2 ASO is administered to the subject. In some aspects, a total of 12 mg to 48 mg per dose of SMN2 ASO is administered to the subject. In some aspects, a total of 12 mg to 36 mg per dose of SMN2 ASO is administered to the subject. In some aspects, a total of 28 mg per dose of SMN2 ASO is administered to the subject. In some aspects, a total of 12 mg per dose of SMN2 ASO is administered to the subject. In some aspects, the dose volume is 5 mL.

In some aspects the small molecule is administered via a suitable route (e.g., orally) and the rAAV and/or SMN2 ASO are administered (e.g., via injection or infusion) independently via a route that is suitable for the treatment(s), for example via an intrathecal, intracisternal magna space, intravenous, or intramuscular administration. In some aspects, the small molecule that increases SMN function (e.g., Risdiplam or Branaplam), SMN1 rAAV, and/or SMN2 ASO (e.g., nusinersen) are administered into the intrathecal space of the subject. In some aspects, the small molecule that increases SMN function (e.g., Risdiplam or Branaplam), SMN1 rAAV and/or SMN2 ASO (e.g., nusinersen) are administered into the intracisternal magna space of the subject. In some aspects, initial and/or subsequent doses of the small molecule that increases SMN function (e.g., Risdiplam or Branaplam), the recombinant SMN1 gene (e.g., in a rAAV), and/or SMN2 ASO (e.g., nusinersen) are administered intravenously or intramuscularly.

In some aspects, administration of the small molecule that increases SMN function (e.g., Risdiplam or Branaplam), and the SMN1 rAAV and/or SMN2 ASO (e.g., nusinersen) increase intracellular SMN protein level in the subject. In some aspects, SMN protein level is increased in the cervical, thoracic, and lumbar spinal cord segments of the subject (e.g., in motor neurons in the brain and/or spinal cord of the subject).

In some aspects, SMN protein expression in a subject having SMA is increased by administering to the subject (e.g., concurrently or sequentially) an effective amount of a small molecule that increases SMN function (e.g., Risdiplam or Branaplam) and an SMN1 rAAV and/or SMN2 ASO (e.g., nusinersen). In some aspects, the subject had previously been treated with a small molecule that increases SMN function (e.g., Risdiplam or Branaplam). In some aspects, the subject had previously been administered an SMN1 rAAV. In some aspects, the subject had previously been treated with a SMN2 ASO (e.g., nusinersen). In some aspects, SMN protein expression in a subject previously treated with an SMN1 rAAV is increased by administering an effective amount of a small molecule that increases SMN function (e.g., Risdiplam or Branaplam) and/or a SMN2 ASO (e.g., nusinersen) to the subject. In some aspects, SMN protein expression in a subject previously treated with a SMN2 ASO (e.g., nusinersen) is increased by administering an effective amount of a small molecule that increases SMN function (e.g., Risdiplam or Branaplam) and/or an SMN1 rAAV to the subject. In some aspects, SMN protein expression in a subject previously treated with a small molecule that increases SMN function (e.g., Risdiplam or Branaplam) is increased by administering an effective amount of a SMN1 rAAV and/or SMN2 ASO (e.g., nusinersen) to the subject.

In some aspects, a composition comprises a small molecule that increases SMN function (e.g., Risdiplam or Branaplam). In some aspects, a composition comprises a recombinant SMN1 gene (e.g., in a rAAV). In some aspects, a composition comprises a SMN2 ASO (e.g., nusinersen). In some aspects, a pharmaceutical composition described herein further comprises a pharmaceutically acceptable carrier. In some aspects, a therapeutically effective amount of the pharmaceutical composition is administered to a subject in need thereof. Any of the compositions described herein can be pharmaceutical compositions further comprising a pharmaceutically acceptable carrier. In some aspects, a pharmaceutical composition comprising a small molecule that increases SMN function (e.g., Risdiplam or Branaplam) is administered to the subject via any known route suitable for administering a small molecule drug (e.g., oral administration). In some aspects, a pharmaceutical composition comprising recombinant SMN1 gene is administered to the subject via any known route suitable for administering a recombinant SMN1 gene (e.g., via intravenous injection). In some aspects, a pharmaceutical composition comprising a SMN2 ASO (e.g., nusinersen) is administered to the subject via any known route suitable for administering an ASO (e.g., intrathecal injection). In some aspects, one or more of a small molecule that increases SMN function (e.g., Risdiplam or Branaplam), an SMN1 rAAV and/or SMN2 ASO (e.g., nusinersen) (e.g., as two or three separate compositions) are administered to a subject (e.g., a human subject) via an intrathecal route. In some aspects, one or more of a small molecule that increases SMN function (e.g., Risdiplam or Branaplam), an SMN1 rAAV and/or SMN2 ASO (e.g., as two or three separate compositions) are administered (e.g., via injection, infusion, using a pump and a catheter, or via other suitable technique) into the spinal canal, subarachnoid space, ventricular or lumbar CSF, by suboccipital puncture, or by other suitable route. In some aspects, one or more of a small molecule that increases SMN function (e.g., Risdiplam or Branaplam), a recombinant SMN1 gene (e.g., in a rAAV) and/or SMN2 ASO (e.g., as two or three separate compositions,) are administered to a subject (e.g., a human subject) via an intracranial, intraventricular, intracerebral, intraparenchymal, intravenous, or other suitable route. In some aspects, a small molecule that increases SMN function (e.g., Risdiplam or Branaplam) is administered to the subject via oral administration, while an SMN1 rAAV and/or SMN2 ASO (e.g., nusinersen) (e.g., as two or three separate compositions) are administered to a subject (e.g., a human subject) via injection (e.g., intravenous, intrathecal, intramuscular, intracranial, intraventricular, intracerebral, or intraparenchymal). In some aspects, a small molecule that increases SMN function (e.g., Risdiplam or Branaplam) is administered to the subject via oral administration, while an SMN1 rAAV and/or SMN2 ASO (e.g., nusinersen) (e.g., as two or three separate compositions) are administered (e.g., via injection, infusion, using a pump and a catheter, or via other suitable technique) into the spinal canal, subarachnoid space, ventricular or lumbar CSF, by suboccipital puncture, or by other suitable route. Whether administered concurrently or sequentially, each of the small molecule that increases SMN function (e.g., Risdiplam or Branaplam), SMN1 rAAV and SMN2 ASO (e.g., nusinersen) may be administered by any suitable or appropriate means known in the art (e.g., intrathecal, intravenous, etc.), and the small molecule that increases SMN function (e.g., Risdiplam or Branaplam), SMN1 rAAV and SMN2 ASO (e.g., nusinersen) may be administered by the same or by different means (e.g., via the same or different routes of administration).

In some aspects, a small molecule that increases SMN function (e.g., Risdiplam or Branaplam), SMN1 rAAV, and/or a SMN2 ASO (e.g., nusinersen) are used in the manufacture of a medicament (e.g., as two or three separate medicaments) for treating a disease or condition associated with Survival motor neuron protein (SMN), such as spinal muscular atrophy (SMA).

In some aspects, the present disclosure relates to a method of treating spinal muscular atrophy (SMA) in a subject having SMA, comprising administering an effective amount of a small molecule that increases SMN function (e.g., Risdiplam or Branaplam) and/or a recombinant SMN1 gene (e.g., in a rAAV) in separate compositions to a subject that was previously treated with an ASO that increases full-length SMN2 mRNA. In some aspects, the ASO treatment is discontinued and the small molecule and/or recombinant SMN1 gene can be provided as a replacement therapy. In some aspects, the ASO treatment is continued and the small molecule and/or recombinant SMN1 gene can be provided as an additional therapy (e.g., as an adjunct therapy).

In some aspects, the present disclosure relates to a method of treating spinal muscular atrophy (SMA) in a subject having SMA, comprising administering an effective amount of a small molecule that increases SMN function (e.g., Risdiplam or Branaplam) and/or an ASO that increases full-length SMN2 mRNA (e.g., nusinersen) in separate compositions to a subject that was previously administered a recombinant SMN1 gene (e.g., in a rAAV). In some aspects, the subject does not receive any additional recombinant SMN1 gene after administration of the small molecule and/or ASO is initiated. In some aspects, one or more additional doses of a recombinant SMN1 gene and/or the small molecule are administered after administration of the small molecule and/or ASO is initiated. In some aspects, the dosing schedule of one or more therapies can be maintained or changed when an additional therapy initiates. In some aspects, the dosing schedule of a recombinant SMN1 gene is maintained or changed after administration of the small molecule that increases SMN function and/or the SMN2 ASO initiates. In some aspects, the dosing schedule of a SMN2 ASO is maintained or changed after administration of the small molecule that increases SMN function and/or the recombinant SMN1 gene initiates. In some aspects, the dosing schedule of a small molecule that increases SMN function gene is maintained or changed after administration of the recombinant SMN1 gene and/or the SMN2 ASO initiates.

In some aspects, the present disclosure relates to a method of treating spinal muscular atrophy (SMA) in a subject having SMA, comprising administering an effective amount of a recombinant SMN1 gene (e.g., in a rAAV) and/or an ASO that increases full-length SMN2 mRNA (e.g., nusinersen) or separate compositions to a subject that was previously treated with a small molecule that increases SMN function (e.g., Risdiplam or Branaplam) that increases SMN function. In some aspects, the small molecule treatment is discontinued and the ASO and/or recombinant SMN1 gene can be provided as a replacement therapy. In some aspects, the small molecule treatment is continued and the ASO and/or recombinant SMN1 gene can be provided as an additional therapy (e.g., as an adjunct therapy).

Other aspects of the present disclosure relates to separate compositions comprising a small molecule that increases SMN function (e.g., Risdiplam or Branaplam), an rAAV encoding SMN1, or an ASO that is capable of increasing full-length SMN2 mRNA (e.g., nusinersen). In some aspects, the rAAV comprises AAV9 capsid proteins. In some aspects, the ASO is nusinersen. In some aspects, the small molecule is Risdiplam or Branaplam. In some aspects, the composition or separate compositions is a pharmaceutical composition and comprises a pharmaceutically acceptable carrier.

Other aspects and advantages of the invention will be readily apparent from the following detailed description of the invention.

### BRIEF DESCRIPTION OF FIGURES

The following drawings form part of the present specification and are included to further demonstrate certain aspects of the present application, which can be better understood by reference to one or more of these drawings in combination with the detailed description of specific aspects presented herein.

FIG. 1 illustrates increased levels of SMN activity in a greater number of motor neurons in a subject receiving combined treatment with a recombinant nucleic acid that encodes SMN1 and an antisense oligonucleotide (e.g., nusinersen) that increases full-length SMN2 mRNA (e.g., promotes exon 7 inclusion in SMN2 mRNA);

FIG. 2 is a schematic representation of a non-limiting example of a nucleic acid that encodes SMN1;

FIG. 3 illustrates the chemical structure of nusinersen, a non-limiting example of an antisense oligonucleotide that increases full-length SMN2 mRNA (e.g., promotes exon 7 inclusion in SMN2 mRNA);

FIGS. 4A-4B show the distribution of rAAV following different modes of administration in non-human primates. FIG. 4A shows rAAV distribution in spinal cord cervical, spinal cord thoracic and spinal cord lumbar following lumbar puncture (LP) or intra-Cisterna Magna (ICM) injection of the rAAV encoding SMN1. FIG. 4B shows rAAV distribution in spinal cord cervical, spinal cord thoracic and spinal cord lumbar following lumbar puncture (LP), intra-Cisterna Magna (ICM) injection or intravenous (IV) injection of 25 rAAV encoding SMN1.

FIGS. 5A-5E illustrate the physical and biological compatibility of a recombinant nucleic acid that encodes SMN1 and an antisense oligonucleotide that increases full-length SMN2 mRNA (e.g., promotes exon 7 inclusion in SMN2 mRNA); FIG. 5A shows the SEC-HPLC analysis of a rAAV encoding SMN1. FIG. 5B shows the SEC-HPLC analysis of an ASO that increases full-length SMN2. FIG. 5C shows the SEC-HPLC analysis of a rAAV encoding SMN1 and an ASO that increases full-length SMN2. FIG. 5D provides data for SMN1 rAAV infectivity in cells in vitro upon delivery of either the SMN1 rAAV vector alone or with the SMN2 ASO. The results show that SMN1 rAAV infectivity is not significantly affected by the presence of the SMN2 ASO in a co-formulation. FIG. 5E shows intracellular SMN protein expression level and GEM formation in cells following treatment with SMN1 rAAV, SMN2 ASO, or both.

FIGS. 6A-6B show that the administration of either an SMN1 gene (e.g., in an rAAV vector) or a SMN2 ASO (e.g., nusinersen, for example in a single dose) partially rescues motor function at postnatal day (PND) 8** with full rescue at PND 16, post dosing. They also show that body weight lags behind the WT control. FIG. 6A is a set of graphs showing the righting reflex (RR) of 4 separate groups after 8 and 16 days of ASO (nusinersen). FIG. 6B is a set of graphs showing the body weight of 4 separate groups after 8 and 16 days of ASO (nusinersen). The partial rescue of RR (PND 7-16) and body weight provides a window for an additional benefit of combination therapy in this pre-clinical model;

FIGS. 7A-7C show the results of a first study with body weight and RR as the primary end points for treatment with SMN1 gene therapy (in an rAAV vector) and SMN2 ASO (nusinersen). FIG. 7A is a graph showing the body weight change over time (in days). FIG. 7B is a graph showing the RR change over time (in days). FIG. 7C is a chart outlining conditions for the three testing groups;

FIGS. 8A-8C show the results of a second study with body weight and RR as the primary end points for treatment with SMN1 gene therapy (in an rAAV vector) and SMN2 ASO (nusinersen). FIG. 8A is a chart outlining conditions for the three testing groups. FIG. 8B is a graph showing the body weight change over time (in days). FIG. 8C is a graph showing the RR change over time (in days);

FIGS. 9A-9B show the comparison of % change in body weight from PND 7-PND 13. FIG. 9A shows the % change in body weight at a dose of gene therapy (rAAV): 1×10¹⁰ GC/ASO (nusinersen): 1 μg. FIG. 9B shows the % change in body weight a dose of gene therapy (rAAV): 3×10¹⁰ GC/ASO (nusinersen): 3 μg;

FIGS. 10A-10B show the comparison of % change in RR from PND 7-PND 13. FIG. 10A shows the % change in RR at a dose of gene therapy (rAAV): 1×10¹⁰ GC/ASO (nusinersen): 1 μg. FIG. 10B shows the % change in RR at a dose of gene therapy (rAAV): 3×10¹⁰ GC/ASO (nusinersen): 3 μg; and,

FIG. 11 illustrates a model showing complementarity in neuronal and non-neuronal cells using combination therapy for treating SMA. For example, Therapy 1 could be an ASO (e.g., SMN2 ASO), a small molecule that increases SMN function, or a combination therapy of an ASO and a small molecule that increases SMN function (e.g., administered concurrently or sequentially). Therapy 2 could be a SMN1 gene therapy, a small molecule that increases SMN function, or a combination therapy of a SMN1 gene therapy and a small molecule that increases SMN function (e.g., administered concurrently or sequentially). For examples, in some aspects, Therapy 1 is an ASO (e.g., SMN2 ASO), and Therapy 2 is a small molecule that increases SMN function. Therapy 1&2 could be any other therapy or combination therapy that includes the therapy not used in Therapy 1 or Therapy 2.

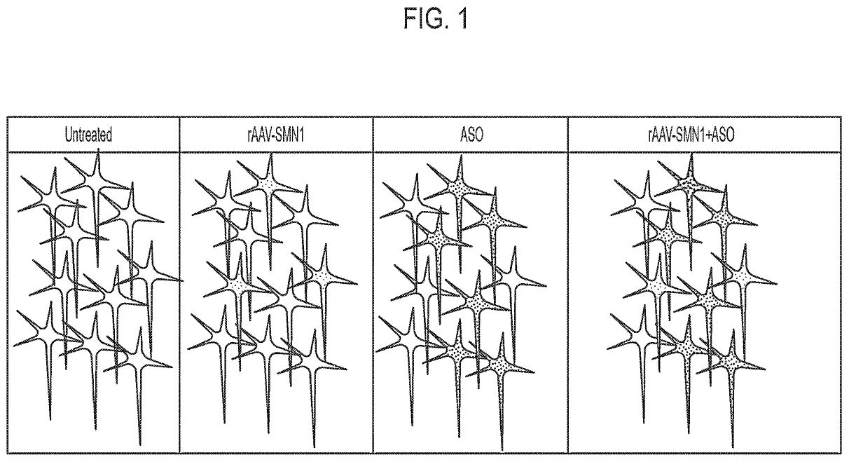

### DETAILED DESCRIPTION

In some aspects, the present application relates to compositions and methods for treating spinal muscular atrophy (SMA) in a subject, for example in a human subject having SMA.

In some aspects, the present application relates to a method of treating spinal muscular atrophy (SMA) in a subject having SMA comprising administering to the subject: a) a small molecule that increases SMN function, and b) a recombinant nucleic acid that encodes the survival of motor neuron 1 (SMN1) protein.

In some aspects, the present application relates to a method of treating spinal muscular atrophy (SMA) in a subject having SMA comprising administering to the subject: a) a small molecule that increases SMN function, and b) an antisense oligonucleotide (ASO) that increases full-length survival of motor neuron 2 (SMN2) mRNA.

In some aspects, the present application relates to a method of treating spinal muscular atrophy (SMA) in a subject having SMA comprising administering to the subject: a) a small molecule that increases SMN function, b) a recombinant nucleic acid that encodes the survival of motor neuron 1 (SMN1) protein, and c) an antisense oligonucleotide (ASO) that increases full-length survival of motor neuron 2 (SMN2) mRNA.

The present application relates to compositions and methods for treating spinal muscular atrophy (SMA) in a subject, for example in a human subject having SMA using a combination therapy.

In some aspects, a combination therapy comprises administering, to a subject having SMA (e.g., concurrently or sequentially), a small molecule that increases SMN function in a subject (e.g., Risdiplam or Branaplam) and a) a recombinant nucleic acid that expresses the SMN1 gene (e.g., in a viral vector such as an rAAV encoding SMN1) and/or b) an antisense oligonucleotide (ASO) that increases full-length SMN2 mRNA (e.g., an ASO that promotes the inclusion of exon 7 in SMN2 mRNA such as nusinersen). A “combination therapy”, a “combined treatment”, a “combined therapy”, or a “combined treatment”, as used herein, refers to a method for treating Spinal Muscular Atrophy (SMA) by administering a subject one or more of the therapies described herein (e.g., a recombinant SMN1 gene, a SMN2 ASO, a small molecule that increases SMN function, or a pharmaceutical composition of any of the foregoing).

In some aspects, administration of a small molecule capable of increasing SMN function (e.g., Risdiplam or Branaplam) and a recombinant nucleic acid that expresses SMN1 (e.g., in a rAAV) and/or a SMN2 ASO (e.g., nusinersen) can provide enhanced intracellular SMN protein levels in some motor neuron and also increase the number of motor neurons in which intracellular survival-of-motor-neuron (SMN) protein levels are elevated relative to treatment with any of the recombinant nucleic acid, SMN2 ASO (e.g., nusinersen), or small molecule that increases SMN function (e.g., Risdiplam or Branaplam) alone.

Methods and compositions for administration of a small molecule capable of increasing SMN function (e.g., Risdiplam or Branaplam), and/or a recombinant nucleic acid that expressed SMN1 (e.g., in a rAAV), and/or an ASO that increases full-length SMN2 mRNA (e.g., an ASO that promotes the inclusion of exon 7 in SMN2 mRNA such as nusinersen) can be useful to provide therapeutically effective levels of SMN protein in a subject having SMA, and also to treat subjects having different levels of disease severity.

Spinal muscular atrophy or proximal spinal muscular atrophy (SMA) is a genetic, neurodegenerative disorder characterized by the loss of spinal motor neurons. SMA is an autosomal recessive disease of early onset and is currently a leading cause of death among infants. The severity of SMA varies among patients and has thus been classified into different types depending on the age of onset and motor development milestones. SMA 0 designation has been proposed to reflect prenatal onset and severe joint contractures, facial diplegia, and respiratory failure. Three types of post-natal form of SMA have been designated. Type I SMA (also called Werdnig-Hoffmann disease) is the most severe form with onset at birth or within 6 months and typically results in death within 2 years. Children with type I SMA are unable to sit or walk and have serious respiratory dysfunction. Type II SMA is the intermediate form with onset within the first 2 years. Children with Type II SMA are able to sit, but cannot stand or walk. Type III (also called Kugelberg-Welander disease) begins after 18 months to 2 years of age (Lefebvre et al., Hum. Mol. Genet., 1998, 7, 1531-1536) and usually has a chronic evolution. Children with Type III SMA can stand and walk unaided at least in infancy. Adult form (type IV) is the mildest form of SMA, with onset after 30 years of age, and few cases have been reported. Type III and type IV SMA are also known as later-onset SMA.

The molecular basis of SMA results from the loss of both copies of survival motor neuron gene 1 (SMN1), which may also be known as SMN Telomeric, a protein that is part of a multi-protein complex thought to be involved in snRNP biogenesis and recycling. A nearly identical gene, SMN2, which may also be known as SMN Centromeric, exists in a duplicated region on chromosome 5q13 and modulates disease severity. Expression of the normal SMN1 gene results solely in expression of survival motor neuron (SMN) protein. Although SMN1 and SMN2 have the potential to code for the same protein, SMN2 contains a translationally silent mutation at position +6 of exon 7, which results in inefficient inclusion of exon 7 in SMN2 transcripts. Thus, the predominant form of SMN2 is a truncated version, lacking exon 7, which is unstable and inactive (Cartegni and Krainer, Nat. Genet., 2002, 30, 377-384). Expression of the SMN2 gene results in approximately 10-20% of the SMN protein and 80-90% of the unstable/non-functional SMN delta 7 protein. SMN protein plays a well-established role in assembly of the spliceosome and may also mediate mRNA trafficking in the axon and nerve terminus of neurons.

Although SMA is caused by the homozygous loss of both functional copies of the SMN1 gene, the SMN2 gene has the potential to code for the same protein as SMN1 and thus overcome the genetic defect of SMA patients. SMN2 contains a translationally silent mutation (C→T) at position +6 of exon 7, which results in inefficient inclusion of exon 7 in SMN2 transcripts. Therefore, the predominant form of SMN2, one which lacks exon 7, is unstable and inactive. The full-size protein made from the SMN2 gene is identical to the protein made from a similar gene called SMN1. However, only 10 to 15 percent of all functional SMN protein is produced from the SMN2 gene (the rest is produced from the SMN1 gene). Typically, people have two copies of the SMN1 gene and one to two copies of the SMN2 gene in each cell. However, the number of copies of the SMN2 gene varies, with some people having up to eight copies. The more SMN2 gene copies a person has, the more SMN protein they produce. Extra copies of the SMN2 gene can modify the severity of SMA. Since all individuals with spinal muscular atrophy have mutations in both copies of the SMN1 gene, which leads to little or no SMN protein is produced from SMN1, the SMN2 gene can help replace some of the missing SMN protein. In people with spinal muscular atrophy, having multiple copies of the SMN2 gene is usually associated with less severe features of the condition that develop later in life. Affected individuals with one or two functional copies of the SMN2 gene generally have severe muscle weakness that begins at birth or in infancy. Affected individuals with four or more copies of the SMN2 gene typically have mild muscle weakness that may not become noticeable until adulthood. In some aspects, different doses and/or designs of one or more treatments described herein may be administered to different subjects having different numbers of SMN2 genes.

In some aspects, intracellular SMN protein levels can be increased by contacting motor neurons with a small molecule capable of increasing SMN function (e.g., Risdiplam or Branaplam), and a) a recombinant nucleic acid that encodes a recombinant SMN1 gene to promote intracellular expression of a recombinant SMN protein, and/or b) an ASO that modulates intracellular SMN2 splicing such that the percentage of cellular SMN2 transcripts containing exon 7 is increased, thereby resulting in increased expression of full length SMN protein from cellular SMN2 transcripts. In some aspects, a combination therapy comprises administering a small molecule capable of increasing SMN function, a recombinant nucleic acid that encodes an SMN1 gene (also referred to herein as a recombinant SMN1 gene), and a SMN2 ASO that increases full-length SMN2 mRNA (e.g., an ASO that increases the intracellular level of full-length SMN2 mRNA, for example by promoting the inclusion of exon 7 in SMN2 mRNA). In some aspects, the SMN2 mRNA is nusinersen. In some aspects, increasing intracellular levels of full-length SMN2 mRNA is useful to target multiple aspects of SMA and can be useful for treating a range of subjects having different disease severities including patients having different types of SMA, including patients having different genomic copy numbers of the SMN2 gene. In some aspects, the small molecule that increases SMN function and the recombinant SMN1 gene are administered concurrently. In some aspects, the small molecule that increases SMN function and SMN2 ASO are administered concurrently. In some aspects, the small molecule that increases SMN function, the recombinant SMN1 gene and the SMN2 ASO are administered concurrently. In some aspects, the small molecule that increases SMN function and the recombinant SMN1 gene are administered sequentially. In some aspects, the small molecule that increases SMN function and SMN2 ASO are administered sequentially. In some aspects, the small molecule that increases SMN function, the recombinant SMN1 gene and the SMN2 ASO are administered sequentially.

In some aspects, the small molecule that increases SMN function, the recombinant SMN1 gene, or the SMN2 ASO are formulated separately. In some aspects, the route of administration for each a molecule can be different and is dictated by the type of molecule being administered to a subject (e.g., known methods suitable for administering a recombinant gene, a small molecule or an antisense oligonucleotide).

In some aspects, a recombinant SMN1 gene (e.g., in a rAAV) is formulated as a pharmaceutical composition suitable for delivering the recombinant gene to a subject. The administration of the recombinant SMN1 gene can be via any known route suitable for administering a recombinant SMN1 gene. In some aspects, the pharmaceutical composition comprising the recombinant SMN1 gene is suitable for rAAV-based delivery (e.g., an injectable solution). In some aspects, the administration of a recombinant SMN1 gene (e.g., in an rAAV) for treating SMA is by injection (e.g., via intravenous injection, direct injection to the CNS, or any other suitable route).

In some aspects, a small molecule that increases SMN function (e.g., Risdiplam or Branaplam) is formulated as a pharmaceutical composition suitable for delivering a small molecule drug to a subject (e.g., in the form of one or more tablets, pills, capsules, powders, granules, or solutions, etc.). The administration of the small molecule that increases SMN function can be via any known route suitable for administering a small molecule drug (e.g., oral administration). In some aspects, the small molecule that increases SMN function is given to the subject by oral administration.

In some aspects, a SMN2 ASO (e.g., nusinersen) is formulated as a pharmaceutical composition suitable for delivering an oligonucleotide (e.g., as an injectable solution). The administration of the SMN2 ASO (e.g., nusinersen) can be via any known route suitable for administering an ASO. In some aspects, the SMN2 ASO for treating SMA is administered to the subject by intracerebroventricular (ICV) injection, intravenous (IV) injection, or intrathecal (IT) injection (e.g., via lumbar puncture (LP), and/or intracisternal magna (ICM) delivery). In some aspects, the SMN2 ASO for treating SMA is administered to the subject by intrathecal (IT) injection.

In some aspects, any of the pharmaceutical compositions described herein further comprises a pharmaceutically acceptable carrier (e.g., excipient). A pharmaceutical acceptable carrier, as used herein, refers to a carrier that is compatible with the active ingredient and/or gene therapy agent (e.g., the rAAV) of the composition (and preferably, capable of stabilizing the active ingredient) and not deleterious to the subject to be administered. Pharmaceutically acceptable carriers can be any suitable pharmaceutically acceptable carrier known in the art including, but not limited to, excipients, buffers, one or more suitable salts, surface-active agents, antioxidants, etc.

Pharmaceutical compositions to be used in the present methods can comprise pharmaceutically acceptable carriers, excipients, or stabilizers in the form of lyophilized formulations or aqueous solutions. (Remington: The Science and Practice of Pharmacy 20th Ed. (2000) Lippincott Williams and Wilkins, Ed. K. E. Hoover).

Pharmaceutical compositions to be used for in vivo administration may be sterile. This can be accomplished by any means known in the art including, but not limited to, filtration through sterile filtration membranes.

Pharmaceutical compositions described herein can be in suitable unit dosage forms known in the art such as, but not limited to tablets, pills, capsules, powders, granules, solutions or suspensions, or suppositories.

In some aspects, a combined treatment comprises administering a first composition comprising the small molecule that increases SMN function and a separate second composition comprising the recombinant SMN1 gene (e.g., concurrently or sequentially. In some aspects, a combined treatment comprises administering a first composition comprising the small molecule that increases SMN function and a separate second composition comprising the SMN2 ASO (e.g., concurrently or sequentially). In some aspects, a combined treatment comprises administering a first composition comprising the small molecule that increases SMN function, a separate second composition comprising the recombinant SMN1 gene, and a separate third composition comprising the SMN2 ASO (e.g., concurrently or sequentially). In some aspects, the first and second compositions are administered concurrently, as defined herein). In some aspects, the first, second, and third compositions are administered concurrently, as defined herein. In some aspects, the first and second compositions are administered to a subject sequentially, as defined herein. In some aspects, the first, second, and third compositions are administered to a subject sequentially, as defined herein.

Concurrent administration, as used herein, refers to administration of two or more of therapies described herein (e.g., a recombinant SMN1 gene, a SMN2 ASO or a small molecule that increases SMN function) for treating SMA to a subject simultaneously or at different times during the same medical visit. For example during the same visit to a hospital, clinic, or other medical center, the subject is administered two or more of the therapies described herein, but the administrations can be spaced apart as dictated by the individual therapy.

Sequential administration, as used herein, refers to administration of two or more of the therapies described herein (e.g., a recombinant SMN1 gene, a SMN2 ASO or a small molecule that increases SMN function) for treating SMA under different dosing schedules. For example the therapies may be administered on different days, weeks, months, or years during different medical visits. The therapies described herein can be administered to the subject in any order (e.g., determined in a treatment plan by a physician). In some aspects, sequential administration include administration of each of the recombinant SMN1 gene, the SMN2 ASO and/or the small molecule that increases SMN function described herein at different frequencies or dosing schedules.

Accordingly, in some aspects, a first and second compositions, as described herein, are administered to the subject separately at different times (e.g., at different times of a day, on different days in the same week or month, or on different weeks, months, or years). In some aspects, a first, second, and third composition, as described herein, are administered to the subject separately at different times (e.g., at different times of a day, on different days in the same week, or on different weeks). In some aspects, a first and second compositions, as described herein are administered at different frequencies. In some aspects, a first, second and third compositions, as described herein, are administered at different frequencies. In some aspects, a composition comprising the recombinant SMN1 gene (e.g., in a rAAV) is administered less frequently than a composition comprising the small molecule that increases SMN function (e.g., Risdiplam or Branaplam). In some aspects, a composition comprising the recombinant SMN1 gene (e.g., in a rAAV) is administered less frequently than a composition comprising a SMN2 ASO (e.g., nusinersen) or a composition comprising a small molecule that increases SMN function (e.g., Risdiplam or Branaplam).

In some aspects, a recombinant SMN1 gene is administered to a subject before the subject is treated with a small molecule that increases SMN function or a SMN2 ASO. However, in other aspects a subject is already being treated with a small molecule that increases SMN function and/or a SMN2 ASO before being administered a recombinant SMN1 gene. In some aspects, a recombinant SMN1 gene is administered to a subject already receiving a small molecule that increases SMN function and/or a SMN2 ASO.

In some aspects, a small molecule that increases SMN function is administered to a subject before the subject is treated with a recombinant SMN1 gene and/or a SMN2 ASO. However, in other aspects a subject is treated with a recombinant SMN1 gene and/or a SMN2 ASO before being administered a small molecule that increases SMN function. In some aspects, a small molecule that increases SMN function is administered to a subject already receiving a recombinant SMN1 gene and/or a SMN2 ASO treatment. In some aspects, a SMN2 ASO is administered to a subject before the subject is treated with a recombinant SMN1 gene and/or a small molecule that increases SMN function. However, in other aspects a subject is treated with a recombinant SMN1 gene and/or a small molecule that increases SMN function before being administered a SMN2 ASO. In some aspects, a SMN2 ASO is administered to a subject already receiving a recombinant SMN1 gene and/or a small molecule that increases SMN function.

In some aspects, one, two or more subsequent doses of recombinant SMN1 gene (e.g., in a rAAV) or SMN2 ASO alone, or recombinant SMN1 gene (e.g., in a rAAV) and SMN2 ASO (e.g., nusinersen) are administered following an initial administration of a small molecule that increases SMN function (e.g., Risdiplam or Branaplam). In some aspects, one, two or more subsequent doses of small molecule that increases SMN function (e.g., Risdiplam or Branaplam) or SMN2 ASO (e.g., nusinersen) alone, or small molecule that increases SMN function (e.g., Risdiplam or Branaplam) and SMN2 ASO (e.g., nusinersen) are administered following an initial administration of recombinant SMN1 gene (e.g., in a rAAV). In some aspects, one, two or more subsequent doses of small molecule that increases SMN function (e.g., Risdiplam or Branaplam) or recombinant SMN1 gene, or small molecule that increases SMN function (e.g., Risdiplam or Branaplam) and recombinant SMN1 gene (e.g., in a rAAV) are administered following an initial administration of SMN2 ASO (e.g., nusinersen). In some aspects, one, two or more subsequent doses of small molecule that increases SMN function (e.g., Risdiplam or Branaplam) are administered following an initial administration of recombinant SMN1 gene (e.g., in a rAAV) and SMN2 ASO (e.g., nusinersen). In some aspects, recombinant SMN1 gene (e.g., in a rAAV) and SMN2 ASO (e.g., nusinersen) are administered following an initial administration of small molecule that increases SMN function (e.g., Risdiplam or Branaplam) alone.

A variety of assays exist for measuring SMN expression and activity levels in vitro. See, e.g., Tanguy et al, 2015, cited above. The methods described herein can also be combined with any other therapy for treatment of SMA or the symptoms thereof. See, also, Wang et al, Consensus Statement for Standard of Care in Spinal Muscular Atrophy, which provides a discussion of the present standard of care for SMA and http://www.ncbi.nlm.nih.gov/books/NBK1352/(Prior T W, Leach M E, Finanger E. Spinal Muscular Atrophy. 2000 Feb. 24. GeneReviews). For example, when nutrition is a concern in SMA, placement of a gastrostomy tube is appropriate. As respiratory function deteriorates, tracheotomy or noninvasive respiratory support is offered. Sleep-disordered breathing can be treated with nighttime use of continuous positive airway pressure. Surgery for scoliosis in individuals with SMA II and SMA III can be carried out safely if the forced vital capacity is greater than 30%-40%. A power chair and other equipment may improve quality of life. See also, U.S. Pat. No. 8,211,631, which is incorporated herein by reference.

### Small Molecule Capable of Increasing SMN Function

In some aspects, a pharmaceutical composition comprises a small molecule that increases SMN function (e.g., Risdiplam or Branaplam) and is used in combination (e.g., in concurrent or sequential treatments) with (i) a pharmaceutical composition(s) that comprise a recombinant SMN1 gene (e.g., in an rAAV) and/or (ii) a pharmaceutical composition(s) that comprises a SMN2 ASO to treat SMA in a subject.

In some aspects, a small molecule drug that increases SMN function can modulate splicing, stabilize, and/or increase transcription or translation of an SMN gene (e.g., SMN1 or SMN2). In some aspects, a small molecule drug that increases SMN function can improve the activity (e.g., potency and/or efficacy) of other active agents in the composition (e.g., a recombinant SMN1 gene (e.g., in an rAAV), a SMN2 ASO when administered to a subject in need thereof.

In some aspects, the small molecule drug that increases SMN function is a splice modulator. In some aspects, the splice modulator is a SMN2 splice modulator. In some aspects, the splice modulator is a 7-Disubstituted-phenyl tetracycline. Non-limiting examples of 7-Disubstituted-phenyl tetracycline SMN2 splice modulators are described in WO 2013/181391, the contents of which are incorporated by reference. In some aspects, the splice modulator is a substituted isoindolinone. Non-limiting examples of substituted isoindolinone SMN2 splice modulators are described in US 2009/0031435, the contents of which are incorporated by reference. In some aspects, the splice modulator is a substituted carbazole derivative. Non-limiting examples of substituted carbazole derivatives that act as SMN2 splice modulators are described in WO 2005/023255, the contents of which are incorporated by reference. In some aspects, the SMN2 splice modulators are substituted 1,4-diazepanes. Non-limiting examples of substituted 1,4-diazepanes that act as SMN2 splice modulators are described in WO 2019/028440, the contents of which are incorporated by reference. In some aspects, the SMN2 splice modulators are substituted pyridazines. Non-limiting examples of substituted pyridazines that act as SMN2 splice modulators are described in WO 2015/017589, WO 2014/028459, U.S. Pat. Nos. 10,195,196, 9,545,404, 8,729,263 and WO 2015/173181 the contents of each of which are incorporated by reference.

In some aspects, the substituted pyridazine is a compound of Formula (I′):

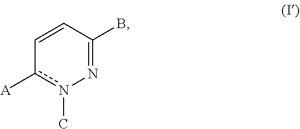

or a pharmaceutically acceptable salt thereof, wherein:

A is 2-hydroxy-phenyl which is substituted with 0, 1, 2, or 3 substituents independently selected from C₁-C₄alkyl, wherein 2 C₁-C₄alkyl groups can combine with the atoms to which they are bound to form a 5 to 6 membered ring and is substituted with 0 or 1 substituents selected from oxo, oxime and hydroxy, haloC₁-C₄alkyl, dihaloC₁-C₄alkyl, trihaloC₁-C₄alkyl, C₁-C₄alkoxy, C₁-C₄alkoxy-, C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH, C₁-C₄alkyl, -heteroaryl, —NHC(O)—, C₁-C₄alkyl-, heteroaryl, C₁-C₄alkyl-C(O)NH—, heteroaryl, C₁-C₄alkyl NHC(O)-heteroaryl, 3-7 membered cycloalkyl, 5-7 membered cycloalkenyl or 5, 6, or 9 membered heterocycle containing 1 or 2 heteroatoms, independently, selected from S, O and N, wherein heteroaryl has 5, 6, or 9 ring atoms, 1, 2, or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7member heterocycleC₁-C₄alkyl, aminoC₁-C₄alkyl and mono- and di-C₁-C₄alkylaminoC1-C₄alkyl; or

A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₅alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, alkylene 4 to 7 member heterocycle, 4 to 7 member heterocycle and mono- and di-C₁-C₄alkylamino; or

A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O, and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC1-C₄alkyl; or

A is bicyclic heteroaryl having 9 to 10 ring atoms and 1, 2, or 3 ring heteroatoms independently selected from N, O, or S, which bicyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino; or

A is tricyclic heteroaryl having 12 or 13 ring atoms and 1, 2, or 3 ring heteroatoms independently selected from N, O, or S, which tricyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy, C₁-C₄alkoxy substituted with hydroxy, C₁-C₄alkoxy, amino, mono- and di-C₁-C₄alkylamino and heteroaryl, wherein said heteroaryl has 5, 6, or 9 ring atoms, 1, 2, or 3 ring heteroatoms selected from N, O, and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC1-C₄alkyl;

B is a group of the formula:

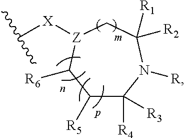

wherein:

m, n and p are independently selected from 0 or 1;

R, R₁, R₂, R₃, and R₄ are independently selected from the group consisting of hydrogen, C₁-C₄alkyl, which alkyl is optionally substituted with hydroxy, amino, or mono- and di-C₁-C₄alkylamino;

R₅ and R₆ are independently selected from hydrogen and fluorine; or

R and R₃, taken in combination form a fused 5 or 6 member heterocyclic ring having 0 or 1 additional ring heteroatoms selected from N, O, or S;

R₁ and R₃, taken in combination form a C₁-C₃alkylene group;

R₁ and R₅, taken in combination form a C₁-C₃alkylene group;

R₃ and R₄, taken in combination with the carbon atom to which they attach, form a spirocyclicC₃-C₆cycloalkyl;

X is CR_{A}R_{B}, O, NR₇, or a bond;

R₇ is hydrogen or C₁-C₄alkyl;

R_{A} and R_{B} are independently selected from hydrogen and C₁-C₄alkyl, or R_{A} and R_{B}, taken in combination, form a divalent C₂-C₅alkylene group;

Z is CR₈ or N; when Z is N, X is a bond;

R₈ is hydrogen or taken in combination with R₆ form a double bond; or

B is a group of the formula:

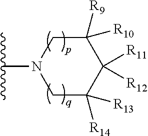

wherein:

p and q are independently selected from the group consisting of 0, 1, and 2;

R₉ and R₁₃ are independently selected from hydrogen and C₁-C₄alkyl;

R₁₀ and R₁₄ are independently selected from hydrogen, amino, mono- and di-C₁-C₄alkylamino, and C₁-C₄alkyl, which alkyl is optionally substituted with hydroxy, amino or mono- and di-C₁-C₄alkylamino;

R₁₁ is hydrogen, C₁-C₄alkyl, amino, or mono- and di-C₁-C₄alkylamino;

R₁₂ is hydrogen or C₁-C₄alkyl; or

R₉ and R₁₀, taken in combination form a saturated azacycle having 4 to 7 ring atoms, which is optionally substituted with 1-3 C₁-C₄alkyl groups; or

R₁₁ and R₁₂, taken in combination form a saturated azacycle having 4 to 7 ring atoms which is optionally substituted with 1-3 C₁-C₄alkyl groups; and C is H or absent, as valency permits.

In some aspects, the substituted pyridazine is a compound of Formula (I):

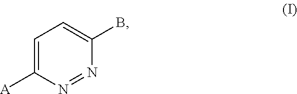

or a pharmaceutically acceptable salt thereof, wherein:

A is 2-hydroxy-phenyl which is substituted with 0, 1, 2, or 3 substituents independently selected from C₁-C₄alkyl, wherein 2 C₁-C₄alkyl groups can combine with the atoms to which they are bound to form a 5 to 6 membered ring and is substituted with 0 or 1 substituents selected from oxo, oxime and hydroxy, haloC₁-C₄alkyl, dihaloC₁-C₄alkyl, trihaloC₁-C₄alkyl, C₁-C₄alkoxy, C₁-C₄alkoxy-, C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH, C₁-C₄alkyl, -heteroaryl, —NHC(O)—, C₁-C₄alkyl-, heteroaryl, C₁-C₄alkyl-C(O)NH—, heteroaryl, C₁-C₄alkyl NHC(O)— heteroaryl, 3-7 membered cycloalkyl, 5-7 membered cycloalkenyl or 5, 6 or 9 membered heterocycle containing 1 or 2 heteroatoms, independently, selected from S, O and N, wherein heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7member heterocycleC₁-C₄alkyl, aminoC₁-C₄alkyl and mono- and di-C₁-C₄alkylaminoC1-C₄alkyl; or

A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₅alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, alkylene 4 to 7 member heterocycle, 4 to 7 member heterocycle and mono- and di-C₁-C₄alkylamino; or A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl; or

A is bicyclic heteroaryl having 9 to 10 ring atoms and 1, 2, or 3 ring heteroatoms independently selected from N, O or S, which bicyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino; or A is tricyclic heteroaryl having 12 or 13 ring atoms and 1, 2, or 3 ring heteroatoms independently selected from N, O, or S, which tricyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy, C₁-C₄alkoxy substituted with hydroxy, C₁-C₄alkoxy, amino, mono- and di-C₁-C₄alkylamino and heteroaryl, wherein said heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7member heterocycleC₁-C₄alkyl, aminoC₁-C₄alkyl and mono- and di-C₁-C₄alkylaminoC₁-C₄alkyl;

B is a group of the formula:

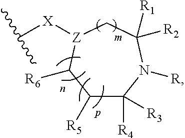

wherein:

m, n and p are independently selected from 0 or 1;

R, R₁, R₂, R₃, and R₄ are independently selected from the group consisting of hydrogen, C₁-C₄alkyl, which alkyl is optionally substituted with hydroxy, amino or mono- and di-C₁-C₄alkylamino;

R₅ and R₆ are independently selected from hydrogen and fluorine; or

R and R₃, taken in combination form a fused 5 or 6 member heterocyclic ring having 0 or 1 additional ring heteroatoms selected from N, O or S;

R₁ and R₃, taken in combination form a C₁-C₃alkylene group;

R₁ and R₅, taken in combination form a C₁-C₃alkylene group;

R₃ and R₄, taken in combination with the carbon atom to which they attach, form a spirocyclicC₃-C₆cycloalkyl;

X is CR_{A}R_{B}, O, NR₇ or a bond;

R₇ is hydrogen, or C₁-C₄alkyl;

R_{A} and R_{B} are independently selected from hydrogen and C₁-C₄alkyl, or R_{A} and R_{B}, taken in combination, form a divalent C₂-C₅alkylene group;

Z is CR₈ or N; when Z is N, X is a bond;

R₈ is hydrogen or taken in combination with R₆ form a double bond; or

B is a group of the formula:

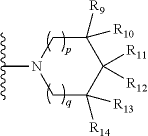

wherein:

p and q are independently selected from the group consisting of 0, 1, and 2;

R₉ and R₁₃ are independently selected from hydrogen and C₁-C₄alkyl;

R₁₀ and R₁₄ are independently selected from hydrogen, amino, mono- and di-C₁-C₄alkylamino and C₁-C₄alkyl, which alkyl is optionally substituted with hydroxy, amino or mono- and di-C₁-C₄alkylamino;

R₁₁ is hydrogen, C₁-C₄alkyl, amino or mono- and di-C₁-C₄alkylamino;

R₁₂ is hydrogen or C₁-C₄alkyl; or

R₉ and R₁₀, taken in combination form a saturated azacycle having 4 to 7 ring atoms, which is optionally substituted with 1-3 C₁-C₄alkyl groups; or R₁₁ and R₁₂, taken in combination form a saturated azacycle having 4 to 7 ring atoms which is optionally substituted with 1-3 C₁-C₄alkyl groups.

In some aspects, A is 2-hydroxy-phenyl which is substituted with 0, 1, 2, or 3 substituents independently selected from C₁-C₄alkyl, wherein 2 C₁-C₄alkyl groups can combine with the atoms to which they are bound to form a 5 to 6 membered ring and is substituted with 0 or 1 substituents selected from oxo, oxime and hydroxy, haloC₁-C₄alkyl, dihaloC₁-C₄alkyl, trihaloC₁-C₄alkyl, C₁-C₄alkoxy, C₁-C₄alkoxy-, C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH, C₁-C₄alkyl, -heteroaryl, —NHC(O)—, C₁-C₄alkyl-, heteroaryl, C₁-C₄alkyl-C(O)NH—, heteroaryl, C₁-C₄alkyl NHC(O)— heteroaryl, 3-7 membered cycloalkyl, 5-7 membered cycloalkenyl or 5, 6 or 9 membered heterocycle containing 1 or 2 heteroatoms, independently, selected from S, O and N, wherein heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7member heterocycleC₁-C₄alkyl, aminoC₁-C₄alkyl and mono- and di-C₁-C₄alkylaminoC₁-C₄alkyl. In some aspects, A is of the formula:

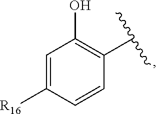

wherein R₁₆ is a 5 member heteroaryl having one ring nitrogen atom and 0 or 1 additional ring heteroatom selected from N, O or S, wherein the heteroaryl is optionally substituted with C₁-C₄alkyl. In some aspects, A is of the formula:

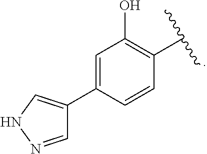 [SMILES: *c1ccc(-c2cn[nH]c2)cc1O]

In some aspects, A is of the formula:

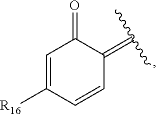

wherein R₁₆ is a 5 member heteroaryl having one ring nitrogen atom and 0 or 1 additional ring heteroatom selected from N, O or S, wherein the heteroaryl is optionally substituted with C₁-C₄alkyl. In some aspects, A is of the formula:

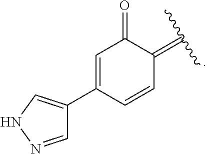 [SMILES: *=C1C=CC(c2cn[nH]c2)=CC1=O]

In some aspects, A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₅alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, alkylene 4 to 7 member heterocycle, 4 to 7 member heterocycle and mono- and di-C₁-C₄alkylamino.

In some aspects, A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl.

In some aspects, A is bicyclic heteroaryl having 9 to 10 ring atoms and 1, 2, or 3 ring heteroatoms independently selected from N, O or S, which bicyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino.

In some aspects, A is tricyclic heteroaryl having 12 or 13 ring atoms and 1, 2, or 3 ring heteroatoms independently selected from N, O, or S, which tricyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy, C₁-C₄alkoxy substituted with hydroxy, C₁-C₄alkoxy, amino, mono- and di-C₁-C₄alkylamino and heteroaryl, wherein said heteroaryl has 5, 6 or 9 ring atoms, 1, 2 or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7member heterocycleC₁-C₄alkyl, aminoC₁-C₄alkyl and mono- and di-C₁-C₄alkylaminoC₁-C₄alkyl.

In some aspects, B is a group of the formula:

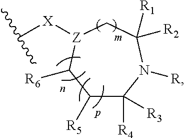

wherein:

m, n and p are independently selected from 0 or 1;

R, R₁, R₂, R₃, and R₄ are independently selected from the group consisting of hydrogen, C₁-C₄alkyl, which alkyl is optionally substituted with hydroxy, amino or mono- and di-C₁-C₄alkylamino;

R₅ and R₆ are independently selected from hydrogen and fluorine; or

R and R₃, taken in combination form a fused 5 or 6 member heterocyclic ring having 0 or 1 additional ring heteroatoms selected from N, O or S;

R₁ and R₃, taken in combination form a C₁-C₃alkylene group;

R₁ and R₅, taken in combination form a C₁-C₃alkylene group;

R₃ and R₄, taken in combination with the carbon atom to which they attach, form a spirocyclicC₃-C₆cycloalkyl;

X is CR_{A}R_{B}, O, NR₇ or a bond;

R₇ is hydrogen, or C₁-C₄alkyl;

R_{A} and R_{B} are independently selected from hydrogen and C₁-C₄alkyl, or R_{A} and R_{B}, taken in combination, form a divalent C₂-C₅alkylene group;

Z is CR₈ or N; when Z is N, X is a bond;

R₈ is hydrogen or taken in combination with R₆ form a double bond.

In some aspects, B is a group of the formula:

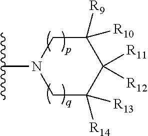

wherein:

p and q are independently selected from the group consisting of 0, 1, and 2;

R₉ and R₁₃ are independently selected from hydrogen and C₁-C₄alkyl;

R₁₀ and R₁₄ are independently selected from hydrogen, amino, mono- and di-C₁-C₄alkylamino and C₁-C₄alkyl, which alkyl is optionally substituted with hydroxy, amino or mono- and di-C₁-C₄alkylamino;

R₁₁ is hydrogen, C₁-C₄alkyl, amino or mono- and di-C₁-C₄alkylamino;

R₁₂ is hydrogen or C₁-C₄alkyl; or

R₉ and R₁₀, taken in combination form a saturated azacycle having 4 to 7 ring atoms, which is optionally substituted with 1-3 C₁-C₄alkyl groups; or

R₁₁ and R₁₂, taken in combination form a saturated azacycle having 4 to 7 ring atoms which is optionally substituted with 1-3 C₁-C₄alkyl groups.

In some aspects, B is

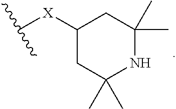

In some aspects, B

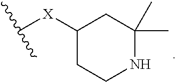

In some aspects, B is

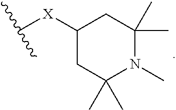

In some aspects, B is

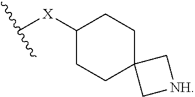

In some aspects, B is

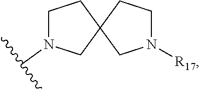

wherein R₁₇ is H or unsubstituted methyl. In some aspects, B is

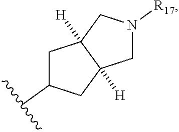

wherein R₁₇ is H or unsubstituted methyl. In some aspects, B is

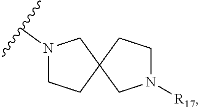

wherein R₁₇ is H or unsubstituted methyl. In some aspects, B is

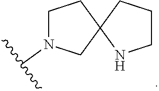 [SMILES: *N1CCC2(CCCN2)C1]

In some aspects, B is

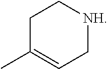

In some aspects, B is

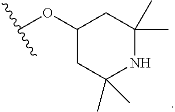 [SMILES: *OC1CC(C)(C)NC(C)(C)C1]

In some aspects, the substituted pyridazine of Formula (I′) is of Formula (II′):

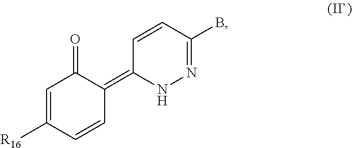

or a pharmaceutically acceptable salt thereof, wherein:

R₁₆ is a 5 member heteroaryl having one ring nitrogen atom and 0 or 1 additional ring heteroatom selected from N, O, or S, wherein the heteroaryl is optionally substituted with C₁-C₄alkyl.

In some aspects, the substituted pyridazine of Formula (I) is of Formula (II):

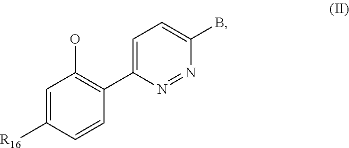

or a pharmaceutically acceptable salt thereof, wherein:

R₁₆ is a 5 member heteroaryl having one ring nitrogen atom and 0 or 1 additional ring heteroatom selected from N, O or S, wherein the heteroaryl is optionally substituted with C₁-C₄alkyl. In some aspects, R₁₆ is thiophene, furan, pyrrole, dihydropyrrole, imidazole, pyrazole, pyrazine, isothiazole, isoxazole, triazole, tetrazole, oxazole, isoxazole, thiazole, isothiazole. In some aspects, is pyrazole. In some aspects, R₁₆ is

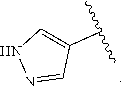 [SMILES: *c1cn[nH]c1]

In some aspects, the substituted pyridazine of Formula (I) is of the formula:

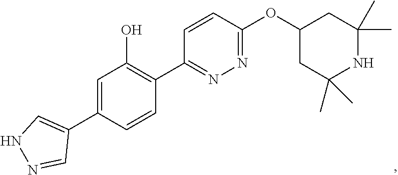 [SMILES: CC1(C)CC(Oc2ccc(-c3ccc(-c4cn[nH]c4)cc3O)nn2)CC(C)(C)N1]

or a pharmaceutically acceptable salt thereof.

In some aspects, the substituted pyridazine of Formula (I) is of the formula:

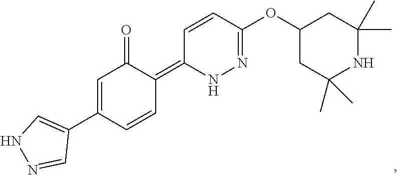 [SMILES: CC1(C)CC(OC2=NN/C(=C3\C=CC(c4cn[nH]c4)=CC3=O)C=C2)CC(C)(C)N1]

or a pharmaceutically acceptable salt thereof.

In some aspects, the substituted pyridazine is a compound of Formula (III):

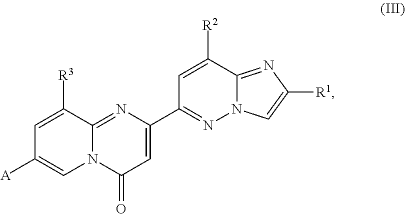

or a pharmaceutically acceptable salt thereof, wherein:

R¹ is hydrogen or C₁₋₇-alkyl;

R² is hydrogen, cyano, C₁₋₇-alkyl, C₁₋₇-haloalkyl or C₃₋₈-cycloalkyl;

R³ is hydrogen, C₁₋₇-alkyl, or C₃₋₈-cycloalkyl;

A is N-heterocycloalkyl or NR¹²R¹³, wherein N-heterocycloalkyl comprises 1 or 2 nitrogen ring atoms and is optionally substituted with 1, 2, 3 or 4 substituents selected from R¹⁴.

R¹² is heterocycloalkyl comprising 1 nitrogen ring atom, wherein heterocycloalkyl is optionally substituted with 1, 2, 3 or 4 substituents selected from R¹⁴;

R¹³ is hydrogen, C₁₋₇-alkyl or C₃₋₈-cycloalkyl;

R¹⁴ is independently selected from hydrogen, C₁₋₇-alkyl, amino, amino-C₁₋₇-alkyl, C₃₋₈-cycloalkyl and heterocycloalkyl or two R¹⁴ together form C₁₋₇-alkylene;

with the proviso that if A is N-heterocycloalkyl comprising only 1 nitrogen ring atom, then at least one R¹⁴ substituent is amino or amino-C₁₋₇-alkyl.

In some aspects, the compound of Formula (III), is of the formula:

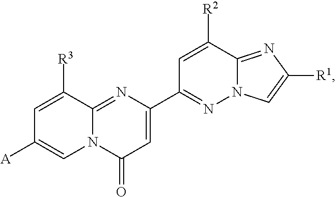

or a pharmaceutically acceptable salt thereof, wherein:

R¹ is hydrogen or C₁₋₇-alkyl;

R² is hydrogen, cyano, C₁₋₇-alkyl, C₁₋₇-haloalkyl or C₃₋₈-cycloalkyl;

R³ is hydrogen, C₁₋₇-alkyl, or C₃₋₈-cycloalkyl;

A is N-heterocycloalkyl comprising 1 or 2 nitrogen ring atoms, wherein N-heterocycloalkyl is optionally substituted with 1, 2, 3 or 4 substituents selected from R¹⁴; R¹⁴ is independently selected from hydrogen, C₁₋₇-alkyl, amino, amino-C₁₋₇-alkyl, C₃₋₈-cycloalkyl and heterocycloalkyl or two R¹⁴ together form C₁₋₇-alkylene;

with the proviso that if A is N-heterocycloalkyl comprising only 1 nitrogen ring atom, then at least one R¹⁴ substituent is amino or amino-C₁₋₇-alkyl.

In some aspects, R¹ is C₁₋₇-alkyl. In some aspects, R¹ is methyl.

In some aspects, R² is hydrogen or C₁₋₇-alkyl. In some aspects, R² is hydrogen or methyl. In some aspects, R² is hydrogen. In some aspects, R² is methyl.

In some aspects, R³ is hydrogen or C₁₋₇-alkyl. In some aspects, R³ is hydrogen or methyl. In some aspects, R³ is hydrogen. In some aspects, R³ is methyl.

In some aspects, A is N-heterocycloalkyl or NR¹²R¹³, wherein N-heterocycloalkyl comprises 1 or 2 nitrogen ring atoms and is optionally substituted with 1, 2, 3 or 4 substituents selected from R¹⁴;

R¹² is heterocycloalkyl comprising 1 nitrogen ring atom, wherein heterocycloalkyl is optionally substituted with 1, 2, 3 or 4 substituents selected from R¹⁴;

R¹³ is hydrogen, C₁₋₇-alkyl or C₃₋₈-cycloalkyl;

R¹⁴ is independently selected from hydrogen, C₁₋₇-alkyl, amino, amino-C₁₋₇-alkyl, C₃₋₈-cycloalkyl and heterocycloalkyl or two R¹⁴ together form C₁₋₇-alkylene;

with the proviso that if A is N-heterocycloalkyl comprising only 1 nitrogen ring atom, then at least one R¹⁴ substituent is amino or amino-C₁₋₇-alkyl.

In some aspects, R¹² is piperidinyl optionally substituted with 1, 2, 3 or 4 substituents selected from R¹⁴.

In some aspects, A is of the formula:

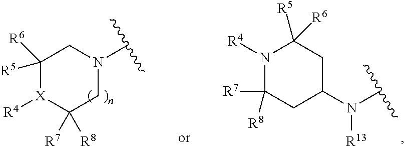

wherein:

X is N or CH;

R⁴ is hydrogen, C₁₋₇-alkyl or (CH₂)ₘ—NR⁹R¹⁰;

R⁵ is hydrogen or C₁₋₇-alkyl;

R⁶ is hydrogen or C₁₋₇-alkyl;

R⁷ is hydrogen or C₁₋₇-alkyl;

R⁸ is hydrogen or C₁₋₇-alkyl;

R⁹ and R¹⁰ are independently selected from hydrogen, C₁₋₇-alkyl and C₃₋₈-cycloalkyl;

R¹³ is hydrogen, C₁₋₇-alkyl or C₃₋₈-cycloalkyl;

n is 0, 1 or 2;

m is 0, 1, 2 or 3;

or R⁴ and R⁵ together form C₁₋₇-alkylene;

or R⁴ and R⁷ together form C₁₋₇-alkylene;

or R⁵ and R⁶ together form C₂₋₇-alkylene;

or R⁵ and R⁷ together form C₁₋₇-alkylene;

or R⁵ and R⁹ together form C₁₋₇-alkylene;

or R⁷ and R⁸ together form C₂₋₇-alkylene;

or R⁷ and R⁹ together form C₁₋₇-alkylene;

or R⁹ and R¹⁰ together form C₂₋₇-alkylene;

with the proviso that if X is CH then R⁴ is (CH₂)ₘ—NR⁹R¹⁰; and

with the proviso that if X is N and R⁴ is (CH₂)ₘ—NR⁹R¹⁰ then m is 2 or 3.

In some aspects, A is of the formula:

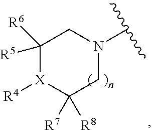

wherein:

X is N or CH;

R⁴ is hydrogen, C₁₋₇-alkyl or (CH₂)ₘ—NR⁹R¹⁰;

R⁵ is hydrogen or C₁₋₇-alkyl;

R⁶ is hydrogen or C₁₋₇-alkyl;

R⁷ is hydrogen or C₁₋₇-alkyl;

R⁸ is hydrogen or C₁₋₇-alkyl;

R⁹ and R¹⁰ are independently selected from hydrogen, C₁₋₇-alkyl and C₃₋₈-cycloalkyl;

n is 0, 1 or 2;

m is 0, 1, 2 or 3;

or R⁴ and R⁵ together form C₁₋₇-alkylene;

or R⁴ and R⁷ together form C₁₋₇-alkylene;

or R⁵ and R⁶ together form C₂₋₇-alkylene;

or R⁵ and R⁷ together form C₁₋₇-alkylene;

or R⁵ and R⁹ together form C₁₋₇-alkylene;

or R⁷ and R⁸ together form C₂₋₇-alkylene;

or R⁷ and R⁹ together form C₁₋₇-alkylene;

or R⁹ and R¹⁰ together form C₂₋₇-alkylene;

with the proviso that if X is CH then R⁴ is (CH₂)ₘ—NR⁹R¹⁰; and

with the proviso that if X is N and R⁴ is (CH₂)ₘ—NR⁹R¹⁰ then m is 2 or 3.

In some aspects, wherein X is N.

In some aspects, wherein n is 1.

In some aspects, R⁶ is hydrogen, methyl or —(CH₂)ₘ—NR⁹R¹⁰. In some aspects, R⁶ is hydrogen or methyl. In some aspects, R⁶ is hydrogen. In some aspects, R⁶ is methyl.

In some aspects, R⁷ is hydrogen or methyl.

In some aspects, m is 0.

In some aspects, R⁴ and R⁵ together form propylene. In some aspects, R⁵ and R⁶ together form ethylene. In some aspects, R⁹ and R¹⁰ together form butylene.

In some aspects, A is

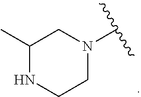 [SMILES: *N1CCNC(C)C1]

In some aspects, A is

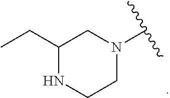 [SMILES: *N1CCNC(CC)C1]

In some aspects, A is

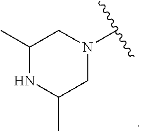 [SMILES: *N1CC(C)NC(C)C1]

In some aspects, A is

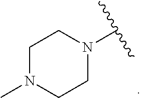 [SMILES: *N1CCN(C)CC1]

In some aspects, A is

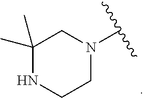 [SMILES: *N1CCNC(C)(C)C1]

In some aspects, A is

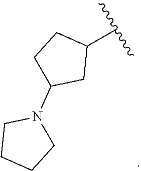 [SMILES: *C1CCC(N2CCCC2)C1]

In some aspects, A is

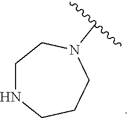 [SMILES: *N1CCCNCC1]

In some aspects, A is

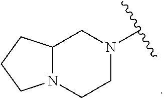 [SMILES: *N1CCN2CCCC2C1]

In some aspects, A is

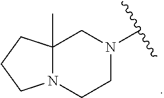 [SMILES: *N1CCN2CCCC2(C)C1]

aspects, A is

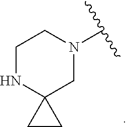 [SMILES: *N1CCNC2(CC2)C1]

In some aspects, the substituted pyridazine of Formula (III) is of the formula:

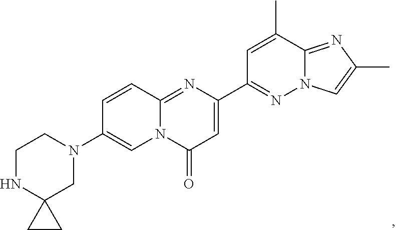 [SMILES: Cc1cn2nc(-c3cc(=O)n4cc(N5CCNC6(CC6)C5)ccc4n3)cc(C)c2n1]

or a pharmaceutically acceptable salt thereof.

In some aspects, the SMN2 splice modulator is Risdiplam. In some aspects, the SMN2 splice modulator is Branaplam.

In some aspects, the small molecule drug that that increases SMN function modulates the activity of an mRNA decapping enzyme. In some aspects, the small molecule drug inhibits the activity of an mRNA decapping enzyme. In some aspects, the small molecule drug is a DcpS inhibitor. In some aspects, the DcpS inhibitor is a C5-substituted 2,4-diaminoquinazoline (2,4-DAQ). In some aspects, the 2,4-DAQ is RG3039. In some aspects, the DcpS inhibitor is a 2,4-DAQ derivative. In some aspects, the 2,4-DAQ derivative is D156844.

In some aspects, the small molecule drug that increases SMN function is an HDAC inhibitor. In some aspects, the HDAC inhibitor is a cinamic compound and derivatives therefrom. Non-limiting examples of cinamic compounds that act as HDAC inhibitors are described in US 2010/0256401, and EP 2236503 the contents of which are incorporated by reference. In some aspects, the HDAC inhibitor is a hydroxamic acid indane derivative. Non-limiting examples of hydroxamic acid indane derivatives that act as HDAC inhibitors are described in WO 2017/218,950, the contents of which are incorporated by reference. In some aspects, the HDAC inhibitor is a 3-spiro-7-hydroxamic acid tetralins. Non-limiting examples of 3-spiro-7-hydroxamic acid tetralins that act as HDAC inhibitors are described in WO 2016/168660, the contents of which are incorporated by reference. In some aspects, the HDAC inhibitor is a 3-alkyl bicyclic [4,5,0] hydroxamic acid. Non-limiting examples of 3-alkyl bicyclic [4,5,0] hydroxamic acids that act as HDAC inhibitors are described in WO 2016/126722, the contents of which are incorporated by reference. In some aspects, the HDAC inhibitor is a fused pyrimidine hydroxamate derivative. Non-limiting examples of fused pyrimidine hydroxamate derivatives that act as HDAC inhibitors are described in US 2018/0265512, the contents of which are incorporated by reference. In some aspects, the HDAC inhibitor is a tetrahydroindole and/or tetrahydroindazole. derivatives. Non-limiting examples of tetrahydroindoles and tetrahydroindazoled that act as HDAC inhibitors are described in WO2009114470A2, the contents of which are incorporated by reference. In some aspects, the HDAC inhibitor is a benzimidazole. Non-limiting examples of benzimidazoles that act as HDAC inhibitors are described in WO 2005/028447, the contents of which are incorporated by reference. In some aspects, the HDAC inhibitor is a 2-propylpentanoic acid derivative. Non-limiting examples of 2-propylpentanoic acid that act as HDAC inhibitors are described in US 2012/0071554, the contents of which are incorporated by reference. In some aspects, the HDAC inhibitor is a pimelic acid derivative. Non-limiting examples of pimelic acid derivatives that act as HDAC inhibitors are described in WO 2010/028193, the contents of which are incorporated by reference. In some aspects, the HDAC inhibitor is a 6-aminohexanoic acids. Non-limiting examples of 6-aminohexanoic acids that act as HDAC inhibitors are described in U.S. Pat. No. 9,796,664, the contents of which are incorporated by reference. In some aspects, the HDAC inhibitor is a hydroxamic acid compound. Non-limiting examples of hydroxamic acid compounds that act as HDAC inhibitors are described in WO 2006/101456, US 2010/0261710, the contents of each of which are incorporated by reference. In some aspects, the HDAC inhibitor is a hydroxamic acid compound. Non-limiting examples of hydroxamic acid compounds that act as HDAC inhibitors are described in US 2010/0105721, US 2008/0085896, the contents of each of which are incorporated by reference. In some aspects, the HDAC inhibitor is a benzothiophene derivative. Non-limiting examples of benzothiophene derivatives that act as HDAC inhibitors are described in WO 2006/101454, the contents of which are incorporated by reference. In some aspects, the HDAC inhibitor is a heteroaryl amide derivative. Non-limiting examples of heteroaryl amide derivatives that act as HDAC inhibitors are described in WO 2019/012172, the contents of which are incorporated by reference. In some aspects, the HDAC inhibitor is a substituted bicyclic [4.6.0] hydroxamic acid. Non-limiting examples of substituted bicyclic [4.6.0] hydroxamic acids that act as HDAC inhibitors are described in US 2016/0221997, the contents of which are incorporated by reference. In some aspects, the HDAC inhibitor is an aminobenzimidazole derivative. Non-limiting examples of aminobenzimidazole derivatives that act as HDAC inhibitors are described in WO 2019/051125, the contents of which are incorporated by reference.

In some aspects, the HDAC inhibitor is an imidazo[1,2-a]pyridine derivative. Non-limiting examples of imidazo[1,2-a]pyridine derivatives that act as HDAC inhibitors are described in US 2008/0085896, the contents of which are incorporated by reference.

In some aspects, the HDAC inhibitor is a pyrimidine hydroxy compound. Non-limiting examples of pyrimidine hydroxy compounds that act as HDAC inhibitors are described in US 2017/0096403, the contents of which are incorporated by reference.

Other non-limiting examples of HDAC inhibitors small molecule drugs described in: WO 2018/165520, US 2017/0050984, US 2007/0219244, US 2017/0305900, US 2017/0224684A1, US 2008/0312175, WO 2018/129533, WO 2018/119362, WO 2018/017858, WO 2018/009531,

WO 2017/004522, US 2018/0057456, WO 2016/020369, WO 2014/143666, JP 6336562, US 2011/0300134, US 2011/0218221, U.S. Pat. No. 8,008,344, EP 2045247, JP 2009507829, CN 102271668, U.S. Pat. No. 9,855,267, US 2018/0362472, US 2017/0349573, JP 5838157, WO 2019/007836, TW 200911230, AU 2007/21678, the contents of each of which are incorporated by reference.

Exemplary HDAC inhibitors also include, but are not limited to, valproic acid, hydroxybutyrate, phenylbutyrate, phenylbutyrate derivatives, trichostatin A (TSA) and suberoylanilide hydroxamic acid (SAHA). An exemplary methylase inhibitor is 5-azacytidine.

As used herein, the term “small molecule” refers to molecules, whether naturally-occurring or artificially created (e.g., via chemical synthesis) that have a relatively low molecular weight. Typically, a small molecule is an organic compound (i.e., it contains carbon). The small molecule may contain multiple carbon-carbon bonds, stereocenters, and other functional groups (e.g., amines, hydroxyl, carbonyls, and heterocyclic rings, etc.). In certain aspects, the molecular weight of a small molecule is at most about 1,000 g/mol, at most about 900 g/mol, at most about 800 g/mol, at most about 700 g/mol, at most about 600 g/mol, at most about 500 g/mol, at most about 400 g/mol, at most about 300 g/mol, at most about 200 g/mol, or at most about 100 g/mol. In certain aspects, the molecular weight of a small molecule is at least about 100 g/mol, at least about 200 g/mol, at least about 300 g/mol, at least about 400 g/mol, at least about 500 g/mol, at least about 600 g/mol, at least about 700 g/mol, at least about 800 g/mol, or at least about 900 g/mol, or at least about 1,000 g/mol. Combinations of the above ranges (e.g., at least about 200 g/mol and at most about 500 g/mol) are also possible. In certain aspects, the small molecule is a therapeutically active agent such as a drug (e.g., a molecule approved by the U.S. Food and Drug Administration as provided in the Code of Federal Regulations (C.F.R.)). The small molecule may also be complexed with one or more metal atoms and/or metal ions. In this instance, the small molecule is also referred to as a “small organometallic molecule.” Preferred small molecules are biologically active in that they produce a biological effect in animals, preferably mammals, more preferably humans. In certain aspects, the small molecule is a drug. Preferably, though not necessarily, the drug is one that has already been deemed safe and effective for use in humans or animals by the appropriate governmental agency or regulatory body. For example, drugs approved for human use are listed by the FDA under 21 C.F.R. §§ 330.5, 331 through 361, and 440 through 460, incorporated herein by reference; drugs for veterinary use are listed by the FDA under 21 C.F.R. §§ 500 through 589, incorporated herein by reference. All listed drugs are considered acceptable for use in accordance with the present invention.

Definitions of certain functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Thomas Sorrell, Organic Chemistry, University Science Books, Sausalito, 1999; Michael B. Smith, March's Advanced Organic Chemistry, 7^{th} Edition, John Wiley & Sons, Inc., New York, 2013; Richard C. Larock, Comprehensive Organic Transformations, John Wiley & Sons, Inc., New York, 2018; and Carruthers, Some Modern Methods of Organic Synthesis, 3^{rd} Edition, Cambridge University Press, Cambridge, 1987.

Compounds described herein can comprise one or more asymmetric centers, and thus can exist in various stereoisomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, N Y, 1962); and Wilen, S. H., Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

The term “tautomers” or “tautomeric” refers to two or more interconvertible compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may be catalyzed by acid or base. Compounds described herein can comprise one or more tautomeric forms, and thus can exist as tautomers.

Exemplary tautomerizations include keto-to-enol, amide-to-imide, lactam-to-lactim, enamine-to-imine, and enamine-to-(a different enamine) tautomerizations. For example, a keto-to-enol tautomerizations can include:

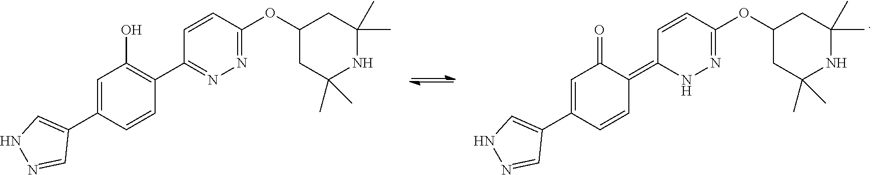

In a formula, the bond [symbol] is a single bond, the dashed line [symbol] is a single bond or absent, and the bond [symbol] or [symbol] is a single or double bond.

Unless otherwise provided, a formula includes compounds that do not include isotopically enriched atoms and also compounds that include isotopically enriched atoms. Compounds that include isotopically enriched atoms may be useful, for example, as analytical tools and/or probes in biological assays.

When a range of values (“range”) is listed, it is intended to encompass each value and sub-range within the range. A range is inclusive of the values at the two ends of the range unless otherwise provided. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂ 3, C₃₋₆, C₃₋₅, C₃₋₄, C₄₋₆, C₄₋₅, and C₅₋₆ alkyl.

The term “aliphatic” refers to alkyl, alkenyl, alkynyl, and carbocyclic groups. Likewise, the term “heteroaliphatic” refers to heteroalkyl, heteroalkenyl, heteroalkynyl, and heterocyclic groups.

The term “alkyl” refers to a radical of a straight-chain or branched saturated hydrocarbon group having from 1 to 20 carbon atoms (“C₁₋₂₀ alkyl”). In some aspects, an alkyl group has 1 to 12 carbon atoms (“C₁₋₁₂ alkyl”). In some aspects, an alkyl group has 1 to 10 carbon atoms (“C₁₋₁₀ alkyl”). In some aspects, an alkyl group has 1 to 9 carbon atoms (“C₁₋₉ alkyl”). In some aspects, an alkyl group has 1 to 8 carbon atoms (“C₁₋₈ alkyl”). In some aspects, an alkyl group has 1 to 7 carbon atoms (“C₁₋₇ alkyl”). In some aspects, an alkyl group has 1 to 6 carbon atoms (“C₁₋₆ alkyl”). In some aspects, an alkyl group has 1 to 5 carbon atoms (“C₁₋₅ alkyl”). In some aspects, an alkyl group has 1 to 4 carbon atoms (“C₁₋₄ alkyl”). In some aspects, an alkyl group has 1 to 3 carbon atoms (“C₁₋₃ alkyl”). In some aspects, an alkyl group has 1 to 2 carbon atoms (“C₁₋₂ alkyl”). In some aspects, an alkyl group has 1 carbon atom (“C₁ alkyl”). In some aspects, an alkyl group has 2 to 6 carbon atoms (“C₂₋₆ alkyl”). Examples of C₁₋₆ alkyl groups include methyl (C₁), ethyl (C₂), propyl (C₃) (e.g., n-propyl, isopropyl), butyl (C₄) (e.g., n-butyl, tert-butyl, sec-butyl, isobutyl), pentyl (C₅) (e.g., n-pentyl, 3-pentanyl, amyl, neopentyl, 3-methyl-2-butanyl, tert-amyl), and hexyl (C₆) (e.g., n-hexyl). Additional examples of alkyl groups include n-heptyl (C₇), n-octyl (C₈), n-dodecyl (C₁₂), and the like. Unless otherwise specified, each instance of an alkyl group is independently unsubstituted (an “unsubstituted alkyl”) or substituted (a “substituted alkyl”) with one or more substituents (e.g., halogen, such as F). In certain aspects, the alkyl group is an unsubstituted C₁₋₁₂ alkyl (such as unsubstituted C₁₋₆ alkyl, e.g., —CH₃ (Me), unsubstituted ethyl (Et), unsubstituted propyl (Pr, e.g., unsubstituted n-propyl (n-Pr), unsubstituted isopropyl (i-Pr)), unsubstituted butyl (Bu, e.g., unsubstituted n-butyl (n-Bu), unsubstituted tert-butyl (tert-Bu or t-Bu), unsubstituted sec-butyl (sec-Bu or s-Bu), unsubstituted isobutyl (i-Bu)). In certain aspects, the alkyl group is a substituted C₁₋₁₂ alkyl (such as substituted C₁₋₆ alkyl, e.g., —CH₂F, —CHF₂, —CF₃, —CH₂CH₂F, —CH₂CHF₂, —CH₂CF₃, or benzyl (Bn)).

The term “haloalkyl” is a substituted alkyl group, wherein one or more of the hydrogen atoms are independently replaced by a halogen, e.g., fluoro, bromo, chloro, or iodo. “Perhaloalkyl” is a subset of haloalkyl, and refers to an alkyl group wherein all of the hydrogen atoms are independently replaced by a halogen, e.g., fluoro, bromo, chloro, or iodo. In some aspects, the haloalkyl moiety has 1 to 12 carbon atoms (“C₁₋₁₂ haloalkyl”). In some aspects, the haloalkyl moiety has 1 to 10 carbon atoms (“C₁₋₁₀ haloalkyl”). In some aspects, the haloalkyl moiety has 1 to 9 carbon atoms (“C₁₋₉ haloalkyl”). In some aspects, the haloalkyl moiety has 1 to 8 carbon atoms (“C₁₋₈ haloalkyl”). In some aspects, the haloalkyl moiety has 1 to 7 carbon atoms (“C₁₋₇ haloalkyl”). In some aspects, the haloalkyl moiety has 1 to 6 carbon atoms (“C₁₋₆ haloalkyl”). In some aspects, the haloalkyl moiety has 1 to 5 carbon atoms (“C₁₋₅ haloalkyl”). In some aspects, the haloalkyl moiety has 1 to 4 carbon atoms (“C₁₋₄ haloalkyl”). In some aspects, the haloalkyl moiety has 1 to 3 carbon atoms (“C₁₋₃ haloalkyl”). In some aspects, the haloalkyl moiety has 1 to 2 carbon atoms (“C₁₋₂ haloalkyl”). In some aspects, all of the haloalkyl hydrogen atoms are independently replaced with fluoro to provide a “perfluoroalkyl” group. In some aspects, all of the haloalkyl hydrogen atoms are independently replaced with chloro to provide a “perchloroalkyl” group. Examples of haloalkyl groups include —CHF₂, —CH₂F, —CF₃, —CH₂CF₃, —CF₂CF₃, —CF₂CF₂CF₃, —CCl₃, —CFCl₂, —CF₂C₁, and the like.

The term “heteroalkyl” refers to an alkyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain aspects, a heteroalkyl group refers to a saturated group having from 1 to 12 carbon atoms and 1 or more heteroatoms within the parent chain (“heteroC₁₋₁₂ alkyl”). In some aspects, a heteroalkyl group is a saturated group having 1 to 11 carbon atoms and 1 or more heteroatoms within the parent chain (“heteroC₁₋₁₁ alkyl”). In some aspects, a heteroalkyl group is a saturated group having 1 to 10 carbon atoms and 1 or more heteroatoms within the parent chain (“heteroC₁₋₁₀ alkyl”). In some aspects, a heteroalkyl group is a saturated group having 1 to 9 carbon atoms and 1 or more heteroatoms within the parent chain (“heteroC₁₋₉ alkyl”). In some aspects, a heteroalkyl group is a saturated group having 1 to 8 carbon atoms and 1 or more heteroatoms within the parent chain (“heteroC₁₋₈ alkyl”). In some aspects, a heteroalkyl group is a saturated group having 1 to 7 carbon atoms and 1 or more heteroatoms within the parent chain (“heteroC₁₋₇ alkyl”). In some aspects, a heteroalkyl group is a saturated group having 1 to 6 carbon atoms and 1 or more heteroatoms within the parent chain (“heteroC₁₋₆ alkyl”). In some aspects, a heteroalkyl group is a saturated group having 1 to 5 carbon atoms and 1 or 2 heteroatoms within the parent chain (“heteroC₁₋₅ alkyl”). In some aspects, a heteroalkyl group is a saturated group having 1 to 4 carbon atoms and 1 or 2 heteroatoms within the parent chain (“heteroC₁₋₄ alkyl”). In some aspects, a heteroalkyl group is a saturated group having 1 to 3 carbon atoms and 1 heteroatom within the parent chain (“heteroC₁₋₃ alkyl”). In some aspects, a heteroalkyl group is a saturated group having 1 to 2 carbon atoms and 1 heteroatom within the parent chain (“heteroC₁₋₂ alkyl”). In some aspects, a heteroalkyl group is a saturated group having 1 carbon atom and 1 heteroatom (“heteroC₁ alkyl”). In some aspects, a heteroalkyl group is a saturated group having 2 to 6 carbon atoms and 1 or 2 heteroatoms within the parent chain (“heteroC₂₋₆ alkyl”). Unless otherwise specified, each instance of a heteroalkyl group is independently unsubstituted (an “unsubstituted heteroalkyl”) or substituted (a “substituted heteroalkyl”) with one or more substituents. In certain aspects, the heteroalkyl group is an unsubstituted heteroC₁₋₁₂ alkyl. In certain aspects, the heteroalkyl group is a substituted heteroC₁₋₁₂ alkyl.

The term “alkenyl” refers to a radical of a straight-chain or branched hydrocarbon group having from 1 to 12 carbon atoms and one or more carbon-carbon double bonds (e.g., 1, 2, 3, or 4 double bonds). In some aspects, an alkenyl group has 1 to 12 carbon atoms (“C₁₋₁₂ alkenyl”). In some aspects, an alkenyl group has 1 to 11 carbon atoms (“C₁₋₁₁ alkenyl”). In some aspects, an alkenyl group has 1 to 10 carbon atoms (“C₁₋₁₀ alkenyl”). In some aspects, an alkenyl group has 1 to 9 carbon atoms (“C₁₋₉ alkenyl”). In some aspects, an alkenyl group has 1 to 8 carbon atoms (“C₁₋₈ alkenyl”). In some aspects, an alkenyl group has 1 to 7 carbon atoms (“C₁₋₇ alkenyl”). In some aspects, an alkenyl group has 1 to 6 carbon atoms (“C₁₋₆ alkenyl”). In some aspects, an alkenyl group has 1 to 5 carbon atoms (“C₁₋₅ alkenyl”). In some aspects, an alkenyl group has 1 to 4 carbon atoms (“C₁₋₄ alkenyl”). In some aspects, an alkenyl group has 1 to 3 carbon atoms (“C₁₋₃ alkenyl”). In some aspects, an alkenyl group has 1 to 2 carbon atoms (“C₁₋₂ alkenyl”). In some aspects, an alkenyl group has 1 carbon atom (“C₁ alkenyl”). The one or more carbon-carbon double bonds can be internal (such as in 2-butenyl) or terminal (such as in 1-butenyl). Examples of C₁₋₄ alkenyl groups include methylidenyl (C₁), ethenyl (C₂), 1-propenyl (C₃), 2-propenyl (C₃), 1-butenyl (C₄), 2-butenyl (C₄), butadienyl (C₄), and the like. Examples of C₁₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₅), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain aspects, the alkenyl group is an unsubstituted C₁₋₁₂ alkenyl. In certain aspects, the alkenyl group is a substituted C₁₋₁₂ alkenyl. In an alkenyl group, a C═C double bond for which the stereochemistry is not specified (e.g., —CH═CHCH₃ or

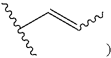

may be in the (E)- or (Z)-configuration.

The term “heteroalkenyl” refers to an alkenyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain aspects, a heteroalkenyl group refers to a group having from 1 to 12 carbon atoms, at least one double bond, and 1 or more heteroatoms within the parent chain (“heteroC₁₋₁₂ alkenyl”). In certain aspects, a heteroalkenyl group refers to a group having from 1 to 11 carbon atoms, at least one double bond, and 1 or more heteroatoms within the parent chain (“heteroC₁₋₁₁ alkenyl”). In certain aspects, a heteroalkenyl group refers to a group having from 1 to 10 carbon atoms, at least one double bond, and 1 or more heteroatoms within the parent chain (“heteroC₁₋₁₀ alkenyl”). In some aspects, a heteroalkenyl group has 1 to 9 carbon atoms at least one double bond, and 1 or more heteroatoms within the parent chain (“heteroC₁₋₉ alkenyl”). In some aspects, a heteroalkenyl group has 1 to 8 carbon atoms, at least one double bond, and 1 or more heteroatoms within the parent chain (“heteroC₁₋₈ alkenyl”). In some aspects, a heteroalkenyl group has 1 to 7 carbon atoms, at least one double bond, and 1 or more heteroatoms within the parent chain (“heteroC₁₋₇ alkenyl”). In some aspects, a heteroalkenyl group has 1 to 6 carbon atoms, at least one double bond, and 1 or more heteroatoms within the parent chain (“heteroC₁₋₆ alkenyl”). In some aspects, a heteroalkenyl group has 1 to 5 carbon atoms, at least one double bond, and 1 or 2 heteroatoms within the parent chain (“heteroC₁₋₅ alkenyl”). In some aspects, a heteroalkenyl group has 1 to 4 carbon atoms, at least one double bond, and 1 or 2 heteroatoms within the parent chain (“heteroC₁₋₄ alkenyl”). In some aspects, a heteroalkenyl group has 1 to 3 carbon atoms, at least one double bond, and 1 heteroatom within the parent chain (“heteroC₁₋₃ alkenyl”). In some aspects, a heteroalkenyl group has 1 to 2 carbon atoms, at least one double bond, and 1 heteroatom within the parent chain (“heteroC₁₋₂ alkenyl”). In some aspects, a heteroalkenyl group has 1 to 6 carbon atoms, at least one double bond, and 1 or 2 heteroatoms within the parent chain (“heteroC₁₋₆ alkenyl”).

Unless otherwise specified, each instance of a heteroalkenyl group is independently unsubstituted (an “unsubstituted heteroalkenyl”) or substituted (a “substituted heteroalkenyl”) with one or more substituents. In certain aspects, the heteroalkenyl group is an unsubstituted heteroC₁₋₂₀ alkenyl. In certain aspects, the heteroalkenyl group is a substituted heteroC₁₋₂₀ alkenyl.

The term “alkynyl” refers to a radical of a straight-chain or branched hydrocarbon group having from 1 to 10 carbon atoms (“C₁₋₁₀ alkynyl”). In some aspects, an alkynyl group has 1 to 9 carbon atoms (“C₁₋₉ alkynyl”). In some aspects, an alkynyl group has 1 to 8 carbon atoms (“C₁₋₈ alkynyl”). In some aspects, an alkynyl group has 1 to 7 carbon atoms (“C₁₋₇ alkynyl”). In some aspects, an alkynyl group has 1 to 6 carbon atoms (“C₁₋₆ alkynyl”). In some aspects, an alkynyl group has 1 to 5 carbon atoms (“C₁₋₅ alkynyl”). In some aspects, an alkynyl group has 1 to 4 carbon atoms (“C₁₋₄ alkynyl”). In some aspects, an alkynyl group has 1 to 3 carbon atoms (“C₁₋₃ alkynyl”). In some aspects, an alkynyl group has 1 to 2 carbon atoms (“C₁₋₂ alkynyl”). In some aspects, an alkynyl group has 1 carbon atom (“C₁ alkynyl”). The one or more carbon-carbon triple bonds can be internal (such as in 2-butynyl) or terminal (such as in 1-butynyl). Examples of C₁₋₄ alkynyl groups include, without limitation, methylidynyl (C₁), ethynyl (C₂), 1-propynyl (C₃), 2-propynyl (C₃), 1-butynyl (C₄), 2-butynyl (C₄), and the like. Examples of C₁₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents.

The term “heteroalkynyl” refers to an alkynyl group, which further includes at least one heteroatom (e.g., 1, 2, 3, or 4 heteroatoms) selected from oxygen, nitrogen, or sulfur within (e.g., inserted between adjacent carbon atoms of) and/or placed at one or more terminal position(s) of the parent chain. In certain aspects, a heteroalkynyl group refers to a group having from 1 to 10 carbon atoms, at least one triple bond, and 1 or more heteroatoms within the parent chain (“heteroC₁₋₁₀ alkynyl”). In some aspects, a heteroalkynyl group has 1 to 9 carbon atoms, at least one triple bond, and 1 or more heteroatoms within the parent chain (“heteroC₁₋₉ alkynyl”). In some aspects, a heteroalkynyl group has 1 to 8 carbon atoms, at least one triple bond, and 1 or more heteroatoms within the parent chain (“heteroC₁-s alkynyl”). In some aspects, a heteroalkynyl group has 1 to 7 carbon atoms, at least one triple bond, and 1 or more heteroatoms within the parent chain (“heteroC₁₋₇ alkynyl”). In some aspects, a heteroalkynyl group has 1 to 6 carbon atoms, at least one triple bond, and 1 or more heteroatoms within the parent chain (“heteroC₁₋₆ alkynyl”). In some aspects, a heteroalkynyl group has 1 to 5 carbon atoms, at least one triple bond, and 1 or 2 heteroatoms within the parent chain (“heteroC₁₋₅ alkynyl”). In some aspects, a heteroalkynyl group has 1 to 4 carbon atoms, at least one triple bond, and 1 or 2 heteroatoms within the parent chain (“heteroC₁₋₄ alkynyl”). In some aspects, a heteroalkynyl group has 1 to 3 carbon atoms, at least one triple bond, and 1 heteroatom within the parent chain (“heteroC₁₋₃ alkynyl”). In some aspects, a heteroalkynyl group has 1 to 2 carbon atoms, at least one triple bond, and 1 heteroatom within the parent chain (“heteroC₁₋₂ alkynyl”). In some aspects, a heteroalkynyl group has 1 to 6 carbon atoms, at least one triple bond, and 1 or 2 heteroatoms within the parent chain (“heteroC₁₋₆ alkynyl”). Unless otherwise specified, each instance of a heteroalkynyl group is independently unsubstituted (an “unsubstituted heteroalkynyl”) or substituted (a “substituted heteroalkynyl”) with one or more substituents.

The term “carbocyclyl” or “carbocyclic” refers to a radical of a non-aromatic cyclic hydrocarbon group having from 3 to 10 ring carbon atoms (“C₃₋₁₀ carbocyclyl”) and zero heteroatoms in the non-aromatic ring system. In some aspects, a carbocyclyl group has 3 to 10 ring carbon atoms (“C₃₋₁₀ carbocyclyl”). In some aspects, a carbocyclyl group has 3 to 8 ring carbon atoms (“C₃₋₈ carbocyclyl”). In some aspects, a carbocyclyl group has 3 to 7 ring carbon atoms (“C₃₋₇ carbocyclyl”). In some aspects, a carbocyclyl group has 3 to 6 ring carbon atoms (“C₃₋₆ carbocyclyl”). In some aspects, a carbocyclyl group has 4 to 6 ring carbon atoms (“C₄₋₆ carbocyclyl”). In some aspects, a carbocyclyl group has 5 to 6 ring carbon atoms (“C₅₋₆ carbocyclyl”). In some aspects, a carbocyclyl group has 5 to 10 ring carbon atoms (“C₅₋₁₀ carbocyclyl”). Exemplary C₃₋₆ carbocyclyl groups include cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₈), cyclooctenyl (C₈), bicyclo[2.2.1]heptanyl (C₇), bicyclo[2.2.2]octanyl (C₈), and the like. Exemplary C₃₋₁₀ carbocyclyl groups include the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. Exemplary C₃₋₈ carbocyclyl groups include the aforementioned C₃₋₁₀ carbocyclyl groups, and the like. As the foregoing examples illustrate, in certain aspects, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain aspects, the carbocyclyl group is an unsubstituted C₃₋₁₀ carbocyclyl. In certain aspects, the carbocyclyl group is a substituted C₃₋₁₀ carbocyclyl. In some aspects, a cycloalkyl group has 3 to 10 ring carbon atoms (“C₃₋₁₀ cycloalkyl”). In some aspects, a cycloalkyl group has 3 to 8 ring carbon atoms (“C₃₋₈ cycloalkyl”). In some aspects, a cycloalkyl group has 3 to 6 ring carbon atoms (“C₃₋₆ cycloalkyl”). In some aspects, a cycloalkyl group has 4 to 6 ring carbon atoms (“C₄₋₆ cycloalkyl”). In some aspects, a cycloalkyl group has 5 to 6 ring carbon atoms (“C₅₋₆ cycloalkyl”). In some aspects, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain aspects, the cycloalkyl group is an unsubstituted C₃₋₁₄ cycloalkyl. In certain aspects, the cycloalkyl group is a substituted C₃₋₁₄ cycloalkyl. In certain aspects, the carbocyclyl includes 0, 1, or 2 C═C double bonds in the carbocyclic ring system, as valency permits.

The term “heterocyclyl” or “heterocyclic” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain aspects, the heterocyclyl group is an unsubstituted 3-14 membered heterocyclyl. In certain aspects, the heterocyclyl group is a substituted 3-14 membered heterocyclyl. In certain aspects, the heterocyclyl is substituted or unsubstituted, 3- to 7-membered, monocyclic heterocyclyl, wherein 1, 2, or 3 atoms in the heterocyclic ring system are independently oxygen, nitrogen, or sulfur, as valency permits.

In some aspects, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some aspects, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some aspects, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some aspects, the 5-6 membered heterocyclyl has 1-3 ring heteroatoms selected from nitrogen, oxygen, and sulfur. In some aspects, the 5-6 membered heterocyclyl has 1-2 ring heteroatoms selected from nitrogen, oxygen, and sulfur. In some aspects, the 5-6 membered heterocyclyl has 1 ring heteroatom selected from nitrogen, oxygen, and sulfur.

Exemplary 3-membered heterocyclyl groups containing 1 heteroatom include azirdinyl, oxiranyl, and thiiranyl. Exemplary 4-membered heterocyclyl groups containing 1 heteroatom include azetidinyl, oxetanyl, and thietanyl. Exemplary 5-membered heterocyclyl groups containing 1 heteroatom include tetrahydrofuranyl, dihydrofuranyl, tetrahydrothiophenyl, dihydrothiophenyl, pyrrolidinyl, dihydropyrrolyl, and pyrrolyl-2,5-dione. Exemplary 5-membered heterocyclyl groups containing 2 heteroatoms include dioxolanyl, oxathiolanyl and dithiolanyl. Exemplary 5-membered heterocyclyl groups containing 3 heteroatoms include triazolinyl, oxadiazolinyl, and thiadiazolinyl. Exemplary 6-membered heterocyclyl groups containing 1 heteroatom include piperidinyl, tetrahydropyranyl, dihydropyridinyl, and thianyl. Exemplary 6-membered heterocyclyl groups containing 2 heteroatoms include piperazinyl, morpholinyl, dithianyl, and dioxanyl. Exemplary 6-membered heterocyclyl groups containing 3 heteroatoms include triazinyl. Exemplary 7-membered heterocyclyl groups containing 1 heteroatom include azepanyl, oxepanyl and thiepanyl. Exemplary 8-membered heterocyclyl groups containing 1 heteroatom include azocanyl, oxecanyl and thiocanyl. Exemplary bicyclic heterocyclyl groups include indolinyl, isoindolinyl, dihydrobenzofuranyl, dihydrobenzothienyl, tetra-hydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

The term “aryl” refers to a radical of a monocyclic or polycyclic (e.g., bicyclic or tricyclic) 4n+2 aromatic ring system (e.g., having 6, 10, or 14 π electrons shared in a cyclic array) having 6-14 ring carbon atoms and zero heteroatoms provided in the aromatic ring system (“C₆₋₁₄ aryl”). In some aspects, an aryl group has 6 ring carbon atoms (“C₆ aryl”; e.g., phenyl). In some aspects, an aryl group has 10 ring carbon atoms (“C₁₀ aryl”; e.g., naphthyl such as 1-naphthyl and 2-naphthyl). In some aspects, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain aspects, the aryl group is an unsubstituted C₆₋₁₄ aryl. In certain aspects, the aryl group is a substituted C₆₋₁₄ aryl.

“Aralkyl” is a subset of “alkyl” and refers to an alkyl group substituted by an aryl group, wherein the point of attachment is on the alkyl moiety.

The term “heteroaryl” refers to a radical of a 5-14 membered monocyclic or polycyclic (e.g., bicyclic, tricyclic) 4n+2 aromatic ring system (e.g., having 6, 10, or 14 n electrons shared in a cyclic array) having ring carbon atoms and 1-4 ring heteroatoms provided in the aromatic ring system, wherein each heteroatom is independently selected from nitrogen, oxygen, and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, e.g., either the ring bearing a heteroatom (e.g., 2-indolyl) or the ring that does not contain a heteroatom (e.g., 5-indolyl). In certain aspects, the heteroaryl is substituted or unsubstituted, 5- or 6-membered, monocyclic heteroaryl, wherein 1, 2, 3, or 4 atoms in the heteroaryl ring system are independently oxygen, nitrogen, or sulfur. In certain aspects, the heteroaryl is substituted or unsubstituted, 9- or 10-membered, bicyclic heteroaryl, wherein 1, 2, 3, or 4 atoms in the heteroaryl ring system are independently oxygen, nitrogen, or sulfur.

In some aspects, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some aspects, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some aspects, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some aspects, the 5-6 membered heteroaryl has 1-3 ring heteroatoms selected from nitrogen, oxygen, and sulfur. In some aspects, the 5-6 membered heteroaryl has 1-2 ring heteroatoms selected from nitrogen, oxygen, and sulfur. In some aspects, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain aspects, the heteroaryl group is an unsubstituted 5-14 membered heteroaryl. In certain aspects, the heteroaryl group is a substituted 5-14 membered heteroaryl.

Exemplary 5-membered heteroaryl groups containing 1 heteroatom include pyrrolyl, furanyl, and thiophenyl. Exemplary 5-membered heteroaryl groups containing 2 heteroatoms include imidazolyl, pyrazolyl, oxazolyl, isoxazolyl, thiazolyl, and isothiazolyl. Exemplary 5-membered heteroaryl groups containing 3 heteroatoms include triazolyl, oxadiazolyl, and thiadiazolyl. Exemplary 5-membered heteroaryl groups containing 4 heteroatoms include tetrazolyl. Exemplary 6-membered heteroaryl groups containing 1 heteroatom include pyridinyl. Exemplary 6-membered heteroaryl groups containing 2 heteroatoms include pyridazinyl, pyrimidinyl, and pyrazinyl. Exemplary 6-membered heteroaryl groups containing 3 or 4 heteroatoms include triazinyl and tetrazinyl, respectively. Exemplary 7-membered heteroaryl groups containing 1 heteroatom include azepinyl, oxepinyl, and thiepinyl. Exemplary 5,6-bicyclic heteroaryl groups include indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include naphthyridinyl, pteridinyl, quinolinyl, isoquinolinyl, cinnolinyl, quinoxalinyl, phthalazinyl, and quinazolinyl. Exemplary tricyclic heteroaryl groups include phenanthridinyl, dibenzofuranyl, carbazolyl, acridinyl, phenothiazinyl, phenoxazinyl, and phenazinyl.

“Heteroaralkyl” is a subset of “alkyl” and refers to an alkyl group substituted by a heteroaryl group, wherein the point of attachment is on the alkyl moiety.

The term “unsaturated bond” refers to a double or triple bond.

The term “unsaturated” or “partially unsaturated” refers to a moiety that includes at least one double or triple bond.

The term “saturated” or “fully saturated” refers to a moiety that does not contain a double or triple bond, e.g., the moiety only contains single bonds.

Affixing the suffix “-ene” to a group indicates the group is a divalent moiety, e.g., alkylene is the divalent moiety of alkyl, alkenylene is the divalent moiety of alkenyl, alkynylene is the divalent moiety of alkynyl, heteroalkylene is the divalent moiety of heteroalkyl, heteroalkenylene is the divalent moiety of heteroalkenyl, heteroalkynylene is the divalent moiety of heteroalkynyl, carbocyclylene is the divalent moiety of carbocyclyl, heterocyclylene is the divalent moiety of heterocyclyl, arylene is the divalent moiety of aryl, and heteroarylene is the divalent moiety of heteroaryl.

A group is optionally substituted unless expressly provided otherwise. The term “optionally substituted” refers to being substituted or unsubstituted. In certain aspects, alkyl, alkenyl, alkynyl, heteroalkyl, heteroalkenyl, heteroalkynyl, carbocyclyl, heterocyclyl, aryl, and heteroaryl groups are optionally substituted. “Optionally substituted” refers to a group which may be substituted or unsubstituted (e.g., “substituted” or “unsubstituted” alkyl, “substituted” or “unsubstituted” alkenyl, “substituted” or “unsubstituted” alkynyl, “substituted” or “unsubstituted” heteroalkyl, “substituted” or “unsubstituted” heteroalkenyl, “substituted” or “unsubstituted” heteroalkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted” means that at least one hydrogen present on a group is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, and includes any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety. The invention is not intended to be limited in any manner by the exemplary substituents described herein.

Exemplary carbon atom substituents include halogen, —CN, —NO₂, —N₃, —SO₂H, —SO₃H, —OH, —OR^{aa}, —ON(R^{bb})₂, —N(R^{bb})₂, —N(R^{bb})₃⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)(R^{cc})₂, —P(═O)(OR^{cc})₂, —OP(═O)(R^{aa})₂, —OP(═O)(OR^{cc})₂, —P(═O)(N(R^{bb})₂)₂, —OP(═O)(N(R^{bb})₂)₂, —NR^{bb}P(═O)(R^{aa})₂, —NR^{bb}P(═O)(OR^{cc})₂, —NR^{bb}P(═O)(N(R^{bb})₂)₂, —P(R^{cc})₂, —P(OR^{cc})₂, —P(R^{cc})₃⁺X⁻, —P(OR^{cc})₃⁺X⁻, —P(R^{cc})₄, —P(OR^{cc})₄, —OP(R^{cc})₂, —OP(R^{cc})₃⁺X⁻, —OP(OR^{cc})₂, —OP(OR^{cc})₃⁺X⁻, —OP(R^{cc})₄, —OP(OR^{cc})₄, —B(R^{aa})₂, —B(OR^{cc})₂, —BR^{aa}(OR^{cc}), C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups; wherein X⁻ is a counterion;

or two geminal hydrogens on a carbon atom are replaced with the group ═O, ═S, ═NN(R^{bb})₂, ═NNR^{bb}C(═O)R—, ═NNR^{bb}C(═O)OR^{aa}, ═NNR^{bb}S(═O)₂R^{aa}, ═NR^{bb}, or ═NOR^{cc};

each instance of R^{aa} is, independently, selected from C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each of the alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)(R^{aa})₂, —P(═O)(OR^{cc})₂, —P(═O)(N(R^{cc})₂)₂, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀alkenyl, heteroC₁₋₂₀alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{cc} is, independently, selected from hydrogen, C₁₋₂₀ alkyl, C₁₋₂₀ perhaloalkyl, C₁₋₂₀ alkenyl, C₁₋₂₀ alkynyl, heteroC₁₋₂₀ alkyl, heteroC₁₋₂₀ alkenyl, heteroC₁₋₂₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{dd} groups;

each instance of R^{dd} is, independently, selected from halogen, —CN, —NO₂, —N₃, —SO₂H, —SO₃H, —OH, —OR^{ee}, —ON(R^{ff})₂, —N(R^{f})₂, —N(R^{ff})₃⁺X⁻, —N(OR^{ee})R^{ff}, —SH, —SR^{ee}, —SSR^{ee}, —C(═O)R^{ee}, —CO₂H, —CO₂R^{ee}, —OC(═O)R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)(OR^{ee})₂, —P(═O)(R^{ee})₂, —OP(═O)(R^{ee})₂, —OP(═O)(OR^{ee})₂, C₁₋₁₀ alkyl, C₁₋₁₀ perhaloalkyl, C₁₋₁₀ alkenyl, C₁₋₁₀ alkynyl, heteroC₁₋₁₀alkyl, heteroC₁₋₁₀alkenyl, heteroC₁₋₁₀alkynyl, C₃₋₁₀ carbocyclyl, 3-10 membered heterocyclyl, C₆₋₁₀ aryl, 5-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; wherein X⁻ is a counterion;

each instance of R^{ee} is, independently, selected from C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{ff} is, independently, selected from hydrogen, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, 3-10 membered heterocyclyl, C₆₋₁₀ aryl and 5-10 membered heteroaryl, or two R^{ff} groups are joined to form a 3-10 membered heterocyclyl or 5-10 membered heteroaryl ring, wherein each alkyl, alkenyl, alkynyl, heteroalkyl, heteroalkenyl, heteroalkynyl, carbocyclyl, heterocyclyl, aryl, and heteroaryl is independently substituted with 0, 1, 2, 3, 4, or 5 R^{gg} groups;

each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃⁺X⁻, —NH(C₁₋₆ alkyl)₂⁺X⁻, —NH₂(C₁₋₆ alkyl)⁺X⁻, —NH₃⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(═O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)(OC₁₋₆ alkyl)₂, —P(═O)(C₁₋₆ alkyl)₂, —OP(═O)(C₁₋₆ alkyl)₂, —OP(═O)(OC₁₋₆ alkyl)₂, C₁₋₁₀ alkyl, C₁₋₁₀ perhaloalkyl, C₁₋₁₀ alkenyl, C₁₋₁₀ alkynyl, heteroC₁₋₁₀ alkyl, heteroC₁₋₁₀ alkenyl, heteroC₁₋₁₀ alkynyl, C₃₋₁₀ carbocyclyl, C₆₋₁₀ aryl, 3-10 membered heterocyclyl, or 5-10 membered heteroaryl; or two geminal R⁹⁹ substituents can be joined to form ═O or ═S; and

each X⁻ is a counterion.

In certain aspects, the carbon atom substituents are independently halogen, substituted (e.g., substituted with one or more halogen) or unsubstituted C₁₋₆ alkyl, —OR^{aa}, SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂. In certain aspects, the carbon atom substituents are independently halogen, substituted (e.g., substituted with one or more halogen) or unsubstituted C₁₋₁₀ alkyl, —OR^{aa}, —SR^{aa}, —N(R^{bb})₂, —CN, —SCN, —NO₂, —C(═O)R^{aa}, —CO₂R^{aa}, —C(═O)N(R^{bb})₂, —OC(═O)R^{aa}, —OCO₂R^{aa}, —OC(═O)N(R^{bb})₂, —NR^{bb}C(═O)R^{aa}, —NR^{bb}CO₂R^{aa}, or —NR^{bb}C(═O)N(R^{bb})₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts). In certain aspects, the carbon atom substituents are independently halogen, substituted (e.g., substituted with one or more halogen) or unsubstituted C₁₋₆ alkyl, —OR^{aa}, —SR^{aa}, —N(R^{bb})₂, —CN, —SCN, or —NO₂. In certain aspects, the carbon atom substituents are independently halogen, substituted (e.g., substituted with one or more halogen moieties) or unsubstituted C₁₋₁₀ alkyl, —OR^{aa}, —SR^{aa}, —N(R^{bb})₂, —CN, —SCN, or —NO₂, wherein R^{aa} is hydrogen, substituted (e.g., substituted with one or more halogen) or unsubstituted C₁₋₁₀ alkyl, an oxygen protecting group (e.g., silyl, TBDPS, TBDMS, TIPS, TES, TMS, MOM, THP, t-Bu, Bn, allyl, acetyl, pivaloyl, or benzoyl) when attached to an oxygen atom, or a sulfur protecting group (e.g., acetamidomethyl, t-Bu, 3-nitro-2-pyridine sulfenyl, 2-pyridine-sulfenyl, or triphenylmethyl) when attached to a sulfur atom; and each R^{bb} is independently hydrogen, substituted (e.g., substituted with one or more halogen) or unsubstituted C₁₋₁₀ alkyl, or a nitrogen protecting group (e.g., Bn, Boc, Cbz, Fmoc, trifluoroacetyl, triphenylmethyl, acetyl, or Ts).

In certain aspects, the molecular weight of a carbon atom substituent is lower than 250, lower than 200, lower than 150, lower than 100, or lower than 50 g/mol. In certain aspects, a carbon atom substituent consists of carbon, hydrogen, fluorine, chlorine, bromine, iodine, oxygen, sulfur, nitrogen, and/or silicon atoms. In certain aspects, a carbon atom substituent consists of carbon, hydrogen, fluorine, chlorine, bromine, iodine, oxygen, sulfur, and/or nitrogen atoms. In certain aspects, a carbon atom substituent consists of carbon, hydrogen, fluorine, chlorine, bromine, and/or iodine atoms. In certain aspects, a carbon atom substituent consists of carbon, hydrogen, fluorine, and/or chlorine atoms.

The term “halo” or “halogen” refers to fluorine (fluoro, —F), chlorine (chloro, —Cl), bromine (bromo, —Br), or iodine (iodo, —I).

The term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR—, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃⁺X⁻, —OP(OR^{cc})₂, —OP(OR^{cc})₃⁺X⁻, —OP(═O)(R^{cc})₂, —OP(═O)(OR^{cc})₂, and —OP(═O)(N(R^{bb}))₂, wherein X⁻, R^{aa}, R^{bb}, and R^{cc} are as defined herein.

The term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═S)OR^{aa}, —SC(═S) N(R^{bb})₂, —SC(═O)SR^{aa}, —SC(═O)OR^{aa}, —SC(═O)N(R^{bb})₂, and —SC(═O)R^{aa}, wherein R^{aa} and R^{cc} are as defined herein.

The term “amino” refers to the group —NH₂. The term “substituted amino,” by extension, refers to a monosubstituted amino, a disubstituted amino, or a trisubstituted amino. In certain aspects, the “substituted amino” is a monosubstituted amino or a disubstituted amino group.

The term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R—, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(N(R^{bb})₂)₂, wherein R^{aa}, R^{bb} and R^{cc} are as defined herein, and wherein R^{bb} of the group —NH(R^{bb}) is not hydrogen.

The term “disubstituted amino” refers to an amino group wherein the nitrogen atom directly attached to the parent molecule is substituted with two groups other than hydrogen, and includes groups selected from —N(R^{bb})₂, —NR^{bb} C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(N(R^{bb})₂)₂, wherein R^{aa}, R^{bb}, and R^{cc} are as defined herein, with the proviso that the nitrogen atom directly attached to the parent molecule is not substituted with hydrogen.

The term “trisubstituted amino” refers to an amino group wherein the nitrogen atom directly attached to the parent molecule is substituted with three groups, and includes groups selected from —N(R^{bb})₃ and —N(R^{bb})₃⁺X⁻, wherein R^{bb} and X⁻ are as defined herein.

The term “sulfonyl” refers to a group selected from —SO₂N(R^{bb})₂, —SO₂R^{aa}, and —SO₂OR^{aa}, wherein R^{aa} and R^{bb} are as defined herein.

The term “sulfinyl” refers to the group —S(═O)R^{aa}, wherein R^{aa} is as defined herein.

The term “acyl” refers to a group having the general formula —C(═O)R^{X1}, —C(═O)OR^{X1}, —C(═O)—O—C(═O)R^{X1}, —C(═O)SR^{X1}, —C(═O)N(R^{X1})₂, —C(═S)R^{X1}, —C(═S)N(R^{X1})₂, and —C(═S)S(R^{X1}), —C(═NR^{X1})R^{X1}, —C(═NRxi)OR^{X1}, —C(═NR^{X1})SR^{X1}, and —C(═NR^{X1})N(R^{X1})₂, wherein R^{X1} is hydrogen; halogen; substituted or unsubstituted hydroxyl; substituted or unsubstituted thiol; substituted or unsubstituted amino; substituted or unsubstituted acyl, cyclic or acyclic, substituted or unsubstituted, branched or unbranched aliphatic; cyclic or acyclic, substituted or unsubstituted, branched or unbranched heteroaliphatic; cyclic or acyclic, substituted or unsubstituted, branched or unbranched alkyl; cyclic or acyclic, substituted or unsubstituted, branched or unbranched alkenyl; substituted or unsubstituted alkynyl; substituted or unsubstituted aryl, substituted or unsubstituted heteroaryl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, mono- or di-aliphaticamino, mono- or di-heteroaliphaticamino, mono- or di-alkylamino, mono- or di-heteroalkylamino, mono- or di-arylamino, or mono- or di-heteroarylamino; or two R^{X1} groups taken together form a 5- to 6-membered heterocyclic ring. Exemplary acyl groups include aldehydes (—CHO), carboxylic acids (—CO₂H), ketones, acyl halides, esters, amides, imines, carbonates, carbamates, and ureas. Acyl substituents include, but are not limited to, any of the substituents described herein, that result in the formation of a stable moiety (e.g., aliphatic, alkyl, alkenyl, alkynyl, heteroaliphatic, heterocyclic, aryl, heteroaryl, acyl, oxo, imino, thiooxo, cyano, isocyano, amino, azido, nitro, hydroxyl, thiol, halo, aliphaticamino, heteroaliphaticamino, alkylamino, heteroalkylamino, arylamino, heteroarylamino, alkylaryl, arylalkyl, aliphaticoxy, heteroaliphaticoxy, alkyloxy, heteroalkyloxy, aryloxy, heteroaryloxy, aliphaticthioxy, heteroaliphaticthioxy, alkylthioxy, heteroalkylthioxy, arylthioxy, heteroarylthioxy, acyloxy, and the like, each of which may or may not be further substituted).

The term “carbonyl” refers to a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the terms “salt” or “salts” refers to an acid addition or base addition salt of a compound of the invention. “Salts” include in particular “pharmaceutically acceptable salts.”

The term “pharmaceutically acceptable salts” refers to salts that retain the biological effectiveness and properties of the compounds of this invention and, which typically are not biologically or otherwise undesirable. In many cases, the compounds of the present invention are capable of forming acid and/or base salts by virtue of the presence of amino and/or carboxyl groups or groups similar thereto.

Pharmaceutically acceptable acid addition salts can be formed with inorganic acids and organic acids, e.g., acetate, aspartate, benzoate, besylate, bromide/hydrobromide, bicarbonate/carbonate, bisulfate/sulfate, camphorsulformate, chloride/hydrochloride, chlortheophyllonate, citrate, ethandisulfonate, fumarate, gluceptate, gluconate, glucuronate, hippurate, hydroiodide/iodide, isethionate, lactate, lactobionate, laurylsulfate, malate, maleate, malonate, mandelate, mesylate, methylsulphate, naphthoate, napsylate, nicotinate, nitrate, octadecanoate, oleate, oxalate, palmitate, pamoate, phosphate/hydrogen phosphate/dihydrogen phosphate, polygalacturonate, propionate, Stearate. Succinate, Sulfosalicylate, tartrate, tosylate and trifluoroacetate salts.

Inorganic acids from which salts can be derived include, for example, hydrochloric acid, hydrobromic acid, sulfuric acid, nitric acid, phosphoric acid, and the like.

Organic acids from which salts can be derived include, for example, acetic acid, propionic acid, glycolic acid, oxalic acid, maleic acid, malonic acid, Succinic acid, fumaric acid, tartaric acid, citric acid, benzoic acid, mandelic acid, methanesulfonic acid, ethanesulfonic acid, toluenesulfonic acid, sulfosalicylic acid, and the like. Pharmaceutically acceptable base addition salts can be formed with inorganic and organic bases.

Inorganic bases from which salts can be derived include, for example, ammonium salts and metals from columns I to XII of the periodic table. In certain aspects, the salts are derived from Sodium, potassium, ammonium, calcium, magnesium, iron, silver, Zinc, and copper, particularly suitable salts include ammonium, potassium, Sodium, calcium and magnesium salts.

Organic bases from which salts can be derived include, for example, primary, secondary, and tertiary amines, substituted amines including naturally occurring substituted amines, cyclic amines, basic ion exchange resins, and the like. Certain organic amines include isopropylamine, benzathine, cholinate, diethanolamine, diethylamine, lysine, meglumine, pip erazine and tromethamine.

The pharmaceutically acceptable salts of the present disclosure can be synthesized from a parent compound, a basic or acidic moiety, by conventional chemical methods. Generally, such salts can be prepared by reacting free acid forms of these compounds with a stoichiometric amount of the appropriate base (such as Na, Ca, Mg, or Khydroxide, carbonate, bicarbonate or the like), or by reacting free base forms of these compounds with a stoichiometric amount of the appropriate acid. Such reactions are typically carried out in water or in an organic solvent, or in a mixture of the two. Generally, use of non-aqueous media like ether, ethyl acetate, ethanol, isopropanol, or acetonitrile is desirable, where practicable. Lists of additional Suitable salts can be found, e.g., in “Remington's Pharmaceutical Sciences”, 20th ed., Mack Publishing Company, Easton, Pa., (1985); and in “Handbook of Pharmaceutical Salts: Properties, Selection, and Use” by Stahl and Wermuth (Wiley-VCH. Weinheim, Germany, 2002).

Recombinant Nucleic Acids that Encode SMN1

In some aspects, a combined therapy for treating SMA includes administration (e.g., concurrently or sequentially) of a recombinant nucleic acid that encodes SMN1 (e.g., administered in a viral vector, such as an rAAV) in addition to other therapies described herein (e.g., a SMN2 ASO or a small molecule that increases SMN function). In some aspects, a recombinant nucleic acid that encodes SMN1 (also referred to herein as a recombinant SMN1 gene) comprises an SMN1 gene operatively linked to a promoter (e.g., to a promoter that is active in motor neuron cells). In some aspects, a recombinant nucleic acid that encodes SMN1 is provided in a non-viral vector (e.g., in a non-viral plasmid). However, in some aspects, a recombinant nucleic acid that encodes SMN1 is provided in a recombinant viral vector (e.g., in a recombinant viral genome packaged within a viral capsid). In some aspects, the recombinant SMN1 gene is provided in a recombinant adeno-associated viral (rAAV) genome and packaged within an AAV capsid particle.

In some aspects a recombinant SMN1 gene is administered to a subject in a viral vector. In some aspects, the recombinant SMN1 gene is administered in a recombinant AAV genome comprising flanking AAV inverted terminal repeats (ITRs). Accordingly, in some aspects a recombinant viral particle (e.g., an rAAV particle) comprising a gene that encodes SMN1 is administered to a subject along with a SMN2 ASO.

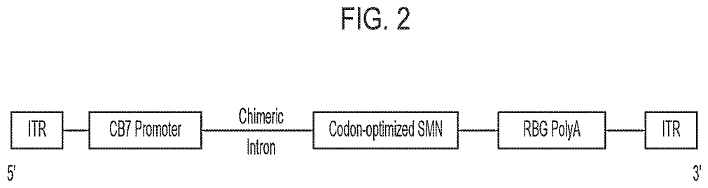

FIG. 2 provides a non-limiting example of a recombinant viral genome that comprises an SMN1 gene operably linked to a promoter. FIG. 2 illustrates an SMN1 gene flanked by AAV ITRs. The SMN1 gene comprises a human SMN1 codon optimized SMN1 open reading frame and is operably linked to a CB7 promoter (chicken beta actin promoter with a cytomegalovirus (CMV) enhancer). The recombinant AAV genome also comprises a chicken beta-actin intron, and a rabbit beta-globin poly A signal. The rAAV genome illustrated in FIG. 2 is non-limiting and alternative SMN1 coding sequences, promoters, and other regulatory elements can be used.

In some aspects, the rAAV genome is packaged in a viral capsid. In some aspects, the capsid proteins are hu68 serotype capsid proteins. However, other capsid proteins of other serotypes can be used.

These and other aspects of the recombinant SMN1 gene are described in more detail in the following paragraphs.

SMN1 coding sequences:

In some aspects, a coding sequence that encodes a wild-type human SMN protein (e.g., SMN1 cDNA sequence) is provided. Nucleic acid sequences encoding the human SMN1 are known in the art. See, e.g., GenBank Accession Nos. NM_001297715.1; NM_000344.3; NM_022874.2, DQ894095, NM_000344, NM_022874, and BC062723 for non-limiting examples of nucleic acid sequences of human SMN1. A non-limiting example of an amino acid sequence for wild-type human SMN protein is provided in UniProtKB/Swiss-Prot: Q16637.1. Other publications describing SMN1 coding sequence are, see, e.g., WO2010129021A1, and WO2009151546A2, the entire contents of which are incorporated herein by reference.

In some aspects, a coding sequence that encodes a functional SMN protein is provided. In some aspects, the amino acid sequence of the functional SMN1 is that of a human SMN1 protein or a sequence sharing 95% identity therewith.

In some aspects, a modified hSMN1 coding sequence is provided. In some aspects, the modified hSMN1 coding sequence has less than about 80% identity, preferably about 75% identity or less to a full-length native hSMN1 coding sequence. In some aspects, the modified hSMN1 coding sequence is characterized by an improved translation rate as compared to native hSMN1 following AAV-mediated delivery (e.g., using an rAAV particle). In some aspects, the modified hSMN1 coding sequence shares less than about 80%, 79%, 78%, 77%, 76%, 75%, 74%, 73%, 72%, 71%, 70%, 69%, 68%, 67%, 66%, 65%, 64%, 63%, 62%, 61% or less identity to a full length native hSMN1 coding sequence.

The term “percent (%) identity”, “sequence identity”, “percent sequence identity”, or “percent identical” in the context of nucleic acid sequences refers to the residues in the two sequences which are the same when aligned for correspondence. The length of sequence identity comparison may be over the full-length of the genome, the full-length of a gene coding sequence, or a fragment of at least about 500 to 5000 nucleotides, is desired. However, identity among smaller fragments, e.g., of at least about nine nucleotides, usually at least about 20 to 24 nucleotides, at least about 28 to 32 nucleotides, at least about 36 or more nucleotides, may also be desired.

“Aligned” sequences or “alignments” refer to multiple nucleic acid sequences or protein (amino acids) sequences, often containing corrections for missing or additional bases or amino acids as compared to a reference sequence.

Alignments can be performed using any of a variety of publicly or commercially available Multiple Sequence Alignment Programs. Sequence alignment programs are available for amino acid sequences, e.g., the “Clustal X”, “MAP”, “PIMA”, “MSA”, “BLOCKMAKER”, “MEME”, and “Match-Box” programs. Generally, any of these programs are used at default settings, although one of skill in the art can alter these settings as needed. Alternatively, one of skill in the art can utilize another algorithm or computer program which provides at least the level of identity or alignment as that provided by the referenced algorithms and programs. See, e.g., J. D. Thomson et al, Nucl. Acids. Res., “A comprehensive comparison of multiple sequence alignments”, 27(13):2682-2690 (1999).

Multiple sequence alignment programs are also available for nucleic acid sequences. Examples of such programs include, “Clustal W”, “CAP Sequence Assembly”, “BLAST”, “MAP”, and “MEME”, which are accessible through Web Servers on the internet. Other sources for such programs are known to those of skill in the art. Alternatively, Vector NTI utilities are also used. There are also a number of algorithms known in the art that can be used to measure nucleotide sequence identity, including those contained in the programs described above. As another example, polynucleotide sequences can be compared using Fasta™, a program in GCG Version 6.1. Fasta™ provides alignments and percent sequence identity of the regions of the best overlap between the query and search sequences. For instance, percent sequence identity between nucleic acid sequences can be determined using Fasta™ with its default parameters (a word size of 6 and the NOP AM factor for the scoring matrix) as provided in GCG Version 6.1, herein incorporated by reference.

In some aspects, the modified hSMN1 coding sequence is a codon optimized sequence, optimized for expression in the subject species. As used herein, the “subject” is a mammal, e.g., a human, mouse, rat, guinea pig, dog, cat, horse, cow, pig, or non-human primate, such as a monkey, chimpanzee, baboon or gorilla. In some aspects, the subject is a human. Accordingly, in some aspects an SMN1 coding sequence is codon optimized for expression in a human.

Codon-optimized coding regions can be designed by various different methods. This optimization may be performed using methods which are available online (e.g., GeneArt), published methods, or a company which provides codon optimizing services, e.g., DNA2.0 (Menlo Park, Calif.). One codon optimizing method is described, e.g., in US International Patent Publication No. WO 2015/012924, which is incorporated by reference herein in its entirety. See also, e.g., US Patent Publication No. 2014/0032186 and US Patent Publication No. 2006/0136184.

In some aspects, the entire length of the open reading frame (ORF) is modified. However, in some aspects, only a fragment of the ORF is altered. By using one of these methods, one can apply the frequencies to any given polypeptide sequence, and produce a nucleic acid fragment of a codon-optimized coding region which encodes the polypeptide. Accordingly, in some aspects a codon optimized SMN1 coding sequence is used (e.g., a codon optimized hSMN1 ORF). In some aspects, one or more portions of the SMN1 coding sequence (e.g., up to the entire ORF) are codon optimized for expression in humans.

A number of options are available for performing the actual changes to the codons or for synthesizing the codon-optimized coding regions designed as described herein. Such modifications or synthesis can be performed using standard and routine molecular biological manipulations well known to those of ordinary skill in the art. In one approach, a series of complementary oligonucleotide pairs of 80-90 nucleotides each in length and spanning the length of the desired sequence are synthesized by standard methods. These oligonucleotide pairs are synthesized such that upon annealing, they form double stranded fragments of 80-90 base pairs, containing cohesive ends, e.g., each oligonucleotide in the pair is synthesized to extend 3, 4, 5, 6, 7, 8, 9, 10, or more bases beyond the region that is complementary to the other oligonucleotide in the pair. The single-stranded ends of each pair of oligonucleotides are designed to anneal with the single-stranded end of another pair of oligonucleotides. The oligonucleotide pairs are allowed to anneal, and approximately five to six of these double-stranded fragments are then allowed to anneal together via the cohesive single stranded ends, and then they ligated together and cloned into a standard bacterial cloning vector, for example, a TOPO® vector available from Invitrogen Corporation, Carlsbad, Calif. The construct is then sequenced by standard methods. Several of these constructs consisting of 5 to 6 fragments of 80 to 90 base pair fragments ligated together, i.e., fragments of about 500 base pairs, are prepared, such that the entire desired sequence is represented in a series of plasmid constructs. The inserts of these plasmids are then cut with appropriate restriction enzymes and ligated together to form the final construct. The final construct is then cloned into a standard bacterial cloning vector, and sequenced. Additional or alternative methods also could be used (including for example commercially available gene synthesis services).

In some aspects, SMN1 cDNA sequences can be generated in vitro and synthetically, using techniques known in the art. For example, the PCR-based accurate synthesis (PAS) of long DNA sequence method may be utilized, as described by Xiong et al, PCR-based accurate synthesis of long DNA sequences, Nature Protocols 1, 791-797 (2006). A method combining the dual asymmetrical PCR and overlap extension PCR methods is described by Young and Dong, Two-step total gene synthesis method, Nucleic Acids Res. 2004; 32(7): e59. See also, Gordeeva et al, J Microbiol Methods. Improved PCR-based gene synthesis method and its application to the Citrobacter freundii phytase gene codon modification. 2010 May; 81(2): 147-52. Epub 2010 Mar. 10; see, also, the following patents on oligonucleotide synthesis and gene synthesis, Gene Seq. 2012 April; 6(1): 10-21; U.S. Pat. Nos. 8,008,005; and 7,985,565. Each of these documents is incorporated herein by reference. In addition, kits and protocols for generating DNA via PCR are available commercially. These include the use of polymerases including, without limitation, Taq polymerase; OneTaq® (New England Biolabs); Q5® High-Fidelity DNA Polymerase (New England Biolabs); and GoTaq® G2 Polymerase (Promega). DNA may also be generated from cells transfected with plasmids containing the hSMN sequences described herein. Kits and protocols are known and commercially available and include, without limitation, QIAGEN plasmid kits; Chargeswitch® Pro Filter Plasmid Kits (Invitrogen); and GenElute™ Plasmid Kits (Sigma Aldrich). Other techniques useful herein include sequence-specific isothermal amplification methods that eliminate the need for thermocycling. Instead of heat, these methods typically employ a strand-displacing DNA polymerase, like Bst DNA Polymerase, Large Fragment (New England Biolabs), to separate duplex DNA. DNA may also be generated from RNA molecules through amplification via the use of Reverse Transcriptases (RT), which are RNA-dependent DNA Polymerases. RTs polymerize a strand of DNA that is complimentary to the original RNA template and is referred to as cDNA. This cDNA can then be further amplified through PCR or isothermal methods as outlined above. Custom DNA can also be generated commercially from companies including, without limitation, GenScript; GENEWIZ®; GeneArt® (Life Technologies); and Integrated DNA Technologies.

By “functional SMN1”, is meant a gene which encodes the native SMN protein or another SMN protein which provides at least about 50%, at least about 75%, at least about 80%, at least about 90%, or about the same, or greater than 100% of the biological activity level of the native survival of motor neuron protein, or a natural variant or polymorph thereof which is not associated with disease. Additionally, SMN1 homologue-SMN2 also encodes the SMN protein, but processes the functional protein less efficiently. Based on the copy number of SMN2, subjects lacking a functional hSMN1 gene demonstrate SMA to varying degrees. Thus, for some subjects, it may be desirable for the SMN protein to may provide less than 100% of the biological activity of the native SMN protein.

In some aspects, such a functional SMN has a sequence which has about 95% or greater identity to the native protein, or about 97% identity or greater, or about 99% at the amino acid level. Such a functional SMN protein may also encompass natural polymorphs. Identity may be determined by preparing an alignment of the sequences and through the use of a variety of algorithms and/or computer programs known in the art or commercially available (e.g., BLAST, ExPASy; ClustalO; FASTA; using, e.g., Needleman-Wunsch algorithm, Smith-Waterman algorithm).

Percent identity may be readily determined for amino acid sequences over the full-length of a protein, polypeptide, about 32 amino acids, about 330 amino acids, or a peptide fragment thereof or the corresponding nucleic acid sequence coding sequences. A suitable amino acid fragment may be at least about 8 amino acids in length, and may be up to about 700 amino acids. Generally, when referring to “identity”, “homology”, or “similarity” between two different sequences, “identity”, “homology” or “similarity” is determined in reference to “aligned” sequences.

In some aspects, modified SMN1 (e.g., hSMN1) genes described herein are engineered into a suitable genetic element (e.g., vector) useful for generating viral vectors and/or for delivery to a host cell, e.g., naked DNA, phage, transposon, cosmid, episome, etc., which transfers the SMN1 sequences carried thereon. The selected vector may be delivered by any suitable method, including transfection, electroporation, liposome delivery, membrane fusion techniques, high velocity DNA-coated pellets, viral infection and protoplast fusion. Methods used to make such constructs are known to those of skill in nucleic acid manipulation and include genetic engineering, recombinant engineering, and synthetic techniques. See, e.g., Sambrook et al, Molecular Cloning: A Laboratory Manual, Cold Spring Harbor Press, Cold Spring Harbor, N.Y.

In some aspects, an expression cassette comprising an SMN1 (e.g., a hSMN1) nucleic acid sequence(s) is provided. As used herein, an “expression cassette” refers to a nucleic acid molecule which comprises the SMN1 sequence operably linked to a promoter, and may include other regulatory sequences. In some aspects, the expression cassette is packaged into the capsid of a viral vector (e.g., a viral particle). Typically, such an expression cassette for generating a viral vector contains an SMN1 (e.g., an hSMN1) sequence described herein flanked by packaging signals of the viral genome and other expression control sequences such as those described herein. For example, for an AAV viral vector, the packaging signals are the 5′ inverted terminal repeat (ITR) and the 3′ ITR. When packaged into the AAV capsid, the ITRs in conjunction with the expression cassette, are referred to herein as the “recombinant AAV (rAAV) genome” or “vector genome” within an rAAV particle or capsid.

The term “expression” is used herein in its broadest meaning and comprises the production of RNA or of RNA and protein. With respect to RNA, the term “expression” or “translation” relates in particular to the production of peptides or proteins. Expression may be transient or may be stable.

The term “translation” in the context of the present invention relates to a process at the ribosome, wherein an mRNA strand controls the assembly of an amino acid sequence to generate a protein or a peptide.

### Promoters and Regulatory Elements:

In some aspects, an expression construct comprises one or more regions comprising a sequence that facilitates expression of the coding sequence of the SMN1 gene, e.g., expression control sequences operably linked to the coding sequence. Non-limiting examples of expression control sequences include promoters, insulators, silencers, response elements, introns, enhancers, initiation sites, termination signals, and poly(A) tails. Any combination of such control sequences is contemplated herein (e.g., a promoter and an enhancer).

In some aspects, an expression cassette contains a promoter sequence as part of the expression control sequences, e.g., located between the 5′ ITR sequence and the SMN1 coding sequence. The illustrative plasmid and vector described herein uses the ubiquitous chicken 3-actin promoter (CB) with CMV immediate early enhancer (CMV IE). Alternatively, other neuron-specific promoters may be used (see, e.g., the Lockery Lab neuron-specific promoters database, accessed at http://chinook.uoregon.edu/promoters.html). Such neuron-specific promoters include, without limitation, synapsin I (SYN), calcium/calmodulin-dependent protein kinase II, tubulin alpha I, neuron-specific enolase and platelet-derived growth factor beta chain promoters. See, Hioki et al, Gene Therapy, June 2007, 14(11):872-82, which is incorporated herein by reference. Other neuron-specific promoters include the 67 kDa glutamic acid decarboxylase (GAD67), homeobox Dlx5/6, glutamate receptor 1 (GluR1), preprotachykinin 1 (Tac1) promoter, neuron-specific enolase (NSE) and dopaminergic receptor 1 (Drd1a) promoters. See, e.g., Delzor et al, Human Gene Therapy Methods. August 2012, 23(4): 242-254. In another aspect, the promoter is a GUSb promoter http://www.jci.Org/articles/view/41615#B30.

Other promoters, such as constitutive promoters, regulatable promoters (see, e.g., WO 2011/126808 and WO 2013/04943), or a promoter responsive to physiologic cues may be used. Promoter(s) can be selected from different sources, e.g., human cytomegalovirus (CMV) immediate-early enhancer/promoter, the SV40 early enhancer/promoter, the JC polyomavirus promoter, myelin basic protein (MBP) or glial fibrillary acidic protein (GFAP) promoters, herpes simplex virus (HSV-1) latency associated promoter (LAP), rouse sarcoma virus (RSV) long terminal repeat (LTR) promoter, neuron-specific promoter (NSE), platelet derived growth factor (PDGF) promoter, hSYN, melanin-concentrating hormone (MCH) promoter, chicken beta-actin (CBA) promoter, and the matrix metalloprotein (MPP) promoter.

In addition to a promoter, an expression cassette and/or a vector may contain one or more other appropriate transcription initiation, termination, enhancer sequences, efficient RNA processing signals such as splicing and polyadenylation (poly A) signals; sequences that stabilize cytoplasmic mRNA for example WPRE; sequences that enhance translation efficiency (i.e., Kozak consensus sequence); sequences that enhance protein stability; and when desired, sequences that enhance secretion of the encoded product. Examples of suitable polyA sequences include, e.g., SV40, SV50, bovine growth hormone (bGH), human growth hormone, and synthetic poly As. An example of a suitable enhancer is the CMV enhancer. Other suitable enhancers include those that are appropriate for CNS indications. In some aspects, the expression cassette comprises one or more expression enhancers. In some aspects, the expression cassette contains two or more expression enhancers. These enhancers may be the same or may differ from one another. For example, an enhancer may include a CMV immediate early enhancer. This enhancer may be present in two copies which are located adjacent to one another. Alternatively, the dual copies of the enhancer may be separated by one or more sequences. In still another aspect, the expression cassette further contains an intron, e.g., the chicken beta-actin intron. Other suitable introns include those known in the art, e.g., such as are described in WO 2011/126808. In some aspects, an intron is incorporated upstream of the coding sequence to improve 5′ capping and stability of mRNA. Optionally, one or more other sequences may be selected to stabilize mRNA. An example of such a sequence is a modified WPRE sequence, which may be engineered upstream of the polyA sequence and downstream of the coding sequence (see, e.g., MA Zanta-Boussif, et al, Gene Therapy (2009) 16: 605-619).

In some aspects, these control sequences are “operably linked” to the SMN1 gene sequences. As used herein, the term “operably linked” refers to both expression control sequences that are contiguous with the gene of interest and expression control sequences that act in trans or at a distance to control the gene of interest.

### Recombinant Viral Vectors:

In some aspects, an adeno-associated viral vector that comprises an AAV capsid and at least one expression cassette is provided. In some aspects, the at least one expression cassette comprises nucleic acid sequences encoding SMN1 and expression control sequences that direct expression of the SMN1 sequences in a host cell. An rAAV vector gene can also comprises AAV ITR sequences. In some aspects, the ITRs are from an AAV serotype that is different from the serotype of the capsid proteins used to package the rAAV genome. In some aspects, the ITR sequences are from AAV2, or the deleted version thereof (AITR), which may be used for convenience and to accelerate regulatory approval. However, ITRs from other AAV sources may be selected. Where the source of the ITRs is from AAV2 and the AAV capsid is from another AAV source, the resulting vector may be termed pseudotyped. Typically, rAAV vector genomes comprise an AAV 5′ ITR, the SMN1 coding sequences and any regulatory sequences, and an AAV 3′ ITR. However, other configurations of these elements may be suitable. A shortened version of the 5′ ITR, termed AITR, has been described in which the D-sequence and terminal resolution site (trs) are deleted. In other aspects, the full-length AAV 5′ and 3′ ITRs are used.

The ITR sequences of a nucleic acid or nucleic acid vector described herein can be derived from any AAV serotype (e.g., 1, 2, 3, 4, 5, 6, 7, 8, 9, or 10) or can be derived from more than one serotype. In some aspects, ITR sequences and plasmids containing ITR sequences are known in the art and commercially available (see, e.g., products and services available from Vector Biolabs, Philadelphia, Pa.; Cellbiolabs, San Diego, Calif.; Agilent Technologies, Santa Clara, Calif.; and Addgene, Cambridge, Mass.; and Gene delivery to skeletal muscle results in sustained expression and systemic delivery of a therapeutic protein. Kessler P D, Podsakoff G M, Chen X, McQuiston S A, Colosi P C, Matelis L A, Kurtzman G J, Byrne B J. Proc Natl Acad Sci USA. 1996 Nov. 26; 93(24):14082-7; and Curtis A. Machida. Methods in Molecular Medicine™. Viral Vectors for Gene Therapy Methods and Protocols. 10.1385/1-59259-304-6:201 © Humana Press Inc. 2003. Chapter 10. Targeted Integration by Adeno-Associated Virus. Matthew D. Weitzman, Samuel M. Young Jr., Toni Cathomen and Richard Jude Samulski; U.S. Pat. Nos. 5,139,941 and 5,962,313, all of which are incorporated herein by reference).

In some aspects, rAAV nucleic acids or genomes can be single-stranded (ss). However, in some aspects, rAAV nucleic acids or genomes can be self-complementary (sc) AAV nucleic acid vectors. In some aspects, a recombinant AAV particle comprises a nucleic acid vector, such as a single-stranded (ss) or self-complementary (sc) AAV nucleic acid vector. In some aspects, the nucleic acid vector contains an SMN1 gene and one or more regions comprising inverted terminal repeat (ITR) sequences (e.g., wild-type ITR sequences or engineered ITR sequences) flanking the expression construct. In some aspects, the nucleic acid is encapsidated by a viral capsid.

Accordingly, in some aspects, a AAV particle comprises a viral capsid and a nucleic acid vector as described herein, which is encapsidated by the viral capsid. In some aspects, the viral capsid comprises 60 capsid protein subunits comprising VP1, VP2 and VP3. In some aspects, the VP1, VP2, and VP3 subunits are present in the capsid at a ratio of approximately 1:1:10, respectively.

In some aspects, a recombinant adeno-associated virus (rAAV) is an AAV DNase-resistant particle having an AAV protein capsid into which is packaged nucleic acid sequences for delivery to target cells. In some aspects, an AAV capsid is composed of 60 capsid (cap) protein subunits, VP1, VP2, and VP3, that are arranged in an icosahedral symmetry in a ratio of approximately 1:1:10 to 1:1:20, depending upon the selected AAV. The AAV capsid may be chosen from those known in the art, including variants thereof. In some aspects, the AAV capsid is chosen from those that effectively transduce neuronal cells. In some aspects, the AAV capsid is selected from AAV1, AAV2, AAV7, AAV 8, AAV9, AAVrh10, AAV5, AAVhu11, AAV8DJ, AAVhu32, AAVhu37, AAVpi2, AAVrh8, AAVhu48R3, AAVhu68 and variants thereof. See, WO2018160585A2, WO2018160582A1, Royo, et al, Brain Res, 2008 January, 1190: 15-22; Petrosyan et al, Gene Therapy, 2014 Dec., 21(12):991-1000; Holehonnur et al, BMC Neuroscience, 2014, 15:28; and Cearley et al, Mol Ther. 2008 October; 16(10): 1710-1718, each of which is incorporated herein by reference. Other AAV capsids useful herein include AAVrh39, AAVrh20, AAVrh25, AAV10, AAVbb1, and AAVbb2 and variants thereof. Other AAV serotypes may be selected as sources for capsids of AAV viral vectors (DNase resistant viral particles) including, e.g., AAV1, AAV2, AAV3, AAV4, AAV5, AAV6, AAV6.2, AAV7, AAV8, AAV9, AAVrh10, AAVrh64R1, AAVrh64R2, AAVrh8, and variants of any of the known or mentioned AAVs or AAVs yet to be discovered. See, e.g., US Published Patent Application No. 2007-0036760-A1; US Published Patent Application No. 2009-0197338-A1; EP 1310571. See also, WO 2003/042397 (AAV7 and other simian AAV), U.S. Pat. Nos. 7,790,449 and 7,282,199 (AAV8), WO 2005/033321 and U.S. Pat. No. 7,906,111 (AAV9), and WO 2006/110689, and WO 2003/042397 (rh10). Alternatively, a recombinant AAV based upon any of the recited AAVs, may be used as a source for the AAV capsid. These documents also describe other AAV which may be selected for generating AAV and are incorporated by reference. In some aspects, an AAV cap for use in the viral vector can be generated by mutagenesis (e.g., by insertions, deletions, or substitutions) of one of the aforementioned AAV Caps or its encoding nucleic acid. In some aspects, the AAV capsid is chimeric, comprising domains from two or three or four or more of the aforementioned AAV capsid proteins. In some aspects, the AAV capsid is a mosaic of Vp1, Vp2, and Vp3 monomers from two or three different AAVs or recombinant AAVs. In some aspects, an rAAV composition comprises more than one of the aforementioned Caps. As used herein, relating to AAV, the term variant means any AAV sequence which is derived from a known AAV sequence, including those sharing at least 70%, at least 75%, at least 80%, at least 85%, at least 90%, at least 95%, at least 97%, at least 99% or greater sequence identity over the amino acid or nucleic acid sequence. In another aspect, the AAV capsid includes variants which may include up to about 10% variation from any described or known AAV capsid sequence. That is, the AAV capsid shares about 90% identity to about 99.9% identity, about 95% to about 99% identity or about 97% to about 98% identity to an AAV capsid provided herein and/or known in the art. In some aspects, the AAV capsid shares at least 95% identity with an AAV capsid. When determining the percent identity of an AAV capsid, the comparison may be made over any of the variable proteins (e.g., vp1, vp2, or vp3). In some aspects, the AAV capsid shares at least 95% identity with the AAV8 vp3.

In some aspects, a self-complementary AAV is provided. The abbreviation “sc” in this context refers to self-complementary. “Self-complementary AAV” refers a construct in which a coding region carried by a recombinant AAV nucleic acid sequence has been designed to form an intra-molecular double-stranded DNA template. Upon infection, rather than waiting for cell mediated synthesis of the second strand, the two complementary halves of scAAV will associate to form one double stranded DNA (dsDNA) unit that is ready for immediate replication and transcription. See, e.g., D M McCarty et al, “Self-complementary recombinant adeno-associated virus (scAAV) vectors promote efficient transduction independently of DNA synthesis”, Gene Therapy, (August 2001), Vol 8, Number 16, Pages 1248-1254. Self-complementary AAVs are described in, e.g., U.S. Pat. Nos. 6,596,535; 7,125,717; and 7,456,683, each of which is incorporated herein by reference in its entirety.

Methods for generating and isolating AAV viral vectors suitable for delivery to a subject are known in the art. See, e.g., US Published Patent Application No. 2007/0036760 (Feb. 15, 2007), U.S. Pat. Nos. 7,790,449; 7,282,199; WO 2003/042397; WO 2005/033321, WO 2006/110689; and U.S. Pat. No. 7,588,772 B2. In one system, a producer cell line is transiently transfected with a construct that encodes the transgene flanked by ITRs and a construct(s) that encodes rep and cap. In a second system, a packaging cell line that stably supplies rep and cap is transiently transfected with a construct encoding the transgene flanked by ITRs. In each of these systems, AAV virions are produced in response to infection with helper adenovirus or herpesvirus, requiring the separation of the rAAVs from contaminating virus. Systems also have been developed that do not require infection with helper virus to recover the AAV—the required helper functions (e.g., adenovirus E1, E2a, VA, and E4 or herpesvirus UL5, UL8, UL52, and UL29, and herpesvirus polymerase) are also supplied, in trans, by the system. In these systems, the helper functions can be supplied by transient transfection of the cells with constructs that encode the required helper functions, or the cells can be engineered to stably contain genes encoding the helper functions, the expression of which can be controlled at the transcriptional or posttranscriptional level. In yet another system, the transgene flanked by ITRs and rep/cap genes are introduced into insect cells by infection with baculovirus-based vectors. For reviews on these production systems, see generally, e.g., Zhang et al, 2009, “Adenovirus-adeno-associated virus hybrid for large-scale recombinant adeno-associated virus production,” Human Gene Therapy 20:922-929, the contents of each of which is incorporated herein by reference in its entirety. Methods of making and using these and other AAV production systems are also described in the following U.S. patents, the contents of each of which is incorporated herein by reference in its entirety: U.S. Pat. Nos. 5,139,941; 5,741,683; 6,057,152; 6,204,059; 6,268,213; 6,491,907; 6,660,514; 6,951,753; 7,094,604; 7,172,893; 7,201,898; 7,229,823; and 7,439,065.

Optionally, the SMN1 genes described herein may be used to generate viral vectors other than rAAV, and that also can be used in combination therapy with SMN2 ASOs. Such other viral vectors may include any virus suitable for gene therapy may be used, including but not limited to adenovirus; herpes virus; lentivirus; retrovirus etc. Suitably, where one of these other vectors is generated, it is produced as a replication-defective viral vector.

A “replication-defective virus” or “viral vector” refers to a synthetic or artificial viral particle in which an expression cassette containing a gene of interest is packaged in a viral capsid or envelope, where any viral genomic sequences also packaged within the viral capsid or envelope are replication-deficient; i.e., they cannot generate progeny virions but retain the ability to infect target cells. In some aspects, the genome of the viral vector does not include genes encoding the enzymes required to replicate (the genome can be engineered to be “gutless”-containing only the transgene of interest flanked by the signals required for amplification and packaging of the artificial genome), but these genes may be supplied during production. Therefore, it is deemed safe for use in gene therapy since replication and infection by progeny virions cannot occur except in the presence of the viral enzyme required for replication. Such replication-defective viruses may be adeno-associated viruses (AAV), adenoviruses, lentiviruses (integrating or non-integrating), or another suitable virus source.

Host cells that comprise at least one of the disclosed AAV particles, expression constructs, or nucleic acid vectors also are provided. Such host cells include mammalian host cells, for example human host cells, and may be either isolated, in cell or tissue culture. In the case of genetically modified animal models (e.g., a mouse), the transformed host cells may be comprised within the body of a non-human animal itself.

Oligomeric Compounds that Increase Full-Length SMN2 mRNA Production

In some aspects, a combined therapy for treating SMA includes administering (e.g., concurrently or sequentially) ASOs complementary to a pre-mRNA encoding SMN2 (also referred to as SMN2 ASOs in this application) in addition to other therapies described herein (e.g., a recombinant SMN1 gene and/or a small molecule that increases SMN function). In some aspects, the ASO increases full-length SMN2 mRNA. In some aspects, the ASO alters splicing of SMN2 pre-mRNA. In some aspects, the ASO promotes exon 7 inclusion in SMN2 mRNA. Some sequences and regions useful for altering splicing of SMN2 may be found in PCT/US06/024469 (published as WO/2007/002390) and WO2018014041A2, which are hereby incorporated by reference in their entirety for any purpose.

In some aspects, SMN2 ASOs effectively modulate splicing of SMN2, resulting in an increase in exon 7 inclusion in SMN2 mRNA and ultimately in SMN2 protein that includes the amino acids corresponding to exon 7. Such alternate SMN2 protein is 100% identical to wild-type SMN protein.

ASOs that effectively modulate expression of SMN2 mRNA to produce functional SMN protein are considered active ASOs. Modulation of expression of SMN2 can be measured in a bodily fluid, which may or may not contain cells; tissue; or organ of the animal. Methods of obtaining samples for analysis, such as body fluids (e.g., sputum, serum, CSF), tissues (e.g., biopsy), or organs, and methods of preparation of the samples to allow for analysis are well known to those skilled in the art. The effects of treatment can be assessed by measuring biomarkers associated with the target gene expression in one or more biological fluids, tissues or organs, collected from an animal contacted with one or more compositions described in this application.

In some aspects, an increase in full-length SMN2 mRNA means that the intracellular level of full-length SMN2 mRNA is higher than a reference level, such as the level of full-length SMN2 mRNA in a control (for example in a subject that is not being administered a SMN2 ASO). An increase in intracellular full-length SMN2 mRNA can be measured as an increase in the level of full-length protein and/or mRNA produced from the SMN2 gene. In some aspects, an increase in full-length SMN2 mRNA can be determined by examination of the outward properties of the cell or organism (e.g., as described below in the examples), or by assay techniques such as RNA solution hybridization, nuclease protection, Northern hybridization, reverse transcription, gene expression monitoring with a microarray, antibody binding, enzyme linked immunosorbent assay (ELISA), nucleic acid sequencing, Western blotting, radioimmunoassay (RIA), other immunoassays, fluorescence activated cell analysis (FACS), or any other technique or combination of techniques that can detect the presence of full-length SMN2 mRNA or protein (e.g., in a subject or a sample obtained from a subject).

In some aspects, by comparing the level of full-length SMN2 mRNA in a sample obtained from a subject receiving a SMN2 ASO treatment to a level of full-length SMN2 mRNA in a subject not treated with the SMN2 ASO, the extent to which the SMN2 ASO increased full-length SMN2 mRNA can be determined. In some aspects, the reference level of full-length SMN2 mRNA is obtained from the same subject prior to receiving SMN2 ASO. In some aspects, the reference level of full-length SMN2 mRNA is a range determined by a population of subjects not receiving SMN2 ASO.

In some aspects, an increased level of full-length SMN2 mRNA is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference value.

In some aspects, by comparing the ratio of full-length SMN2 mRNA to a shorter SMN2 mRNA (e.g., SMN2 mRNA without exon 7) with a reference ratio in a subject receiving SMN2 ASO administration, it can be determined whether the SMN2 ASO resulted in an increase of full-length SMN2 mRNA. In some aspects, the reference ratio is the ratio of the full length SMN2 mRNA to a short SMN2 mRNA (e.g., SMN2 mRNA without exon 7) prior to SMN2 ASO administration. In some aspects, the ratio of the full length SMN2 mRNA to a short SMN2 mRNA (e.g., SMN2 mRNA without exon 7) in a subject receiving SMN2 ASO is, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference ratio.

In some aspects, the increase of full-length SMN2 mRNA in a subject can be indicated by the increase of full-length SMN protein as compared to a reference level. In some aspects, the reference level of full-length SMN protein is the full-length SMN protein level obtained from a subject having or at risk of having SMA prior to treatment. In some aspects, exon 7-containing SMN protein production is increased in a subject receiving SMN2 ASO administration with an enhancement of exon 7-containing SMN protein levels of at least about, for example, greater than 1 fold, 1.5-5 fold, 5-10 fold, 10-50 fold, 50-100 fold, about 1.1-, 1.2-, 1.5-, 2-, 3-, 4-, 5-, 6-, 7-, 8-, 9-, 10-, 15-, 20-, 30-, 40-, 50-, 60-, 70-, 80-, 90-, 100-fold or more higher than a reference value. Methods whereby bodily fluids, organs or tissues are contacted with an effective amount of one or more compositions described in this application are also contemplated. Bodily fluids, organs or tissues can be contacted with one or more compositions resulting in expression of SMN1 and modulation of SMN2 expression in the cells of bodily fluids, organs or tissues. An effective amount of a composition can be determined by monitoring the effect on functional SMN protein expression of recombinant SMN1 genes and SMN2 ASOs that are administered to a subject or contacted to a cell.

1. Antisense Oligonucleotides (ASOs)

In some aspects, an ASO comprising a sequence complementary to a nucleic acid encoding human SMN2 is provided for use in treating (e.g., with a recombinant SMN1 gene and/or a small molecule that increases SMN function) a disease or condition associated with survival motor neuron protein (SMN), such as spinal muscular atrophy (SMA). In some aspects, an ASO comprising a sequence complementary to a nucleic acid encoding human SMN2 is provided for use in treating (e.g., with a recombinant SMN1 gene and/or a small molecule that increases SMN function) a disease or condition associated with survival motor neuron protein (SMN) by administering the ASO directly into the central nervous system (CNS) or CSF.

As used herein, the term “oligomeric compound” refers to a compound comprising an oligonucleotide. In some aspects, an oligomeric compound consists of an oligonucleotide. As used herein, the term “oligonucleotide” refers to a compound comprising a phosphate linking group, a heterocyclic base moiety and a sugar moiety. In some aspects, an oligomeric compound further comprises one or more conjugate and/or terminal groups. In some aspects, oligomeric compounds are antisense oligonucleotides (ASO). As used herein, the terms “antisense oligonucleotide” or “ASO” refer to an oligomeric compound, at least a portion of which is at least partially complementary to a target nucleic acid to which it hybridizes, wherein such hybridization results at least one antisense activity.

In some instances, an antisense oligonucleotide (ASO) increases full-length SMN protein in the subject. In some instances, the ASO increases the full-length SMN2 mRNA in a subject. In some aspects, an ASO that increases the full-length SMN2 mRNA is an antisense oligonucleotide that is complementary to a nucleic acid encoding SMN2. In some aspects, the ASO increases full-length SMN2 mRNA by altering the splicing pattern of SMN2 pre-mRNA. In some aspects the ASO promotes exon skipping during splicing of SMN2 pre-mRNA. In some aspects, the ASO promotes the inclusion of exon 7 in the SMN2 mRNA. In some aspects, the ASO is designed to target, intron 6, intron 7, or the boundary between exon 7 and an adjacent intron of SMN2 pre-mRNA to promote the inclusion of exon 7 in the SMN2 mRNA. In some aspects, the ASO comprises a nucleobase sequence complementary to intron 6 of SMN2 pre-mRNA. In some aspects, the ASO comprises a nucleobase sequence complementary to exon 6 of SMN2 pre-mRNA. In some aspects, the ASO comprises a nucleobase sequence complementary to intron 7 of SMN2 pre-mRNA. In some aspects, the ASO targeting intron 7 of SMN2 pre-mRNA comprises a nucleotide sequence of SEQ ID NO: 1. In some aspects, the ASO targeting intron 7 of SMN2 pre-mRNA is nusinersen. In some aspects, one or more of the ASOs described herein can be administered to a subject for increased level of full-length SMN protein and/or full-length SMN2 mRNA. Non-limiting examples of sequences and regions useful for altering splicing of SMN2 may be found in PCT/USO_{6/024469}, which is hereby incorporated by reference in its entirety for any purpose. In some aspects, an antisense oligonucleotide has a nucleobase sequence that is complementary to intron 7 of SMN2. Non-limiting examples of such nucleobase sequences are exemplified in the table below.

| Sequence | Length | SEQ ID NO |
|---|---|---|
| TGCTGGCAGACTTAC | 15 | 2 |
| CATAATGCTGGCAGA | 15 | 3 |
| TCATAATGCTGGCAG | 15 | 4 |
| TTCATAATGCTGGCA | 15 | 5 |
| TTTCATAATGCTGGC | 15 | 6 |
| ATTCACTTTCATAATGCTGG | 20 | 7 |
| TCACTTTCATAATGCTGG | 18 | 1 |
| CTTTCATAATGCTGG | 15 | 8 |
| TCATAATGCTGG | 12 | 9 |
| ACTTTCATAATGCTG | 15 | 10 |
| TTCATAATGCTG | 12 | 11 |
| CACTTTCATAATGCT | 15 | 12 |
| TTTCATAATGCT | 12 | 13 |
| TCACTTTCATAATGC | 15 | 14 |
| CTTTCATAATGC | 12 | 15 |
| TTCACTTTCATAATG | 15 | 16 |
| ACTTTCATAATG | 12 | 17 |
| ATTCACTTTCATAAT | 15 | 18 |
| CACTTTCATAAT | 12 | 19 |
| GATTCACTTTCATAA | 15 | 20 |
| TCACTTTCATAA | 12 | 21 |
| TTCACTTTCATA | 12 | 22 |
| ATTCACTTTCAT | 12 | 23 |
| AGTAAGATTCACTTT | 15 | 24 |

In some aspects, the ASO targets intron 7 of SMN2 pre-mRNA. In some aspects, an ASO comprises a nucleobase sequence comprising at least 10 nucleobases of the sequence: TCACTTTCATAATGCTGG (SEQ ID NO: 1). In some aspects, an ASO has a nucleobase sequence comprising at least 11 nucleobases of SEQ ID NO: 1. In some aspects, an ASO has a nucleobase sequence comprising at least 12 nucleobases of SEQ ID NO: 1. In some aspects, an ASO has a nucleobase sequence comprising at least 13 nucleobases of SEQ ID NO: 1. In some aspects, an ASO has a nucleobase sequence comprising at least 14 nucleobases of SEQ ID NO: 1. In some aspects, an ASO has a nucleobase sequence comprising at least 15 nucleobases of SEQ ID NO: 1. In some aspects, an ASO has a nucleobase sequence comprising at least 16 nucleobases of SEQ ID NO: 1. In some aspects, an ASO has a nucleobase sequence comprising at least 17 nucleobases of SEQ ID NO: 1. In some aspects, an ASO has a nucleobase sequence comprising the nucleobases of SEQ ID NO: 1. In some aspects, an ASO has a nucleobase sequence consisting of the nucleobases of SEQ ID NO: 1. In some aspects, an ASO consists of 10-18 linked nucleosides and has a nucleobase sequence 100% identical to an equal-length portion of the sequence: TCACTTTCATAATGCTGG (SEQ ID NO: 1).

In some aspects, SMN2 ASOs are complementary to a nucleic acid molecule encoding the SMN2 protein. In some aspects, the ASOs are complementary to intron 6, exon 7 (or the boundary of exon 7 and an adjacent intron), or intron 7 of a nucleic acid molecule encoding SMN2 protein. In some aspects, the ASO targets intron 7 of SMN2 pre-mRNA. In some aspects, a SMN2 ASO targeting intron 7 of SMN2 pre-mRNA is nusinersen. An exemplary nucleotide sequence for nusinersen is 5′-UCACUUUCAUAAUGCUGG-3′ (SEQ ID NO: 26). The active substance, nusinersen (also referred to as ISIS 396443), is a uniformly modified 2′-O-(2-methoxyethyl) phosphorothioate antisense oligonucleotide consisting of 18 nucleotide residues having the sequence 5′-^{Me}U^{Me}CA^{Me}C^{Me}U^{Me}U^{Me}U^{Me}CA^{Me}UAA^{Me}UG^{Me}C^{Me}UGG-3′ (SEQ ID NO: 25). In some aspects, the SMN2 ASO comprises nucleobase sequence comprising the nucleobases of SEQ ID NO: 25 or 26.

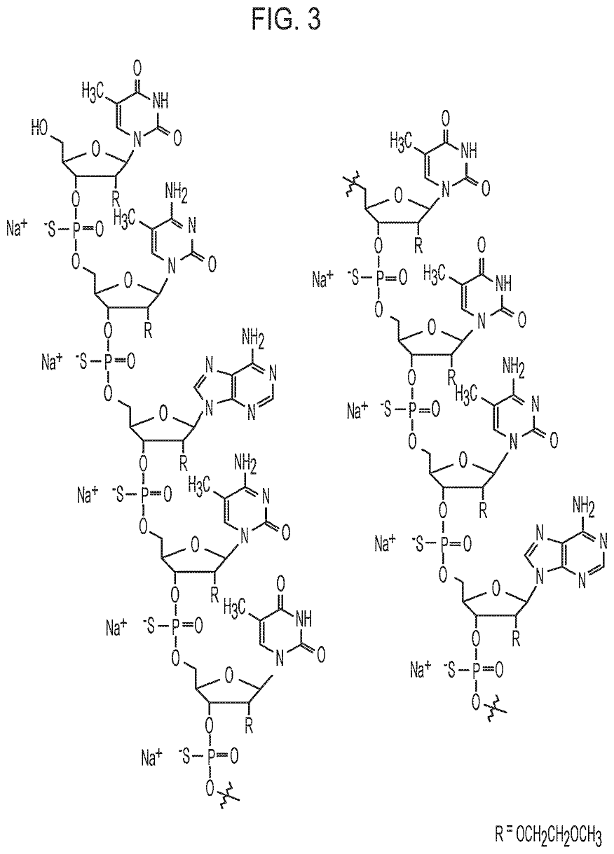

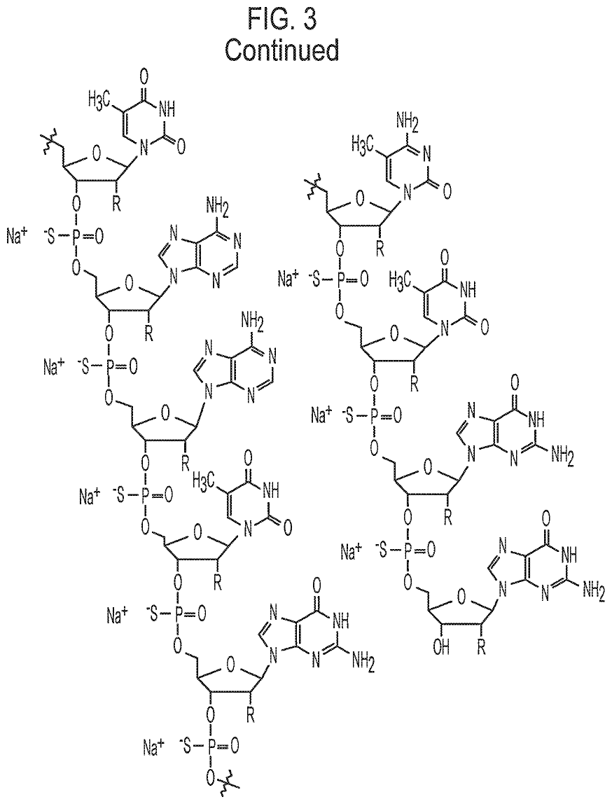

The chemical name of nusinersen sodium is 2′-O-(2-methoxyethyl)-5-methyl-P-thiouridylyl-(3′-O→5′-O)-2′-O-(2-methoxyethyl)-5-methyl-P-thiocytidylyl-(3′-O→5′-O)-2′-O-(2-methoxyethyl)-P-thioadenylyl-(3′-O→5′-O)-2′-O-(2-methoxyethyl)-5-methyl-P-thiocytidylyl-(3′-O→5′-O)-2′-O-(2-methoxyethyl)-5-methyl-P-thiouridylyl-(3′-O→5′-O)-2′-O-(2-methoxyethyl)-5-methyl-P-thiouridylyl-(3′-O→5′-O)-2′-O-(2-methoxyethyl)-5-methyl-P-thiouridylyl-(3′-O→5′-O)-2′-O-(2-methoxyethyl)-5-methyl-P-thiocytidylyl-(3′-O→5′-0)-2′-O-(2-methoxyethyl)-P-thioadenylyl-(3′-O→5′-O)-2′-O-(2-methoxyethyl)-5-methyl-P-thiouridylyl-(3′-O→5′-O)-2′-O-(2-methoxyethyl)-P-thioadenylyl-(3′-O→5′-O)-2′-O-(2-methoxyethyl)-P-thioadenylyl-(3′-O→5′-O)-2′-O-(2-methoxyethyl)-5-methyl-P-thiouridylyl-(3′-O→5′-O)-2′-O-(2-methoxyethyl)-P-thioguanylyl-(3′-O→5′-O)-2′-O-(2-methoxyethyl)-5-methyl-P-thiocytidylyl-(3′-O→5′-O)-2′-O-(2-methoxyethyl)-5-methyl-P-thiouridylyl-(3′-O→5′-O)-2′-O-(2-methoxyethyl)-P-thioguanylyl-(3′-O→5′-O)-2′-O-(2-methoxyethyl)guanosine corresponding to the molecular formula C234H323N61O128P17S17Na17 and has a relative molecular mass 7501.0 g/mol and the structure shown in FIG. 3.

Antisense is an effective means for modulating the expression of one or more specific gene products and is uniquely useful in a number of therapeutic, diagnostic, and research applications. Provided herein are antisense compounds useful for modulating gene expression via antisense mechanisms of action, including antisense mechanisms based on target occupancy. In one aspect, the antisense compounds provided herein modulate splicing of a target gene. Such modulation includes promoting or inhibiting exon inclusion. Further provided herein are antisense compounds targeted to cis splicing regulatory elements present in pre-mRNA molecules, including exonic splicing enhancers, exonic splicing silencers, intronic splicing enhancers and intronic splicing silencers. Disruption of cis splicing regulatory elements is thought to alter splice site selection, which may lead to an alteration in the composition of splice products.

Processing of eukaryotic pre-mRNAS is a complex process that requires a multitude of signals and protein factors to achieve appropriate mRNA splicing. Exon definition by the spliceosome requires more than the canonical splicing signals which define intron-exon boundaries. One such additional signal is provided by cis-acting regulatory enhancer and silencer sequences. Exonic splicing enhancers (ESE), exonic splicing silencers (ESS), intronic splicing enhancers (ISE) and intron splicing silencers (ISS) have been identified which either repress or enhance usage of splice donor sites or splice acceptor sites, depending on their site and mode of action (Yeo et al. 2004, Proc. Natl. Acad. Sci. U.S.A. 101(44): 15700-15705). Binding of specific proteins (trans factors) to these regulatory sequences directs the splicing process, either promoting or inhibiting usage of particular splice sites and thus modulating the ratio of splicing products (Scamborova et al. 2004, Mol. Cell. Biol. 24(5):1855-1869: Hovhannisyan and Carstens, 2005, Mol. Cell. Biol. 25(1):250-263; Minovitsky et al. 2005, Nucleic Acids Res. 33(2):714-724).

In some aspects, antisense oligonucleotides comprise one or more modifications compared to oligonucleotides of naturally occurring oligomers, such as DNA or RNA. Such modified antisense oligonucleotides may possess one or more desirable properties. In some aspects, modifications alter the antisense activity of the antisense oligonucleotide, for example by increasing affinity of the antisense oligonucleotide for its target nucleic acid, increasing its resistance to one or more nucleases, and/or altering the pharmacokinetics or tissue distribution of the oligonucleotide. In some aspects, modified antisense oligonucleotides comprise one or more modified nucleosides and/or one or more modified nucleoside linkages and/or one or more conjugate groups.

a. Modified Nucleosides

In some aspects, antisense oligonucleotides comprise one or more modified nucleosides. Such modified nucleosides may include a modified sugar and/or a modified nucleobase. In some aspects, incorporation of such modified nucleosides in an oligonucleotide results in increased affinity for a target nucleic acid and/or increased stability, including but not limited to, increased resistance to nuclease degradation, and or improved toxicity and/or uptake properties of the modified oligonucleotide.

i. Nucleobases

The naturally occurring base portion of nucleosides are heterocyclic bases, typically purines and pyrimidines. In addition to “unmodified” or “natural” nucleobases such as the purine nucleobases adenine (A) and guanine (G), and the pyrimidine nucleobases thymine (T), cytosine (C) and uracil (U), many modified nucleobases or nucleobase mimetics known to those skilled in the art are amenable to incorporation into the compounds described herein. In some aspects, a modified nucleobase is a nucleobase that is fairly similar in structure to the parent nucleobase, such as for example a 7-deaza purine, a 5-methyl cytosine, or a G-clamp. In some aspects, nucleobase mimetics include more complicated structures, such as for example a tricyclic phenoxazine nucleobase mimetic. Methods for preparing modified nucleobases are well known to those skilled in the art.

ii. Modified Sugars and Sugar Surrogates

Antisense oligonucleotides of the present application can optionally contain one or more nucleosides wherein the sugar moiety is modified, compared to a natural sugar. Oligonucleotides comprising sugar modified nucleosides may have enhanced nuclease stability, increased binding affinity or some other beneficial biological property. Such modifications include without limitation, addition of substituent groups, bridging of non-geminal ring atoms to form a bicyclic nucleic acid (BNA), replacement of the ribosyl ring oxygen atom with S, N(R), or C(R₁)(R)₂ (R═H, C₁-C₁₂ alkyl or a protecting group) and combinations of these such as for example a 2′-F-5′-methyl substituted nucleoside (see PCT International Application WO 2008/101157 Published on Aug. 21, 2008 for other disclosed 5′,2′-bis substituted nucleosides) or replacement of the ribosyl ring oxygen atom with S with further substitution at the 2′-position (see published U.S. Patent Application US20050130923, published on Jun. 16, 2005) or alternatively 5′-substitution of a BNA (see PCT International Application WO 2007/134181 Published on Nov. 22, 2007 wherein LNA is substituted with for example a 5′-methyl or a 5′-vinyl group).

Examples of nucleosides having modified sugar moieties include without limitation nucleosides comprising 5′-vinyl, 5′-methyl (R or S), 4′-S, 2′-F, 2′-OCH and 2′-O(CH₂)₂OCH₃ substituent groups. The substituent at the 2′ position can also be selected from allyl, amino, azido, thio, O-allyl, O—C₁-C₁₀ alkyl, OCF₃, O(CH₂)SCH₃, O(CH₂)₂—O—N(Rₘ)(Rₙ), and O—CH₂—C(═O) N(Rₘ)(Rₙ), where each Rₘ, and Rₙ is, independently, H or substituted or unsubstituted C₁-C₁₀ alkyl.

Examples of bicyclic nucleic acids (BNAs) include without limitation nucleosides comprising a bridge between the 4′ and the 2′ ribosyl ring atoms. In some aspects, antisense compounds provided herein include one or more BNA nucleosides wherein the bridge comprises one of the formulas: 4′-beta-D-(CH₂)—O-2′ (beta-D-LNA); 4′-(CH₂)—S-2: 4′-alpha-L-(CH₂)—O-2′ (alpha-L-LNA); 4′-(CH₂)₂—O-2′ (ENA); 4′-C(CH₃)₂—O-2′ (see PCT/US2008/068922); 4′-CH(CH₃) O-2′ and 4′-C—H(CH₂OCH₃) O-2′ (see U.S. Pat. No. 7,399,845, issued on Jul. 15, 2008): 4′-CH₂—N(OCH₃)-2′ (see PCT/US2008/064591); 4′-CH₂—O—N(CH₃)-2′ (see published U.S. Patent Application US2004-0171570, published Sep. 2, 2004): 4′-CH₂—N(R)—O-2′ (see U.S. Pat. No. 7,427,672, issued on Sep. 23, 2008): 4′-CH₂—C(CH₃)-2′ and 4′-CH₂—C(═CH₂)-2′ (see PCT/US2008/066154); and wherein R is, independently, H, C₁-C₁₂ alkyl, or a protecting group.

In some aspects, modified nucleosides comprising modified sugar moieties are not bicyclic sugar moieties. In some aspects, the sugar ring of a nucleoside may be modified at any position. Examples of useful sugar modifications include, but are not limited to, compounds comprising a sugar substituent group selected from: OH, F, O-alkyl, S-alkyl, N-alkyl, or O-alkyl-O-alkyl, wherein the alkyl, alkenyl and alkynyl may be substituted or unsubstituted C₁ to C₁₀ alkyl or C₂ to C₁₀ alkenyl and alkynyl. In some aspects, such substituents are at the 2′ position of the sugar.

In some aspects, modified nucleosides comprise a substituent at the 2′ position of the sugar. In some aspects, such substituents are selected from among: a halide (including, but not limited to F), allyl, amino, azido, thio. O-allyl, O—C₁-C₁₀ alkyl, —OCF₃, O—(CH₂)₂—O—CH₃, 2′-O(CH₂)₂SCH₃, O—(CH₂)₂—O—N(Rₘ)(Rₙ), or O—CH₂—C(═O)—N(Rₘ)(Rₙ), where each Rₘ, and Rₙ is, independently, H or substituted or unsubstituted C₁-C₁₀ alkyl.

In some aspects, modified nucleosides suitable for use in the present invention are: 2-methoxyethoxy, 2′-Omethyl (2′-O CH₃), 2′-fluoro (2′-F).

In some aspects, modified nucleosides having a substituent group at the 2′-position selected from: O[(CH₂)ₙO]ₘCH₃, O(CH₂), NH₂, O(CH₂)₂CH₃, O(CH₂), ONH₂, OCH₂C(═O)N(H)CH₃, and O(CH₂)ₙON[(CH₂)ₙCH₃]₂, where n and m are from 1 to about 10. Other 2′-sugar substituent groups include: C₁ to C₁₀ alkyl, substituted alkyl, alkenyl, alkynyl, alkaryl, aralkyl, O-alkaryl or O-aralkyl, SH, SCH, OCN, Cl, Br, CN, CF₃, OCF₃, SOCH₃, SO₂CH₃, ONO₂, NO₂, N₃, NH₂, heterocycloalkyl, heterocycloalkaryl, aminoalkylamino, polyalkylamino, substituted silyl, an RNA cleaving group, a reporter group, an intercalator, a group for improving pharmacokinetic properties, or a group for improving the pharmacodynamic properties of an oligomeric compound, and other substituents having similar properties.

In some aspects, modified nucleosides comprise a 2′-MOE side chain (Baker et al., J. Biol. Chem., 1997, 272, 11944-12000). Such 2′-MOE substitution have been described as having improved binding affinity compared to unmodified nucleosides and to other modified nucleosides, such as 2′-O-methyl, O-propyl, and O-aminopropyl. Oligonucleotides having the 2′-MOE substituent also have been shown to be antisense inhibitors of gene expression with promising features for in vivo use (Martin, P., Helv. Chim. Acta, 1995, 78, 486-504; Altmann et al., Chimia, 1996, 50, 168-176: Altmann et al., Biochem. Soc. Trans., 1996, 24, 630-637; and Altmann et al., Nucleosides Nucleotides, 1997, 16,917-926).

In some aspects, 2′-sugar substituent groups are in either the arabino (up) position or ribo (down) position. In some aspects, a 2′-arabino modification is 2′-Farabino (FANA). Similar modifications can also be made at other positions on the sugar, particularly the 3′ position of the sugar on a 3′ terminal nucleoside or in 2′-5′ linked oligonucleotides and the 5′ position of 5′ terminal nucleotide.

In some aspects, suitable nucleosides have sugar surrogates such as cyclobutyl in place of the ribofuranosyl sugar. Representative U.S. patents that teach the preparation of such modified sugar structures include, but are not limited to, U.S.: 4,981,957: 5,118,800; 5,319,080; 5,359,044; 5,393,878; 5,446,137; 5,466,786; 5,514,785; 5,519,134: 5,567,811: 5,576.427; 5,591,722; 5,597,909; 5,610,300; 5,627,053: 5,639,873; 5,646,265; 5,658,873; 5,670,633; 5,792,747; and 5,700,920, each of which is herein incorporated by reference in its entirety.

In some aspects, nucleosides comprise a modification at the 2′-position of the sugar. In some aspects, nucleosides comprise a modification at the 5′-position of the sugar. In some aspects, nucleosides comprise modifications at the 2′-position and the 5′-position of the sugar. In some aspects, modified nucleosides may be useful for incorporation into oligonucleotides. In some aspects, modified nucleosides are incorporated into oligonucleosides at the 5′-end of the oligonucleotide.

b. Internucleoside linkages

Antisense oligonucleotides can optionally contain one or more modified internucleoside linkages. Two main classes of linking groups are defined by the presence or absence of a phosphorus atom. Representative phosphorus containing linkages include, but are not limited to, phosphodiesters (P═O), phosphotriesters, methylphosphonates, phosphoramidate, and phosphorothioates (P═S). Representative non-phosphorus containing linking groups include, but are not limited to, methylenemethylimino (—CH₂—N(CH₃)—O—CH₂), thiodiester (—O—C(O)—S—), thionocarbamate (—O—C(O)(NH)—S—); siloxane (—O—Si(H)₂—O—); and N,N′-dimethylhydrazine (CH₂—N(CH₃)—N(CH₃)—). Oligonucleotides having non phosphorus linking groups are referred to as oligonucleosides. Modified linkages, compared to natural phosphodiester linkages, can be used to alter, typically increase, nuclease resistance of the oligonucleotides. In some aspects, linkages having a chiral atom can be prepared as racemic mixtures, as separate enantiomers. Representative chiral linkages include, but are not limited to, alkylphosphonates and phosphorothioates. Methods of preparation of phosphorous-containing and non-phosphorous-containing linkages are well known to those skilled in the art.

The antisense oligonucleotides described herein can contain one or more asymmetric centers and thus give rise to enantiomers, diastereomers, and other stereoisomeric configurations that may be defined, in terms of absolute stereochemistry, as (R) or (S), such as for sugar anomers, or as (D) or (L) such as for amino acids et al. Antisense compounds provided herein can include all such possible isomers, as well as their racemic and optically pure forms.

In some aspects, antisense oligonucleotides have at least one modified internucleoside linkage. In some aspects, antisense oligonucleotides have at least 2 modified internucleoside linkages. In some aspects, antisense oligonucleotides have at least 3 modified internucleoside linkages. In some aspects, antisense oligonucleotides have at least 10 modified internucleoside linkages. In some aspects, each internucleoside linkage of an antisense oligonucleotide is a modified internucleoside linkage. In some aspects, such modified internucleoside linkages are phosphorothioate linkages.

c. Lengths

In some aspects, the present invention provides antisense oligonucleotides of any of a variety of ranges of lengths. In some aspects, antisense compounds or antisense oligonucleotides comprise or consist of X-Y linked nucleosides, where X and Y are each independently selected from 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and 50; provided that X-Y. For example, in some aspects, antisense compounds or antisense oligonucleotides comprise or consist of: 8-9, 8-10, 8-11, 8-12, 8-13, 8-14, 8-15, 8-16, 8-17, 8-18, 8-19, 8-20, 8-21, 8-22, 8-23, 8-24, 8-25, 8-26, 8-27, 8-28, 8-29, 8-30, 9-10, 9-11, 9-12, 9-13, 9-14, 9-15, 9-16, 9-17, 9-18, 9-19, 9-20, 9-21, 9-22, 9-23, 9-24, 9-25, 9-26, 9-27, 9-28, 9-29, 9-30, 10-11, 10-12, 10-13, 10-14, 10-15, 10-16, 10-17, 10-18, 10-19, 10-20, 10-21, 10-22, 10-23, 10-24, 10-25, 10-26, 10-27, 10-28, 10-29, 10-30, 11-12, 11-13, 11-14, 11-15, 11-16, 11-17, 11-18, 11-19, 11-20, 11-21, 11-22, 11-23, 11-24, 11-25, 11-26, 11-27, 11-28, 11-29, 11-30, 12-13, 12-14, 12-15, 12-16, 12-17, 12-18, 12-19, 12-20, 12-21, 12-22, 12-23, 12-24, 12-25, 12-26, 12-27, 12-28, 12-29, 12-30, 13-14, 13-15, 13-16, 13-17, 13-18, 13-19, 13-20, 13-21, 13-22, 13-23, 13-24, 13-25, 13-26, 13-27, 13-28, 13-29, 13-30, 14-15, 14-16, 14-17, 14-18, 14-19, 14-20, 14-21, 14-22, 14-23, 14-24, 14-25, 14-26, 14-27, 14-28, 14-29, 14-30, 15-16, 15-17, 15-18, 15-19, 15-20, 15-21, 15-22, 15-23, 15-24, 15-25, 15-26, 15-27, 15-28, 15-29, 15-30, 16-17, 16-18, 16-19, 16-20, 16-21, 16-22, 16-23, 16-24, 16-25, 16-26, 16-27, 16-28, 16-29, 16-30, 17-18, 17-19, 17-20, 17-21, 17-22, 17-23, 17-24, 17-25, 17-26, 17-27, 17-28, 17-29, 17-30, 18-19, 18-20, 18-21, 18-22, 18-23, 18-24, 18-25, 18-26, 18-27, 18-28, 18-29, 18-30, 19-20, 19-21, 19-22, 19-23, 19-24, 19-25, 19-26, 19-29, 19-28, 19-29, 19-30, 20-21, 20-22, 20-23, 20-24, 20-25, 20-26, 20-27, 20-28, 20-29, 20-30, 21-22, 21-23, 21-24, 21-25, 21-26, 21-27, 21-28, 21-29, 21-30, 22-23, 22-24, 22-25, 22-26, 22-27, 22-28, 22-29, 22-30, 23-24, 23-25, 23-26, 23-27, 23-28, 23-29, 23-30, 24-25, 24-26, 24-27, 24-28, 24-29, 24-30, 25-26, 25-27, 25-28, 25-29, 25-30, 26-27, 26-28, 26-29, 26-30, 27-28, 27-29, 27-30, 28-29, 28-30, or 29-30 linked nucleosides.

In some aspects, antisense compounds or antisense oligonucleotides are 15 nucleosides in length. In some aspects, antisense compounds or antisense oligonucleotides are 16 nucleosides in length. In some aspects, antisense compounds or antisense oligonucleotides are 17 nucleosides in length. In some aspects, antisense compounds or antisense oligonucleotides are 18 nucleosides in length. In some aspects, antisense compounds or antisense oligonucleotides are 19 nucleosides in length. In some aspects, antisense compounds or antisense oligonucleotides are 20 nucleosides in length.

d. Oligonucleotide Motifs

In some aspects, antisense oligonucleotides have chemically modified subunits arranged in specific orientations along their length. In some aspects, antisense oligonucleotides are fully modified. In some aspects, antisense oligonucleotides are uniformly modified. In some aspects, antisense oligonucleotides are uniformly modified and each nucleoside comprises a 2-MOE sugar moiety. In some aspects, antisense oligonucleotides are uniformly modified and each nucleoside comprises a 2′-OMe sugar moiety. In some aspects, antisense oligonucleotides are uniformly modified and each nucleoside comprises a morpholino sugar moiety.

In some aspects, oligonucleotides comprise an alternating motif. In some aspects, the alternating modification types are selected from among 2′-MOE, 2′-F, a bicyclic sugar-modified nucleoside, and DNA (unmodified 2′-deoxy). In some aspects, each alternating region comprises a single nucleoside.

In some aspects, oligonucleotides comprise one or more block of nucleosides of a first type and one or more block of nucleosides of a second type.

In some aspects, one or more alternating regions in an alternating motif include more than a single nucleoside of a type. For example, oligomeric compounds may include one or more regions of any of the following nucleoside motifs:

Nu1 Nu1 Nu2 Nu2 Nu1 Nu1;

Nu1 Nu2 Nu2 Nu1 Nu2 Nu2;

Nu1 Nu1 Nu2 Nu1 Nu1 Nu2;

Nu1 Nu2 Nu2 Nu1 Nu2 Nu1 Nu1 Nu2 Nu2;

Nu1 Nu2 Nu1 Nu2 Nu1 Nu1;

Nu1 Nu1 Nu2 Nu1 NU2 Nu1 Nu2;

Nu1 Nu2 Nu1 Nu2 Nu1 Nu1;

Nu1 Nu2 Nu2 Nu1 Nu1 Nu2 Nu2 Nu1 Nu2 Nu1 Nu2 Nu1 Nu1;

Nu2 Nu1 Nu2 Nu2 Nu1 Nu1 Nu2 Nu2 Nu1 Nu2 Nu1 Nu2 Nu1 Nu1; or

Nu1 Nu2 Nu1 Nu2 Nu2 Nu1 Nu1 Nu2 Nu2 Nu1 Nu2 Nu1 Nu2 Nu1 Nu1;

wherein Nu1 is a nucleoside of a first type and Nu2 is a nucleoside of a second type. In some aspects, one of Nu1 and Nu2 is a 2′-MOE nucleoside and the other of Nu1 and Nu2 is selected from: a 2′-OMe modified nucleoside, BNA, and an unmodified DNA or RNA nucleoside.

2. Oligomeric Compounds

In some aspects, oligomeric compounds are comprised only of an oligonucleotide. In some aspects, an oligomeric compound comprises an oligonucleotide and one or more conjugate and/or terminal groups. Such conjugate and/or terminal groups may be added to oligonucleotides having any of the chemical motifs described in this application. Thus, for example, an oligomeric compound comprising an oligonucleotide having one or more regions of alternating nucleosides may comprise a terminal group.

a. Conjugate Groups

In some aspects, oligonucleotides are modified by attachment of one or more conjugate groups. In general, conjugate groups modify one or more properties of the attached oligomeric compound including but not limited to, pharmacodynamics, pharmacokinetics, stability, binding, absorption, cellular distribution, cellular uptake, charge and clearance. Conjugate groups are routinely used in the chemical arts and are linked directly or via an optional conjugate linking moiety or conjugate linking group to a parent compound such as an oligomeric compound, such as an oligonucleotide. Conjugate groups can include without limitation, intercalators, reporter molecules, polyamines, polyamides, polyethylene glycols, thioethers, polyethers, cholesterols, thiocholesterols, cholic acid moieties, folate, lipids, phospholipids, biotin, phenazine, phenanthridine, anthraquinone, adamantane, acridine, fluoresceins, rhodamines, coumarins and dyes. Certain conjugate groups have been described previously, for example: cholesterol moiety (Letsinger et al., Proc. Natl. Acad. Sci. USA, 1989, 86, 6553-6556), cholic acid (Manoharan et al., Bioorg. Med. Chem. Let., 1994, 4, 1053-1060), a thioether, e.g., hexyl-S-tritylthiol (Manoharan et al., Ann. N.Y. Acad. Sci., 1992, 660, 306-309; Manoharan et al., Bioorg. Med. Chem. Let., 1993, 3, 2765-2770), athiocholesterol (Oberhauser et al., Nucl. Acids Res., 1992, 20, 533-538), an aliphatic chain, e.g., do-decan-diol or undecyl residues (Saison-Behmoaras et al., EMBO. J., 1991, 10, 1111-1118; Kabanov et al., FEBS Lett., 1990, 259, 327-330; Svinarchuk et al., Biochimie, 1993, 75, 49-54), a phospholipid, e.g., di-hexadecyl-rac-glycerol or triethyl-ammonium 1,2-di-O-hexadecyl-rac-glycero-3-H-phosphonate (Manoharan et al., Tetrahedron Lett., 1995, 36, 3651-3654; Shea et al., Nucl. Acids Res., 1990, 18, 3777-3783), a polyamine or a polyethylene glycol chain (Manoharan et al., Nucleosides & Nucleotides, 1995, 14,969-973), or adamantane acetic acid (Manoharan et al., Tetrahedron Lett., 1995, 36, 3651-3654), a palmityl moiety (Mishra et al., Biochim. Biophys. Acta, 1995, 1264. 229-237), or an octadecylamine or hexylamino-carbonyl-oxycholesterol moiety (Crooke et al., J. Pharmacol. Exp. Ther., 1996, 277, 923-937).

In some aspects, a conjugate group comprises an active drug substance, for example, aspirin, warfarin, phenylbutazone, ibuprofen, Suprofen, fen-bufen, ketoprofen, (S)-(+)-pranoprofen, carprofen, dansylsarcosine, 2,3,5-triiodobenzoic acid, flufenamic acid, folinic acid, a benzothiadiazide, chlorothiazide, a diazepine, indo-methicin, a barbiturate, a cephalosporin, a Sulfa drug, an antidiabetic, an antibacterial or an antibiotic. Oligonucleotide-drug conjugates and their preparation are described in U.S. patent application Ser. No. 09/334,130.

Representative U.S. patents that teach the preparation of oligonucleotide conjugates include, but are not limited to, U.S.: 4,828,979: 4,948,882: 5,218,105: 5,525,465; 5,541, 313; 5,545,730; 5,552,538; 5,578,717, 5,580,731: 5,580, 731: 5,591,584; 5,109,124; 5,118,802; 5,138,045; 5,414,077; 5,486,603: 5,512.439; 5,578,718; 5,608,046; 4,587,044; 4,605,735; 4,667,025; 4,762,779; 4,789,737; 4,824, 941; 4,835,263; 4,876,335; 4,904,582: 4,958,013; 5,082, 830; 5,112,963: 5,214,136; 5,082,830; 5,112,963: 5,214, 136: 5,245,022: 5,254,469; 5,258,506; 5,262,536; 5,272, 250; 5,292,873; 5,317,098: 5,371,241, 5,391,723; 5,416, 203, 5,451,463, 5,510,475; 5,512,667: 5,514,785: 5,565, 552; 5,567,810; 5,574,142; 5,585,481: 5,587,371; 5,595, 726; 5,597.696; 5,599,923; 5,599,928 and 5,688,941. Conjugate groups may be attached to either or both ends of an oligonucleotide (terminal conjugate groups) and/or at any internal position.

b. Terminal Groups

In some aspects, oligomeric compounds comprise terminal groups at one or both ends. In some aspects, a terminal group may comprise any of the conjugate groups described in this application. In some aspects, terminal groups may comprise additional nucleosides and/or inverted abasic nucleosides. In some aspects, a terminal group is a stabilizing group.

In some aspects, oligomeric compounds comprise one or more terminal stabilizing groups that enhance properties such as for example nuclease stability. Included in stabilizing groups are cap structures. The terms “cap structure” or “terminal cap moiety,” as used herein, refer to chemical modifications, which can be attached to one or both of the termini of an oligomeric compound. Certain terminal modifications protect oligomeric compounds having terminal nucleic acid moieties from exonuclease degradation, and can help in delivery and/or localization within a cell. The cap can be present at the 5′ terminus (5′-cap) or at the 3′-terminus (3′-cap) or can be present on both termini (for more non-limiting details see Wincott et al., International PCT publication No. WO 97/26270; Beaucage and Tyer, 1993, Tetrahedron 49, 1925: U.S. Patent Application Publication No. US 2005/0020525; and WO 03/004602).

In some aspects, one or more additional nucleosides are added to one or both terminal ends of an oligonucleotide of an oligomeric compound. Such additional terminal nucleosides are referred to herein as terminal-group nucleosides. In a double-stranded compound, such terminal-group nucleosides are terminal (3′ and/or 5′) overhangs. In the setting of double-stranded antisense compounds, such terminal-group nucleosides may or may not be complementary to a target nucleic acid. In some aspects, the terminal group is a non-nucleoside terminal group. Such non-terminal groups may be any terminal group other than a nucleoside.

c. Oligomeric Compound Motifs

In some aspects, oligomeric compounds comprise a motif: T-(Nu₁)ₙ₁,-(Nu₂)ₙ₂-(Nu₁)ₙ₃-(Nu₂)ₙ₄-(Nu₁)ₙ₅-T2, wherein:

Nu₁, is a nucleoside of a first type;

Nu₂, is a nucleoside of a second type:

each of n1 and n5 is, independently from 0 to 3:

the sum of n2 plus n4 is between 10 and 25:

n3 is from 0 and 5; and

each T₁ and T₂ is, independently, H, a hydroxyl protecting group, an optionally linked conjugate group or a capping group.

In some aspects, the Sum of n2 and n4 is 13 or 14; n1 is 2; n3 is 2 or 3; and n5 is 2. In some aspects, oligomeric compounds comprise a motif selected from Table A.

TABLE A

| n1 | n2 | n3 | n4 | n5 |
|---|---|---|---|---|
| 2 | 16 | 0 | 0 | 2 |
| 2 | 2 | 3 | 11 | 2 |
| 2 | 5 | 3 | 8 | 2 |
| 2 | 8 | 3 | 5 | 2 |
| 2 | 11 | 3 | 2 | 2 |
| 2 | 9 | 3 | 4 | 2 |
| 2 | 10 | 3 | 3 | 2 |
| 2 | 3 | 3 | 10 | 2 |
| 2 | 4 | 3 | 9 | 2 |
| 2 | 6 | 3 | 7 | 2 |
| 2 | 7 | 3 | 6 | 2 |
| 2 | 8 | 6 | 2 | 2 |
| 2 | 2 | 2 | 12 | 2 |
| 2 | 3 | 2 | 11 | 2 |
| 2 | 4 | 2 | 10 | 2 |
| 2 | 5 | 2 | 9 | 2 |
| 2 | 6 | 2 | 8 | 2 |
| 2 | 7 | 2 | 7 | 2 |
| 2 | 8 | 2 | 6 | 2 |
| 2 | 9 | 2 | 5 | 2 |
| 2 | 10 | 2 | 4 | 2 |
| 2 | 11 | 2 | 3 | 2 |
| 2 | 12 | 2 | 2 | 2 |

3. Antisense

In some aspects, oligomeric compounds are antisense compounds. Accordingly, in some aspects oligomeric compounds hybridize with a target nucleic acid (e.g., a target pre-mRNA or a target mRNA) resulting in an antisense activity.

a. Hybridization

In some aspects, antisense compounds specifically hybridize to a target nucleic acid when there is a sufficient degree of complementarity to avoid non-specific binding of the antisense compound to non-target nucleic acid sequences under conditions in which specific binding is desired (e.g., under physiological conditions in the case of in vivo assays or therapeutic treatment, and under conditions in which assays are performed in the case of in vitro assays).

Thus, “stringent hybridization conditions” or “stringent conditions” means conditions under which an antisense compounds hybridize to a target sequence, but to a minimal number of other sequences. Stringent conditions are sequence-dependent and will be different in different circumstances, and “stringent conditions” under which antisense oligonucleotides hybridize to a target sequence are determined by the nature and composition of the antisense oligonucleotides and the assays in which they are being investigated.

It is understood in the art that incorporation of nucleotide affinity modifications may allow for a greater number of mismatches compared to an unmodified compound. Similarly, certain nucleobase sequences may be more tolerant to mismatches than other nucleobase sequences. One of ordinary skill in the art is capable of determining an appropriate number of mismatches between oligonucleotides, or between an antisense oligonucleotide and a target nucleic acid, such as by determining melting temperature (Tm). Tm or ATm can be calculated by techniques that are familiar to one of ordinary skill in the art. For example, techniques described in Freier et al. (Nucleic Acids Research, 1997, 25, 22: 4429-4443) allow one of ordinary skill in the art to evaluate nucleotide modifications for their ability to increase the melting temperature of an RNA:DNA duplex.

b. Pre-mRNA Processing

In some aspects, antisense compounds provided herein are complementary to a pre-mRNA. In some aspects, such antisense compounds alter splicing of the pre-mRNA. In some aspects, the ratio of one variant of a mature mRNA corresponding to a target pre-mRNA to another variant of that mature mRNA is altered. In some aspects, the ratio of one variant of a protein expressed from the target pre-mRNA to another variant of the protein is altered. Certain oligomeric compounds and nucleobase sequences that may be used to alter splicing of a pre-mRNA may be found for example in U.S. Pat. Nos. 6,210,892; 5,627,274; 5,665,593; 5,916,808; 5,976,879; US2006/0172962; US2007/002390; US2005/0074801; US2007/0105807; US2005/0054836; WO 2007/090073; WO2007/047913, Hua et al., PLoS Biol 5(4):e73; Vickers et al., J. Immunol. 2006 Mar. 15; 176(6):3652-61; and Hua et al., American J. of Human Genetics (April 2008) 82, 1-15, each of which is hereby incorporated by reference in its entirety for any purpose. In some aspects antisense sequences that alter splicing are modified according to motifs described in this application.

In some aspects, ASOs or oligomeric compounds may include one or more modifications described in WO/2018/014043 (PCT/US2017/042465), WO/2018/014042 (PCT/US2017/042464), WO/2018/014041 (PCT/US2017/042463), the contents of which are incorporated herein in their entirety.

### Administration and Treatment

In some aspects, a “therapeutically effective” amount of a small molecule capable of increasing SMN function (e.g., Risdiplam or Branaplam), a recombinant SMN1 gene (e.g., in a viral vector, for example an rAAV), and/or a SMN2 ASO (e.g., nusinersen), are delivered to a subject as described herein (e.g., via concurrent or sequential administration) to achieve a desired result, for example, treatment of SMA or one or more symptoms thereof. In some aspects, SMA is assessed by clinical symptoms such as loss of body weight, decreased muscle strength, decreased muscle tone, presence of scoliosis, tremor or twitching, and/or decreased respiratory health. In some aspects, the SMA is assessed by age- and ability-appropriate motor function scales and electrophysiological measurement of motor unit health.

In some aspects, the motor neuron function of the subject can be tested by The Children's Hospital of Philadelphia Infant Test of Neuromuscular Disorders (CHOP INTEND) (e.g., Glanzman A M, et al. The Children's Hospital of Philadelphia Infant Test of Neuromuscular Disorders (CHOP INTEND): test development and reliability. Neuromuscul Disord. 2010; 20(3):155-161; Glanzman A M, Validation of the Children's Hospital of Philadelphia Infant Test of Neuromuscular Disorders (CHOP INTEND). Pediatr Phys Ther. 2011; 23(4):322-326, the contents relate to CHOP INTEND are incorporated herein by reference). In some aspects, the motor neuron function of subjects having later-onset SMA is assessed by the Hammersmith Functional Motor Scale-Expanded (HFMSE) (e.g., Glanzman A M et al; the Pediatric Neuromuscular Clinical Research Network for Spinal Muscular Atrophy (PNCR), and the Muscle Study Group (MSG). Validation of the Expanded Hammersmith Functional Motor Scale in spinal muscular atrophy type II and III. J Child Neurol. 2011; 26(12):1499-1507; The Pediatric Neuromuscular Clinical Research Network for SMA. Expanded Hammersmith Functional Motor Scale for SMA (HFMSE). Mar. 7, 2009, the contents relate to HFMSE are incorporated herein by reference). In some aspects, compound muscle action potential (CMAP) and/or motor unit number estimation (MUNE) is used to assess electrophysiological function of motor neuron. CAMP response is a measure of the electrophysiologic output from a specific muscle or muscle group following stimulation of the innervating nerve, which is described in Arnold W D, Sheth K A, et al. Electrophysiological motor unit number estimation (MUNE) measuring compound muscle action potential (CMAP) in mouse hindlimb muscles. J Vis Exp. 2015; 103:1-8), the contents of which is incorporated herein by reference. In some aspects, CMAP value decreases in subjects with SMA. In some aspect, CMAP decreases before the physical symptoms emerge. Motor unit number estimation (MUNE) is an electrophysiologic method to estimate the number of lower motor neurons innervating a group of muscles supplied by a nerve, and is well suited to assess motor neuron loss in SMA, which is described in Bromberg M B, Swoboda K J. Motor unit number estimation in infants and children with spinal muscular atrophy. Muscle Nerve. 2002; 25(3):445-447, the contents of which is described herein by reference. MUNE values are calculated from the ratio of the maximal compound muscle action potential (CMAP) to the average single motor unit potential (SMUP).

In some aspects, a desired result includes reducing muscle weakness, increasing muscle strength and tone, preventing or reducing scoliosis, or maintaining or increasing respiratory health, or reducing tremors or twitching. Other desired endpoints can be determined by a physician.

In some aspects, a small molecule for increasing SMN function (e.g., Risdiplam or Branaplam) and a recombinant SMN1 gene (e.g., in a rAAV) are administered (e.g., concurrently and sequentially) to a subject to increase body weight. In some aspects, a small molecule for increasing SMN function (e.g., Risdiplam or Branaplam) and a SMN2 ASO (e.g., nusinersen) are administered (e.g., concurrently and sequentially) to the subject to increase body weight. In some aspects, a small molecule for increasing SMN function (e.g., Risdiplam or Branaplam), a recombinant SMN1 gene (e.g., in a rAAV) and a SMN2 ASO are administered (e.g., concurrently and sequentially) to a subject to increase body weight. In some aspects, a small molecule for increasing SMN function (e.g., Risdiplam or Branaplam) and a recombinant SMN1 gene (e.g., in a rAAV) are administered (e.g., concurrently and sequentially) to the subject to prevent or reduce muscle weakness. In some aspects, a small molecule for increasing SMN function (e.g., Risdiplam or Branaplam) and a SMN2 ASO (e.g., nusinersen) are administered (e.g., concurrently and sequentially) to the subject to prevent or reduce muscle weakness. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to prevent or reduce muscle weakness. In some aspects, a small molecule for increasing SMN function (e.g., Risdiplam or Branaplam) and a recombinant SMN1 gene (e.g., in a rAAV) are administered (e.g., concurrently and sequentially) to the subject to increase muscle strength. In some aspects, a small molecule for increasing SMN function and a SMN2 ASO (e.g., nusinersen) are administered (e.g., concurrently and sequentially) to the subject to increase muscle strength. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to increase muscle strength. In some aspects, a small molecule for increasing SMN function (e.g., Risdiplam or Branaplam) and a recombinant SMN1 gene are administered (e.g., concurrently and sequentially) to the subject to increase muscle tone. In some aspects, a small molecule for increasing SMN function (e.g., Risdiplam or Branaplam) and a SMN2 ASO (e.g., nusinersen) are administered (e.g., concurrently and sequentially) to the subject to increase muscle tone. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to increase muscle tone. In some aspects, a small molecule for increasing SMN function and a recombinant SMN1 gene (e.g., in a rAAV) are administered (e.g., concurrently and sequentially) to the subject to prevent or reduce scoliosis. In some aspects, a small molecule for increasing SMN function (e.g., Risdiplam or Branaplam) and a SMN2 ASO (e.g., nusinersen) are administered (e.g., concurrently and sequentially) to the subject to prevent or reduce scoliosis. In some aspects, a small molecule for increasing SMN function, a recombinant SMN1 gene (e.g., in a rAAV) and a SMN2 ASO (e.g., nusinersen) are administered (e.g., concurrently and sequentially) to the subject to prevent or reduce scoliosis. In some aspects, a small molecule for increasing SMN function (e.g., Risdiplam or Branaplam) and a recombinant SMN1 gene (e.g., in a rAAV) are administered (e.g., concurrently and sequentially) to the subject to reduce tremors or twitching. In some aspects, a small molecule for increasing SMN function (e.g., Risdiplam or Branaplam) and a SMN2 ASO (e.g., nusinersen) are administered (e.g., concurrently and sequentially) to the subject to reduce tremors or twitching. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to reduce tremors or twitching. In some aspects, a small molecule for increasing SMN function (e.g., Risdiplam or Branaplam) and a recombinant SMN1 gene (e.g., in a rAAV) are administered (e.g., concurrently and sequentially) to the subject to maintain or increase respiratory health. In some aspects, a small molecule for increasing SMN function (e.g., Risdiplam or Branaplam) and a SMN2 ASO (e.g., nusinersen) are administered (e.g., concurrently and sequentially) to the subject to maintain or increase respiratory health. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to maintain or increase respiratory health. In some aspects, a small molecule for increasing SMN function (e.g., Risdiplam or Branaplam) and a recombinant SMN1 gene (e.g., in a rAAV) are administered (e.g., concurrently and sequentially) to the subject to prevent or reduce neuron loss. In some aspects, a small molecule for increasing SMN function (e.g., Risdiplam or Branaplam) and a SMN2 ASO (e.g., nusinersen) are administered (e.g., concurrently and sequentially) to the subject to prevent or reduce neuron loss. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to prevent or reduce neuron loss. In some aspects, a small molecule for increasing SMN function (e.g., Risdiplam or Branaplam) and a recombinant SMN1 gene (e.g., in a rAAV) are administered (e.g., concurrently and sequentially) to the subject to prevent or reduce motor neuron loss. In some aspects, a small molecule for increasing SMN function and a SMN2 ASO are administered (e.g., concurrently and sequentially) to the subject to prevent or reduce motor neuron loss. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to prevent or reduce motor neuron loss. In some aspects, small molecule for increasing SMN function (e.g., Risdiplam or Branaplam) and a recombinant SMN1 gene (e.g., in a rAAV) are administered (e.g., concurrently and sequentially) to the subject to improve the scores of any of the motor neuron function test and/or the electrophysiologic tests. In some aspects, a small molecule for increasing SMN function and a SMN2 ASO (e.g., nusinersen) are administered (e.g., concurrently and sequentially) to the subject to improve the scores of any of the motor neuron function test and/or the electrophysiologic tests. In some aspects, a small molecule for increasing SMN function (e.g., Risdiplam or Branaplam), a recombinant SMN1 gene (e.g., in a rAAV) and a SMN2 ASO (e.g., nusinersen) are administered (e.g., concurrently and sequentially) to the subject to improve the scores of any of the motor neuron function test and/or the electrophysiologic tests.

In some aspects, administration of a small molecule for increasing SMN function (e.g., Risdiplam or Branaplam) and a recombinant SMN1 gene (e.g., in a rAAV), or a small molecule for increasing SMN function and a SMN2 ASO (e.g., nusinersen), or a small molecule for increasing SMN function (e.g., Risdiplam or Branaplam), a recombinant SMN1 gene (e.g., in a rAAV) and a SMN2 ASO (e.g., nusinersen) produces a synergistic effect as measured by any of the tests described herein. In some aspects, the method described herein potentiates the effect of the small molecule that increases SMN function (e.g., Risdiplam or Branaplam) and allows for a lower dose of small molecule that increases SMN function (e.g., Risdiplam or Branaplam) to be administered to a subject. In some aspects, the method described herein potentiates the effect of the recombinant SMN1 gene (e.g., in a rAAV) and allows for a lower dose (e.g., a lower dose of rAAV encoding a recombinant SMN1 gene) to be delivered to a subject. In some aspects, the method described herein potentiates the effect of the SMN2 ASO (e.g., nusinersen) and allows for a lower dose of ASO (e.g., nusinersen) to be administered to a subject. In some aspects, a lower dose of rAAV encoding a recombinant SMN1 gene is less than 1×10¹⁰ GC. In some aspects, a lower dose of rAAV encoding a recombinant SMN1 gene is 1.0×10⁸ to 1.0×10¹⁰ GC. In some aspects, a lower dose of rAAV encoding a recombinant SMN1 gene is 1.0×10⁹ to 1.0×10¹⁰ GC. In some aspects, a lower dose of rAAV encoding a recombinant SMN1 gene is 1.0×10¹⁰ to 1.0×10¹³ GC. In some aspects, a lower dose of rAAV encoding a recombinant SMN1 gene administered to a human subject is 3×10¹³ GC. In some aspects, a lower dose of rAAV encoding a recombinant SMN1 gene administered to a human subject is less than 1×10¹⁴ GC, for example 1×10¹³ to 1×10¹⁴ GC, 1×10¹² to 1×10¹³ GC, 1×10¹¹ to 1×10¹² GC, 1×10¹⁰ to 1×10¹¹ GC, or 1×10⁹ to 1×10¹⁰ GC, or less per dose administered to the human subject. In some aspects, a lower dose of SMN2 ASO (e.g., nusinersen) is 12 mg. A total of 5 mg to 60 mg per dose of SMN2 ASO (e.g., nusinersen) is administered to the subject. In some aspects, a total of 12 mg to 48 mg per dose of SMN2 ASO (e.g., nusinersen) is administered to the subject. In some aspects, a total of 12 mg to 36 mg per dose of SMN2 ASO (e.g., nusinersen) is administered to the subject. In some aspects, a total of 12 mg per dose of SMN2 ASO (e.g., nusinersen) is administered to the subject.

In some instances, SMA is detected in a fetus at around 30 to 36 weeks of pregnancy. In this situation, it may be desirable to treat the neonate as soon as possible after delivery. It also may be desirable to treat the fetus in utero. Thus, a method of rescuing and/or treating a neonatal subject having SMA is provided, comprising the step of administering (e.g., concurrently and sequentially), a small molecule for increasing SMN function (e.g., Risdiplam or Branaplam) and a recombinant SMN1 gene (e.g., in a rAAV), or a small molecule for increasing SMN function (e.g., Risdiplam or Branaplam) and a SMN2 ASO (e.g., nusinersen), or a small molecule for increasing SMN function (e.g., Risdiplam or Branaplam), a recombinant SMN1 gene (e.g., in a rAAV) and a SMN2 ASO (e.g., nusinersen) to the neuronal cells of a fetus and/or a newborn subject (e.g., a human fetus and/or newborn). In some aspects, a method of rescuing and/or treating a fetus having SMA is provided, comprising the step of administering (e.g., concurrently and sequentially), a small molecule for increasing SMN function (e.g., Risdiplam or Branaplam) and a recombinant SMN1 gene (e.g., in a rAAV), or a small molecule for increasing SMN function and a SMN2 ASO (e.g., nusinersen), or a small molecule for increasing SMN function (e.g., Risdiplam or Branaplam), a recombinant SMN1 gene and a SMN2 ASO (e.g., nusinersen) to the neuronal cells of the fetus in utero. In some aspects, the method comprises administering (e.g., concurrently and sequentially), one or more compositions described herein via intrathecal injection. In some aspects, treatment in utero is defined as administering (e.g., concurrently and sequentially), a small molecule for increasing SMN function (e.g., Risdiplam or Branaplam) and a recombinant SMN1 gene (e.g., in a rAAV), or a small molecule for increasing SMN function (e.g., Risdiplam or Branaplam) and a SMN2 ASO (e.g., nusinersen), or a small molecule for increasing SMN2 function (e.g., Risdiplam or Branaplam), a recombinant SMN1 gene (e.g., in a rAAV) and a SMN2 ASO (e.g., nusinersen) as described herein after detection of SMA in the fetus. See, e.g., David et al, Recombinant adeno-associated virus-mediated in utero gene transfer gives therapeutic transgene expression in the sheep, Hum Gene Ther. 2011 April; 22(4):419-26. doi: 10.1089/hum.2010.007. Epub 2011 Feb. 2, which is incorporated herein by reference.

In some aspects, neonatal treatment involves delivering at least one dose of a small molecule for increasing SMN function (e.g., Risdiplam or Branaplam) and a recombinant SMN1 gene (e.g., in a rAAV), or a small molecule for increasing SMN2 function (e.g., Risdiplam or Branaplam) and a SMN2 ASO (e.g., nusinersen), or a combination of a small molecule for increasing SMN function (e.g., Risdiplam or Branaplam), a recombinant SMN1 gene (e.g., in a rAAV) and a SMN2 ASO (e.g., nusinersen) within 8 hours, the first 12 hours, the first 24 hours, or the first 48 hours of delivery. In another aspect, particularly for a primate (human or non-human), neonatal delivery is within the period of about 12 hours to about 1 week, 2 weeks, 3 weeks, or about 1 month, or after about 24 hours to about 48 hours.

In some aspects, for late onset SMA, a small molecule for increasing SMN function (e.g., Risdiplam or Branaplam) and a recombinant SMN1 gene (e.g., in a rAAV), or a combination of a small molecule for increasing SMN function (e.g., Risdiplam or Branaplam) and a SMN2 ASO (e.g., nusinersen), or a combination of a small molecule for increasing SMN function (e.g., Risdiplam or Branaplam), a recombinant SMN1 gene (e.g., in a rAAV) and a SMN2 ASO (e.g., nusinersen) are administered after onset of symptoms. In some aspects, treatment of the patient (e.g., a first injection) is initiated prior to the first year of life. In another aspect, treatment is initiated after the first 1 year, or after the first 2 to 3 years of age, after 5 years of age, after 11 years of age, or at an older age.

In some aspects, a small molecule for increasing SMN2 function and a recombinant SMN1 gene (e.g., in a rAAV), or a small molecule for increasing SMN function (e.g., Risdiplam or Branaplam) and a SMN2 ASO (e.g., nusinersen), or a small molecule for increasing SMN function (e.g., Risdiplam or Branaplam), a recombinant SMN1 gene (e.g., in a rAAV) and a SMN2 ASO (e.g., nusinersen) are re-administered at a later date.

In some aspects, more than one re-administration is provided. Such re-administration may involve re-administering a recombinant SMN1 gene in the same type of viral vector, a different viral vector (e.g., using AAV capsid proteins of a different serotype), or via non-viral delivery. For example, in the event a patient was treated with a first rAAV (e.g., rAAV9) encoding SMN1 and requires a second treatment with a recombinant SMN1 gene (e.g., in addition to receiving a small molecule for increasing SMN function (e.g., Risdiplam or Branaplam) or a small molecule and a SMN2 ASO), a second different rAAV (e.g., rAAVhu68) encoding the recombinant SMN1 gene can be subsequently administered, and vice-versa. Also, if a patient has neutralizing antibodies to a first rAAV serotype, then a second different rAAV serotype can be used to deliver a second dose of a recombinant SMN1 gene to a subject.

In some aspects, treatment of SMA patients with a small molecule for increasing SMN function (e.g., Risdiplam or Branaplam) and a recombinant SMN1 gene (e.g., in a rAAV), or a small molecule for increasing SMN function (e.g., Risdiplam or Branaplam) and a SMN2 ASO (e.g., nusinersen), or a small molecule for increasing SMN function (e.g., Risdiplam or Branaplam), a recombinant SMN1 gene (e.g., in a rAAV) and a SMN2 ASO may require a further therapy, such as transient co-treatment with an immunosuppressant before, during and/or after treatment with compositions described in this application.

Immunosuppressants for such co-therapy include, but are not limited to, steroids, antimetabolites, T-cell inhibitors, and alkylating agents, or procedures to remove circulating antibodies such as plasmapheresis. For example, such transient treatment may include a steroid (e.g., prednisone, or prednisolone) dosed once daily for 7 days at a decreasing dose, in an amount starting at about 60 mg, and decreasing by 10 mg/day (day 7 no dose). Other doses and immunosuppressants may be selected.

In some aspects, a subject has one or more indicators of SMA. In some aspects, the subject has reduced electrical activity of one or more muscles. In some aspects, the subject has a mutant SMN1 gene (e.g., two mutant alleles of the SMN1 gene). In some aspects, the subject's SMN1 gene (e.g., both alleles of the SMN1 gene) is absent or incapable of producing functional SMN protein. In some aspects the subject has a deletion or a loss of function point mutation in each SMN1 allele. In some aspects the subject is homozygous for a SMN1 gene mutation. In some aspects, the subject is diagnosed by a genetic test. In some aspects, the subject is identified by muscle biopsy. In some aspects, a subject is unable to sit upright. In some aspects, a subject is unable to stand or walk. In some aspects, a subject requires assistance to breathe and/or eat. In some aspects, a subject is identified by electrophysiological measurement of muscle and/or muscle biopsy.

In some aspects, the subject has SMA type I. In some aspects, the subject has SMA type II. In some aspects, the subject has SMA type III. In some aspects, the subject is diagnosed as having SMA in utero. In some aspects, the subject is diagnosed as having SMA within one week after birth. In some aspects, the subject is diagnosed as having SMA within one month of birth. In some aspects, the subject is diagnosed as having SMA by 3 months of age. In some aspects, the subject is diagnosed as having SMA by 6 months of age. In some aspects, the subject is diagnosed as having SMA by 1 year of age. In some aspects, the subject is diagnosed as having SMA between 1 and 2 years of age. In some aspects, the subject is diagnosed as having SMA between 1 and 15 years of age. In some aspects, the subject is diagnosed as having SMA when the subject is older than 15 years of age.

In some aspects, the first dose of a pharmaceutical composition (e.g., of a small molecule for increasing SMN function (e.g., Risdiplam or Branaplam), a recombinant SMN1 gene (e.g., in a rAAV), a SMN2 ASO (e.g., nusinersen), or both) is administered in utero. In some such aspects, the first dose is administered before complete development of the blood-brain-barrier. In some aspects, the first dose is administered to the subject in utero systemically. In some aspects, the first dose is administered in utero after formation of the blood-brain-barrier. In some aspects, the first dose is administered to the CSF.

In some aspects, the first dose of a pharmaceutical composition (e.g., of a small molecule for increasing SMN function such as Risdiplam or Branaplam, a recombinant SMN1 gene (e.g., in a rAAV), a SMN2 ASO (e.g., nusinersen), or both) is administered when the subject is less than one week old. In some aspects, the first dose is administered when the subject is less than one month old. In some aspects, the first dose is administered when the subject is less than 3 months old. In some aspects, the first dose is administered when the subject is less than 6 months old. In some aspects, the first dose is administered when the subject is less than one year old. In some aspects, the first dose is administered when the subject is less than 2 years old. In some aspects, the first dose is administered when the subject is less than 15 years old. In some aspects, the first dose is administered when the subject is older than 15 years old.

In some aspects, a small molecule for increasing SMN function (e.g., Risdiplam or Branaplam), and/or a SMN2 ASO (e.g., nusinersen), is administered 1-6 times per year, and the recombinant SMN1 gene (e.g., in a rAAV) is administered once initially. In some aspects, two or more subsequent doses of a small molecule for increasing SMN function (e.g., Risdiplam or Branaplam), and/or SMN2 ASO (e.g., nusinersen) are administered following an initial administration of a small molecule for increasing SMN function (e.g., Risdiplam or Branaplam), SMN2 ASO (e.g., nusinersen) and recombinant SMN1 gene (e.g., in a rAAV). In some aspects, the SMN2 ASO (e.g., nusinersen) is administered twice monthly. In some aspects, such doses are administered every month. In some aspects, the SMN2 ASO (e.g., nusinersen) is administered every 2 months. In some aspects, the SMN2 ASO (e.g., nusinersen) is administered every 6 months. In some aspects, the recombinant SMN1 gene (e.g., in an rAAV) is re-administered, for example 1 or more years (e.g., 2-5 years, 5-10 years, 10-15 years, 15-20 years on longer) after an initial administration.

In some aspects, administration of at least one pharmaceutical composition (e.g., of a small molecule for increasing SMN function (e.g., Risdiplam or Branaplam), a recombinant SMN1 gene (e.g., in a rAAV), and/or a SMN2 ASO (e.g., nusinersen) results in a phenotypic change in the subject. In some aspects, such phenotypic changes include, but are not limited to: increased absolute amount of recombinant SMN mRNA and/or cellular SMN mRNA that includes exon 7; increase in the ratio SMN mRNA that includes exon 7 to SMN mRNA lacking exon 7; increased absolute amount of SMN protein that includes exon 7; increase in the ratio of SMN protein that includes exon 7 to SMN protein lacking exon 7; improved muscle strength; improved electrical activity in at least one muscle; improved respiration; weight gain; and survival. In some aspects, at least one phenotypic change is detected in a motor neuron of the subject. In some aspects, administration of at least one pharmaceutical composition described in this application results in a subject being able to sit-up, to stand, and/or to walk. In some aspects, administration of at least one pharmaceutical composition results in a subject being able to eat, drink, and/or breathe without assistance. In some aspects, efficacy of treatment is assessed by electrophysiological assessment of muscle. In some aspects, administration of a pharmaceutical composition improves at least one symptom of SMA and has little or no inflammatory effect. In some aspects, absence of inflammatory effect is determined by the absence of significant increase in Aif1 levels upon treatment.

In some aspects, administration of at least one pharmaceutical composition delays the onset of at least one symptom of SMA. In some aspects, administration of at least one pharmaceutical composition slows the progression of at least one symptom of SMA. In some aspects, administration of at least one pharmaceutical composition reduces the severity of at least one symptom of SMA. In some aspects, administration of at least one pharmaceutical composition results in an undesired side-effect. In some aspects, a treatment regimen is identified that results in desired amelioration of symptoms while avoiding undesired side-effects.

### Dosage and Formulation

Accordingly, in some aspects, a therapeutically effective amount of a SMN2 ASO (e.g., nusinersen) is administered to a subject that has SMA. In some aspects the SMN2 ASO (e.g., nusinersen) is administered alone to the subject. In some aspects, the SMN2 ASO (e.g., nusinersen) is administered to the subject along with other compounds and/or pharmaceutical compositions. In some aspects, a SMN2 ASO (e.g., nusinersen) and a recombinant nucleic acid (e.g., in an rAAV), or a SMN2 ASO (e.g., nusinersen) and a small molecule for increasing SMN function (e.g., Risdiplam or Branaplam) are administered to the subject. In some aspects, a small molecule for increasing SMN function (e.g., Risdiplam or Branaplam), the SMN2 ASO (e.g., nusinersen), and/or the recombinant nucleic acid encoding SMN1 (e.g., in an rAAV) are administered concurrently (e.g., simultaneously or during the same medical visit), or sequentially (e.g., during different medical visits) to the subject. In some aspects, the small molecule that increases SMN function (e.g., Risdiplam or Branaplam), the SMN2 ASO (e.g., nusinersen) and the recombinant nucleic acid are administered separately to the subject.

In some aspects, the small molecule for increasing SMN function (e.g., Risdiplam or Branaplam) and the recombinant nucleic acid encoding SMN1 (e.g., in a rAAV) are administered to a subject concurrently (e.g., either simultaneously or at different times during a visit to a hospital, clinic, or other medical center, for example at different times during the same day of a medical visit). In some aspects, the small molecule for increasing SMN function (e.g., Risdiplam or Branaplam) and the SMN2 ASO (e.g., nusinersen) are administered to a subject concurrently. In some aspects, the small molecule for increasing SMN function (e.g., Risdiplam or Branaplam), the recombinant nucleic acid encoding SMN1 (e.g., in a rAAV) and the SMN2 ASO (e.g., nusinersen) are administered to a subject concurrently Accordingly, in some aspects, administering the small molecule for increasing SMN function (e.g., Risdiplam or Branaplam), the SMN2 ASO (e.g., nusinersen) and the recombinant nucleic acid encoding SMN1 concurrently means administration during the same medical visit (e.g., during the same clinic day). In some aspects, administering the small molecule for increasing SMN function (e.g., Risdiplam or Branaplam), the SMN2 ASO (e.g., nusinersen) and the recombinant nucleic acid encoding SMN1 concurrently means administration at different times during the same visit (e.g., during the same clinic day). In some aspects, the concurrent administration of the small molecule for increasing SMN function (e.g., Risdiplam or Branaplam), SMN1 gene (e.g., in a rAAV) and the SMN2 ASO (e.g., nusinersen) is an initiation of a new therapy. In other aspects, the concurrent administration of the small molecule for increasing SMN function (e.g., Risdiplam or Branaplam), SMN1 gene (e.g., in a rAAV) and the SMN2 ASO (e.g., nusinersen) is an additional therapy for a subject currently being treated with a different composition.

In some aspects, the small molecule for increasing SMN function (e.g., Risdiplam or Branaplam) and the recombinant nucleic acid encoding SMN1 (e.g., in a rAAV) are administered to a subject sequentially during different visits (e.g., different clinic days). In some aspects, the small molecule for increasing SMN function (e.g., Risdiplam or Branaplam) and the SMN2 ASO (e.g., nusinersen) are administered to a subject sequentially during different visits (e.g., different clinic days). In some aspects, the small molecule for increasing SMN function (e.g., Risdiplam or Branaplam), the recombinant nucleic acid encoding SMN1 and the SMN2 gene (e.g., in a rAAV) are administered to a subject sequentially during different visits (e.g., different clinic days). In some aspects, administering the small molecule for increasing SMN function (e.g., Risdiplam or Branaplam), the SMN2 ASO (e.g., nusinersen) and the recombinant nucleic acid encoding SMN1 sequentially means administration of recombinant nucleic acid encoding SMN1 (e.g., in a rAAV) during a first visit, followed by administration of small molecule and/or SMN2 ASO (e.g., nusinersen) during a different visit (e.g., different clinic days). In some aspects, administering the small molecule for increasing SMN function (e.g., Risdiplam or Branaplam), the SMN2 ASO (e.g., nusinersen) and the recombinant nucleic acid encoding SMN1 sequentially means administration of SMN2 ASO (e.g., nusinersen) during a first visit, followed by administration of the small molecule that increases SMN function (e.g., Risdiplam or Branaplam) and/or recombinant nucleic acid encoding SMN1 (e.g., in a rAAV) during a different visit (e.g., different clinic days). In some aspects, administering the small molecule for increasing SMN function (e.g., Risdiplam or Branaplam), the SMN2 ASO (e.g., nusinersen) and the recombinant nucleic acid encoding SMN1 sequentially means administration of small molecule that increases SMN function (e.g., Risdiplam or Branaplam) during a first visit, followed by administration of the recombinant nucleic acid encoding SMN1 and/or SMN2 ASO (e.g., nusinersen) during a different visit (e.g., different clinic days). In some aspects, the small molecule for increasing SMN function (e.g., Risdiplam or Branaplam), the recombinant nucleic acid encoding SMN1 and the SMN2 ASO (e.g., nusinersen) are administered at different frequencies. As used herein, a sequential administration can include an administration protocol wherein an administration of a first therapy (e.g., small molecule for increasing SMN2 function such as Risdiplam or Branaplam) during a medical visit can follow or precede one or more administrations of a second therapy (e.g., a SMN2 ASO (e.g., nusinersen) and/or recombinant nucleic acid encoding SMN1 (e.g., in a rAAV) or the combination thereof) during one or more different medical visits.

In some aspects, the small molecule for increasing SMN2 function (e.g., Risdiplam or Branaplam), the SMN2 ASO (e.g., nusinersen) and the recombinant SMN1 gene (e.g., in a rAAV) are administered at different frequencies. In some aspects, the SMN2 ASO (e.g., nusinersen) or the small molecule for increasing SMN2 function (e.g., Risdiplam or Branaplam) is administered to the subject 1-6 times per year. In some aspects, the recombinant SMN1 gene (e.g., in a rAAV) is administered once. In some aspects, two or more subsequent doses of the small molecule for increasing SMN2 function (e.g., Risdiplam or Branaplam) and/or the SMN2 ASO (e.g., nusinersen) are administered following an initial administration of the SMN2 ASO (e.g., nusinersen) and recombinant SMN1 gene. In some aspects, the SMN2 ASO (e.g., nusinersen) is administered to the subject prior to the administration of the small molecule for increasing SMN2 function (e.g., Risdiplam or Branaplam), SMN2 ASO and/or recombinant SMN1 gene (e.g., in a rAAV). In some aspects, the SMN2 ASO (e.g., nusinersen) is administered to the subject at a dose of 0.01 to 25 milligrams (e.g., 0.01 to 10 milligrams, 0.05 to 5 milligrams, 0.1 to 2 milligrams, or 0.5 to 1 milligrams) per kilogram of body weight of the subject, and the recombinant SMN1 gene (e.g., in a rAAV) is administered in an rAAV at a dose from 2×10¹⁰ to 2×10¹⁴ GC (e.g., from 1.0×10¹³ to 1.0×10¹⁴ GC, or for example for IT dosing from about 1.0×10¹³ to 5.0×10¹⁴ GC). In some aspects, the SMN2 ASO is administered to the subject at a dose of 0.001 to 25 milligrams (e.g., 0.001 to 10 milligrams, 0.005 to 5 milligrams, 0.01 to 2 milligrams, or 0.05 to 1 milligrams) per kilogram of body weight of the subject, and the recombinant SMN1 gene (e.g., in a rAAV) is administered in an rAAV at a dose from 1×10¹⁰ to 2×10¹⁴ GC (e.g., from 1.0×10¹³ to 1.0×10¹⁴ GC, or for example for IT dosing from about 1.0×10¹³ to 5.0×10¹⁴ GC) or for example for IV dosing from about 3×10¹³ to 5×10¹⁴ GC. In some aspects, the SMN2 ASO (e.g., nusinersen) is administered at a dose from 0.01 to 10 milligrams per kilogram of body weight of the subject. In some aspects, the SMN2 ASO (e.g., nusinersen) is administered at a dose from 0.001 to 10 milligrams per kilogram of body weight of the subject. In some aspects, the SMN2 ASO (e.g., nusinersen) is administered at a dose of less than 0.001 milligrams per kilogram of body weight of the subject.

In some aspects, a total of 5 mg to 60 mg per dose of SMN2 ASO (e.g., nusinersen) is administered to the subject. In some aspects, a total of 5 mg to 20 mg per dose of SMN2 ASO (e.g., nusinersen) is administered to the subject. In some aspects, a total of 12 mg to 48 mg per dose of SMN2 ASO (e.g., nusinersen) is administered to the subject. In some aspects, a total of 12 mg to 36 mg per dose of SMN2 ASO (e.g., nusinersen) is administered to the subject. In some aspects, a total of 28 mg per dose of SMN2 ASO (e.g., nusinersen) is administered to the subject. In some aspects, a total of 12 mg per dose of SMN2 ASO (e.g., nusinersen) is administered to the subject. In some aspects, the SMN2 ASO (e.g., nusinersen) and/or the recombinant SMN1 gene is administered to the subject intravenously or intramuscularly. In some aspects, the SMN2 ASO (e.g., nusinersen) and/or the recombinant SMN1 gene is administered into the intrathecal space of the subject. In some aspects, the SMN2 ASO (e.g., nusinersen) and/or the recombinant SMN1 gene is administered into the intracisternal magna space of the subject. In some aspects, administration of the SMN2 ASO (e.g., nusinersen) and the recombinant nucleic acid increase intracellular SMN protein level in the subject. In some aspects, administration of the SMN2 ASO (e.g., nusinersen) and the recombinant nucleic acid increase intracellular SMN protein level in the cervical, thoracic, and lumbar spinal cord segments of motor neurons in the subject.

In some aspects, doses of the small molecule for increasing SMN2 function (e.g., Risdiplam or Branaplam), recombinant SMN1 gene (e.g., in an rAAV) and SMN2 ASO (e.g., nusinersen) are administered by bolus injection into the CSF. In some aspects, doses are administered by LP and/or ICM bolus injection. In some aspects, doses are administered by bolus systemic injection (e.g., subcutaneous, intramuscular, or intravenous injection). In some aspects, subjects receive bolus injections into the CSF and bolus systemic injections. In some aspects, the doses of the CSF bolus and the systemic bolus may be the same or different from one another. In some aspects, the CSF and systemic doses are administered at different frequencies.

In some aspects, pharmaceutical compositions comprising a small molecule for increasing SMN2 function (e.g., Risdiplam or Branaplam), a recombinant SMN1 gene (e.g., in an rAAV), and/or a SMN2 ASO (e.g., nusinersen) are provided. Pharmaceutical compositions can be designed for delivery to subjects in need thereof by any suitable route (e.g., by different routes suitable for each therapy). For example, one or more compositions may be administered to human subjects using routes comprising intracerebroventricular (ICV), intravenous (IV), and intrathecal (IT) (e.g., via lumbar puncture (LP), and/or intracisternal magna (ICM) delivery).

In some aspects, direct delivery to the CNS is desired and may be performed via intrathecal injection. The term “intrathecal administration” refers to delivery that targets the cerebrospinal fluid (CSF). This may be done by direct injection into the ventricular or lumbar CSF, by suboccipital puncture, or by other suitable means. Meyer et al, Molecular Therapy (31 Oct. 2014), demonstrated the efficacy of direct CSF injection which resulted in widespread transgene expression throughout the spinal cord in mice and nonhuman primates when using a 10 times lower dose compared to the IV application. This document is incorporated herein by reference. In some aspects, a recombinant SMN1 gene is delivered via intracerebroventricular viral injection (see, e.g., Kim et al, J Vis Exp. 2014 Sep. 15; (91):51863, which is incorporated herein by reference). See also, Passini et al, Hum Gene Ther. 2014 July; 25(7):619-30, which is incorporated herein by reference. In some aspects, a composition is delivered via lumbar injection.

In some aspects, delivery means and formulations are designed to avoid direct systemic delivery of a suspension containing AAV composition(s) described in this application. Suitably, this may have the benefit of reducing systemic exposure as compared to systemic administration, reducing toxicity and/or reducing undesirable immune responses to the AAV and/or transgene product.

Compositions comprising a small molecule for increasing SMN2 function (e.g., Risdiplam or Branaplam), a recombinant SMN1 gene (e.g., in an rAAV) and/or SMN2 ASO (e.g., nusinersen) may be formulated for any suitable route of administration (e.g., oral, inhalation, intranasal, intratracheal, intraarterial, intraocular, intravenous, intramuscular, and other parenteral routes).

In some aspects, recombinant SMN1 gene delivery constructs described in this application may be delivered in a single composition or multiple compositions. In some aspects, two or more different AAV may be delivered (see, e.g., WO 2011/126808 and WO 2013/049493). In some aspects, such multiple viruses may contain different replication-defective viruses (e.g., AAV, adenovirus, and/or lentivirus). Alternatively, delivery may be mediated by non-viral constructs, e.g., “naked DNA”, “naked plasmid DNA”, RNA, and mRNA, coupled with various delivery compositions and nano particles, including, e.g., micelles, liposomes, cationic lipid—nucleic acid compositions, poly-glycan compositions and other polymers, lipid and/or cholesterol-based—nucleic acid conjugates, and other constructs such as described in this application or known in the art. See, e.g., X. Su et al, Mol. Pharmaceutics, 2011, 8 (3), pp 774-787; web publication: Mar. 21, 2011; WO2013/182683, WO 2010/053572 and WO 2012/170930, both of which are incorporated herein by reference. Non-viral SMN1 delivery constructs also may be formulated for any suitable route of administration.

Viral vectors, or non-viral DNA or RNA transfer moieties, can be formulated with a physiologically acceptable carrier for use in gene transfer and gene therapy applications. A number of suitable purification methods may be selected. Examples of suitable purification methods for separating empty capsids from vector particles are described, e.g., the process described in International Patent Application No. PCT/US 16/65976, filed Dec. 9, 2016 and its priority documents US Patent Application Nos. 62/322,098, filed Apr. 13, 2016 and U.S. Patent Appln No. 62/266,341, filed on Dec. 11, 2015, and entitled “Scalable Purification Method for AAV8”, which is incorporated by reference herein. See, also, purification methods described in International Patent Application No. PCT/US 16/65974, filed Dec. 9, 2016, and its priority documents, U.S. Patent Applications No. 62/322,083, filed Apr. 13, 2016 and 62/266,351, filed Dec. 11, 2015 (AAV1); International Patent Appln No. PCT/US16/66013, filed Dec. 9, 2016 and its priority documents U.S. Provisional Applications No. 62/322,055, filed Apr. 13, 2016 and 62/266,347, filed Dec. 11, 2015 (AAVrhlO); and International Patent Application No. PCT/US 16/65970, filed Dec. 9, 2016, and its priority applications U.S. Provisional Application Nos. 62/266,357 and 62/266,357 (AAV9), which are incorporated by reference herein. Briefly, a two-step purification scheme is described which selectively captures and isolates the genome-containing rAAV vector particles from the clarified, concentrated supernatant of a rAAV production cell culture. The process utilizes an affinity capture method performed at a high salt concentration followed by an anion exchange resin method performed at high pH to provide rAAV vector particles which are substantially free of rAAV intermediates.

In the case of AAV viral vectors, quantification of the genome copies (“GC”) may be used as the measure of the dose contained in the formulation. Any method known in the art can be used to determine the genome copy (GC) number of the replication-defective virus compositions of the invention. One method for performing AAV GC number titration is as follows: Purified AAV vector samples are first treated with DNase to eliminate contaminating host DNA from the production process. The DNase resistant particles are then subjected to heat treatment to release the genome from the capsid. The released genomes are then quantitated by real-time PCR using primer/probe sets targeting specific region of the viral genome (for example poly A signal). Another suitable method for determining genome copies are the quantitative-PCR (qPCR), particularly the optimized qPCR or digital droplet PCR (Lock Martin, et al, Human Gene Therapy Methods. April 2014, 25(2): 115-125. doi: 10.1089/hgtb.2013.131, published online ahead of editing Dec. 13, 2013).

In some aspects, replication-defective virus compositions can be formulated in dosage units to contain an amount of replication-defective virus that is in the range of about 1.0×10⁹ GC to about 1.0×10¹⁵ GC (e.g., to treat an average subject of 70 kg in body weight) including all integers or fractional amounts within the range, and preferably 1.0×10¹² GC to 1.0×10¹⁴ GC for a human patient. The total dose administered to a subject may depend on the route of administration. In some aspects, the compositions are formulated to contain at least 1×10⁹, 2×10⁹, 3×10⁹, 4×10⁹, 5×10⁹, 6×10⁹, 7×10⁹, 8×10⁹, or 9×10⁹ GC per dose including all integers or fractional amounts within the range. In another aspect, the compositions are formulated to contain at least 1×10¹⁰, 2×10¹⁰, 3×10¹⁰, 4×10¹⁰, 5×10¹⁰, 6×10¹⁰, 7×10¹⁰, 8×10¹⁰, or 9×10¹⁰ GC per dose including all integers or fractional amounts within the range. In another aspect, the compositions are formulated to contain at least 1×10¹¹, 2×10¹¹, 3×10¹¹, 4×10¹¹, 5×10¹¹, 6×10¹¹, 7×10¹¹, 8×10¹¹, or 9×10¹¹ GC per dose including all integers or fractional amounts within the range. In another aspect, the compositions are formulated to contain at least 1×10¹², 2×10¹², 3×10¹², 4×10¹², 5×10¹², 6×10¹², 7×10¹², 8×10¹², or 9×10¹² GC per dose including all integers or fractional amounts within the range. In another aspect, the compositions are formulated to contain at least 1×10¹³, 2×10¹³, 3×10¹³, 4×10¹³, 5×10¹³, 6×10¹³, 7×10¹³, 8×10¹³, or 9×10¹³ GC per dose including all integers or fractional amounts within the range. In another aspect, the compositions are formulated to contain at least 1×10¹⁴, 2×10¹⁴, 3×10¹⁴, 4×10¹⁴, 5×10¹⁴, 6×10¹⁴, 7×10¹⁴, 8×10¹⁴, or 9×10¹⁴ GC per dose including all integers or fractional amounts within the range. In another aspect, the compositions are formulated to contain at least 1×10¹⁵, 2×10¹⁵, 3×10¹⁵, 4×10¹⁵, 5×10¹⁵, 6×10¹⁵, 7×10¹⁵, 8×10¹⁵, or 9×10¹⁵ GC per dose including all integers or fractional amounts within the range. In some aspects, for human application the dose of a virus (e.g., of an rAAV) can range from 1×10¹⁰ to about 1×10¹² GC per dose including all integers or fractional amounts within the range.

These above doses may be administered in a variety of volumes of pharmaceutically acceptable carrier, excipient or buffer formulation, ranging from about 25 microliters to about 1,000 microliters, or to about 10 milliliters, or up to 20 milliliters, including all numbers within the range, depending on the size of the area to be treated, the viral titer used, the route of administration, and the desired effect of the method. In some aspects, the volume of pharmaceutically acceptable carrier, excipient or buffer is at least about 25 μl. In some aspects, the volume is about 50 μl. In another aspect, the volume is about 75 μl. In another aspect, the volume is about 100 μl. In another aspect, the volume is about 125 μl. In another aspect, the volume is about 150 μl. In another aspect, the volume is about 175 μl. In yet another aspect, the volume is about 200 μl. In another aspect, the volume is about 225 μl. In yet another aspect, the volume is about 250 μl. In yet another aspect, the volume is about 275 μl. In yet another aspect, the volume is about 300 μl. In yet another aspect, the volume is about 325 μl. In another aspect, the volume is about 350 μl. In another aspect, the volume is about 375 μl. In another aspect, the volume is about 400 μl. In another aspect, the volume is about 450 μl. In another aspect, the volume is about 500 μl. In another aspect, the volume is about 550 μl. In another aspect, the volume is about 600 μl. In another aspect, the volume is about 650 μl. In another aspect, the volume is about 700 μl. In another aspect, the volume is between about 700 and 1000 μl.

In other aspects, volumes of about 1 μl to 150 mL may be selected, with the higher volumes being selected for adults. Typically, for newborn infants a suitable volume is about 0.5 mL to about 10 mL. For older infants, about 0.5 mL to about 15 mL may be selected. For toddlers, a volume of about 0.5 mL to about 20 mL may be selected. For children, volumes of up to about 30 mL may be selected. For pre-teens and teens, volumes up to about 50 mL may be selected. In still other aspects, a patient may receive an intrathecal administration in a volume of about 5 mL to about 15 mL are selected, or about 7.5 mL to about 10 mL. Other suitable volumes and dosages may be determined. The dosage will be adjusted to balance the therapeutic benefit against any side effects and such dosages may vary depending upon the therapeutic application for which the recombinant vector is employed.

Recombinant SMN1 genes, for example in viral vectors (e.g., packaged in an rAAV), may be delivered to host cells using suitable methods. The rAAV, preferably suspended in a physiologically compatible carrier (e.g., a pharmaceutically acceptable carrier), may be administered to a human or non-human mammalian patient. In some aspects, the composition includes a pharmaceutically acceptable carrier, diluent, excipient and/or adjuvant. Suitable carriers may be selected for the route of administration. For example, one suitable carrier includes saline, which may be formulated with a variety of buffering solutions (e.g., phosphate buffered saline). Other exemplary pharmaceutically acceptable carriers include sterile saline, lactose, sucrose, calcium phosphate, gelatin, dextran, agar, pectin, peanut oil, sesame oil, and water.

In some aspects, compositions may contain, in addition to the SMN1 rAAV, small molecule for increasing SMN function (e.g., Risdiplam or Branaplam) and/or ASO (e.g., nusinersen) and pharmaceutically acceptable carrier(s), other conventional pharmaceutical ingredients, such as preservatives, or chemical stabilizers. Suitable exemplary preservatives include chlorobutanol, potassium sorbate, sorbic acid, sulfur dioxide, propyl gallate, the parabens, ethyl vanillin, glycerin, phenol, and parachlorophenol. Suitable chemical stabilizers include gelatin and albumin.

In some aspects, compositions comprising a small molecule for increasing SMN function (e.g., Risdiplam or Branaplam), an SMN1 rAAV and/or a SMN2 ASO (e.g., nusinersen) may comprise a pharmaceutically acceptable carrier and/or be admixed with suitable excipients designed for delivery to a subject via injection, osmotic pump, intrathecal catheter, or for delivery by another device or route. In one example, a composition is formulated for intrathecal delivery. In some aspects, intrathecal delivery encompasses an injection into the spinal canal, e.g., the subarachnoid space.

Viral vectors described in this application may be used in preparing a medicament for delivering SMN1 to a subject (e.g., a human patient) in need thereof, supplying functional SMN to a subject, and/or for treating spinal muscular atrophy in combination therapies with one or more SMN2 ASOs (e.g., administered concurrently or sequentially).

In some aspects, pharmaceutical compositions comprising pharmaceutically acceptable carriers (e.g., buffers, salts, and/or other components of a pharmaceutical formulation) comprising an rAAV are selected to include one or more components that prevent rAAV from sticking to infusion tubing but does not interfere with the rAAV binding activity in vivo.

In some such aspects, ASOs (e.g., SMN2 ASO)) are formulated for delivery (e.g., for systemic administration) in amounts ranging from 5 mg to 60 mg of ASO per dose. In some such aspects, ASOs (e.g., SMN2 ASO) are formulated for delivery (e.g., for systemic administration) in amounts ranging 5 mg to 20 mg of ASO per dose. In some such aspects, ASOs (e.g., SMN2 ASO) are formulated for delivery (e.g., for systemic administration) in amounts ranging 12 mg to 50 mg of ASO per dose. In some such aspects ASOs (e.g., SMN2 ASO) are formulated for delivery (e.g., for systemic administration) in amounts ranging 12 mg to 48 mg of ASO per dose. In some such aspects, ASOs (e.g., SMN2 ASO) are formulated for delivery (e.g., for systemic administration) in amounts ranging from 12 mg to 36 mg of ASO per dose. In some such aspects, ASOs (e.g., SMN2 ASO) are formulated for delivery (e.g., for systemic administration) in amounts of 28 mg of ASO per dose. In some such aspects ASOs (e.g., SMN2 ASO) are formulated for delivery (e.g., for systemic administration) in amounts of 12 mg of ASO per dose. In some such aspects, the dose volume is 5 mL.

In some such aspects, ASOs (e.g., SMN2 ASO) (alone or with a recombinant SMN1 gene and or a small molecule for increasing SMN function such as Risdiplam or Branaplam) are formulated for delivery (e.g., for systemic administration) ranging from 0.1 mg/kg to 200 mg/kg (ASO/patient weight). In some aspects, the dose is from 0.1 mg/kg to 100 mg/kg. In some aspects, the dose is from 0.5 mg/kg to 100 mg/kg. In some aspects, the dose is from 1 mg/kg to 100 mg/kg. In some aspects, the dose is from 1 mg/kg to 50 mg/kg. In some aspects, the dose is from 1 mg/kg to 25 mg/kg. In some aspects, the dose is from 0.1 mg/kg to 25 mg/kg. In some aspects, the dose is from 0.1 mg/kg to 10 mg/kg. In some aspects, the dose is from 1 mg/kg to 10 mg/kg. In some aspects, the dose is from 1 mg/kg to 5 mg/kg. In some aspects, dosing a subject with an ASO is divided into an induction phase and a maintenance phase. In some such aspects, the dose administered during the induction phase is greater than the dose administered during the maintenance phase. In some aspects, the dose administered during the induction phase is less than the dose administered during the maintenance phase. In some aspects, the induction phase is achieved by bolus injection and the maintenance phase is achieved by continuous infusion. In some aspects, a combination formulation is used during the induction phase.

In some aspects, pharmaceutical compositions are administered as a bolus injection. In some such aspects, the dose of the bolus injection contains a total of 5 mg to 60 mg per dose of an antisense oligonucleotide (e.g., SMN2 ASO). In some such aspects, the dose of the bolus injection contains a total of 5 mg to 20 mg per dose of an antisense oligonucleotide (e.g., SMN2 ASO). In some such aspects, the dose of the bolus injection contains a total of 12 mg to 50 mg per dose of an antisense oligonucleotide (e.g., SMN2 ASO). In some such aspects, the dose of the bolus injection contains a total of 12 mg to 48 mg per dose of an antisense oligonucleotide (e.g., SMN2 ASO). In some such aspects, the dose of the bolus injection contains a total of 12 mg to 36 mg per dose of an antisense oligonucleotide (e.g., SMN2 ASO). In some such aspects, the dose of the bolus injection contains a total of 28 mg per dose of an antisense oligonucleotide (e.g., SMN2 ASO). In some such aspects, the dose of the bolus injection contains a total of 12 mg per dose of an antisense oligonucleotide (e.g., SMN2 ASO). In some such aspects, the dose volume is 5 mL.

In some aspects, pharmaceutical compositions are administered as a bolus injection. In some such aspects, the dose of the bolus injection is from 0.01 to 25 milligrams of antisense compound per kilogram body weight of the subject. In some such aspects, the dose of the bolus injection is from 0.01 to 10 milligrams of antisense compound per kilogram body weight of the subject. In some aspects, the dose is from 0.05 to 5 milligrams of antisense compound per kilogram body weight of the subject. In some aspects, the dose is from 0.1 to 2 milligrams of antisense compound per kilogram body weight of the subject. In some aspects, the dose is from 0.5 to 1 milligrams of antisense compound per kilogram body weight of the subject.

In some aspects, such doses are administered twice monthly. In some aspects, such doses are administered every month. In some aspects, such doses are administered every 2 months. In some aspects, such doses are administered every 6 months. In some aspects, such doses are administered by bolus injection into the CSF. In some aspects, such doses are administered by intrathecal bolus injection. In some aspects, such doses are administered by bolus systemic injection (e.g., subcutaneous, intramuscular, or intravenous injection). In some aspects, subjects receive bolus injections into the CSF and bolus systemic injections. In such aspects, the doses of the CSF bolus and the systemic bolus may be the same or different from one another. In some aspects, the CSF and systemic doses are administered at different frequencies. In some aspects, the invention provides a dosing regimen comprising at least one bolus intrathecal injection and at least one bolus subcutaneous injection.

In some aspects, pharmaceutical compositions are administered by continuous infusion (e.g., wherein a dose can be administered over a period time, for example, a 24 hour period). Such continuous infusion may be accomplished by an infusion pump that delivers pharmaceutical compositions to the CSF. In some aspects, such infusion pump delivers pharmaceutical composition IT or ICV. In some such aspects, the dose administered is between 5 mg to 60 mg per dose of an antisense oligonucleotide (e.g., SMN2 ASO) per day. In some such aspects, the dose administered is between 5 mg to 20 mg per dose of an antisense oligonucleotide (e.g., SMN2 ASO) per day. In some such aspects, the dose administered is between 12 mg to 50 mg per dose of an antisense oligonucleotide (e.g., SMN2 ASO) per day. In some such aspects, the dose administered is between 12 mg to 48 mg per dose of an antisense oligonucleotide (e.g., SMN2 ASO) per day. In some such aspects, the dose administered is between 12 mg to 36 mg per dose of an antisense oligonucleotide (e.g., SMN2 ASO) per day. In some such aspects, the dose administered is 28 mg per dose of an antisense oligonucleotide (e.g., SMN2 ASO) per day. In some such aspects, the dose administered is 12 mg per dose of an antisense oligonucleotide (e.g., SMN2 ASO) per day. In some such aspects, the dose volume is 5 mL.

In some aspects, the dose administered is between 0.05 and 25 milligrams of antisense compound per kilogram body weight of the subject per day. In some aspects, the dose administered is from 0.1 to 10 milligrams of antisense compound per kilogram body weight of the subject per day. In some aspects, the dose administered is from 0.5 to 10 milligrams of antisense compound per kilogram body weight of the subject per day. In some aspects, the dose administered is from 0.5 to 5 milligrams of antisense compound per kilogram body weight of the subject per day. In some aspects, the dose administered is from 1 to 5 milligrams of antisense compound per kilogram body weight of the subject per day.

In some aspects, the invention provides a dosing regimen comprising infusion into the CNS and at least one bolus systemic injection. In some aspects, the invention provides a dosing regimen comprising infusion into the CNS and at least one bolus subcutaneous injection. In some aspects, the dose, whether by bolus or infusion, is adjusted to achieve or maintain a concentration of antisense compound from 0.1 to 100 microgram per gram of CNS tissue. In some aspects, the dose, whether by bolus or infusion, is adjusted to achieve or maintain a concentration of antisense compound from 1 to 10 microgram per gram of CNS tissue. In some aspects, the dose, whether by bolus or infusion, is adjusted to achieve or maintain a concentration of antisense compound from 0.1 to 1 microgram per gram of CNS tissue.

Accordingly, in some aspects, the present invention provides pharmaceutical compositions comprising one or more therapeutic molecules, for example one or more recombinant nucleic acids (e.g., in a viral vector, for example packaged in an rAAV) and/or antisense compounds. In some aspects, such pharmaceutical composition comprises a sterile saline solution and one or more therapeutic molecules. In some aspects, such pharmaceutical compositions consist of a sterile saline solution and one or more therapeutic molecules. In some aspects, therapeutic molecules may be admixed with pharmaceutically acceptable active and/or inert substances for the preparation of pharmaceutical compositions or formulations. Compositions and methods for the formulation of pharmaceutical compositions depend on a number of criteria, including, but not limited to, route of administration, extent of disease, or dose to be administered. In some aspects, therapeutic molecules can be utilized in pharmaceutical compositions by combining such therapeutic molecules with a suitable pharmaceutically acceptable diluent or carrier. In some aspects, a pharmaceutically acceptable diluent includes phosphate-buffered saline (PBS). PBS is a diluent suitable for use in compositions to be delivered parenterally. Accordingly, in some aspects, employed in the methods described herein is a pharmaceutical composition comprising one or more therapeutic molecules and a pharmaceutically acceptable diluent. In some aspects, the pharmaceutically acceptable diluent is PBS. Pharmaceutical compositions comprising one or more therapeutic molecules described in this application encompass any pharmaceutically acceptable salts, esters, or salts of such esters. In some aspects, pharmaceutical compositions comprising ASOs comprise one or more oligonucleotide which, upon administration to an animal, including a human, is capable of providing (directly or indirectly) the biologically active metabolite or residue thereof. Accordingly, in some aspects pharmaceutically acceptable salts of ASOs, prodrugs, pharmaceutically acceptable salts of such prodrugs, and other bioequivalents are provided. Suitable pharmaceutically acceptable salts include, but are not limited to, sodium and potassium salts.

In some aspects, a prodrug can include the incorporation of additional nucleosides at one or both ends of an oligomeric compound which are cleaved by endogenous nucleases within the body, to form the active antisense oligomeric compound. Lipid-based vectors have been used in nucleic acid therapies in a variety of methods. For example, in one method, the nucleic acid is introduced into preformed liposomes or lipoplexes made of mixtures of cationic lipids and neutral lipids. In another method, DNA complexes with mono- or poly-cationic lipids are formed without the presence of a neutral lipid. Some preparations are described in Akinc et al., Nature Biotechnology 26, 561-569 (1 May 2008), which is herein incorporated by reference in its entirety.

### Kits

In some aspects, kits are provided comprising a small molecule for increasing SMN function, a recombinant SMN1 gene (e.g., in an rAAV) and/or a SMN2 ASO, e.g., in a pharmaceutical composition. In some aspects, such kits further comprise additional therapeutic agents such as one or more immunosuppressive agents. In some aspects, such kits further comprise a means of delivery, for example a syringe or infusion pump.

The following examples are illustrative only and are not intended to limit the present invention.

### EXAMPLES

### Example 1: rAAV Vectors Containing an hSMN1 Gene

A recombinant neurotropic AAV virus was constructed bearing a codon-optimized human SMN1 cDNA.

### Example 2: ASOs that Increases Full-Length SMN2 mRNA (e.g., by Promoting Exon 7 Inclusion in hSMN2 mRNA)

An ASO that increases full-length SMN2 mRNA (e.g., that promotes exon 7 inclusion in SMN2 mRNA) was prepared (FIG. 3).

### Example 3: Administration and Bio-Distribution of rAAV Vectors Containing an hSMN1 Gene with an ASO that Increases Full-Length SMN2 mRNA (e.g., that Promotes Exon 7 Inclusion in Smn2 mRNA)

The rAAV of Example 1 and the ASO of Example 2 are administered to animal SMA disease models and control animals, including mice, pig, and non-human primate (e.g., macaque), SMA disease and control animal models.

The rAAV and ASO are administered via different routes, including via intrathecal and systemic routes (e.g., via lumbar puncture, intra-cisterna magna, and intravenous delivery).

The distribution of rAAV and ASO is evaluated in the animal models. In particular, distribution within the spinal cord is evaluated, for example to determine the relative amount of rAAV and/or ASO in the cervical, thoracic, and lumbar regions of the spinal cord.

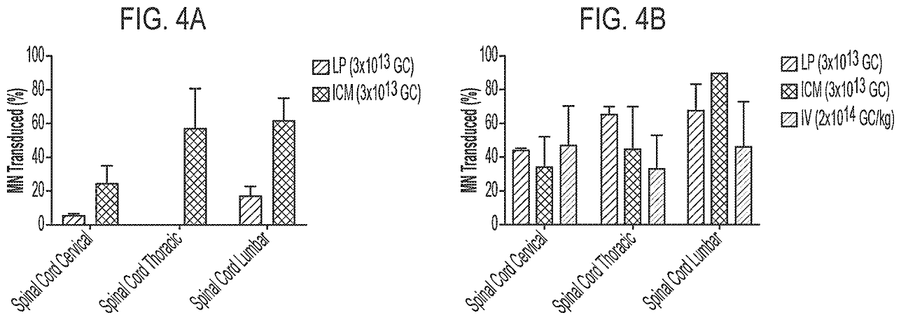

FIG. 4 illustrates results using 3×10¹³ GC rAAV administered via lumbar puncture or intra-cisterna magna delivery, and using 2×10¹⁴ GC administered intravenously.

### Example 4: Co-Formulation of rAAV Vectors Containing an hSMN1 Gene with an ASO that Increases Full-Length SMN2 mRNA (e.g., that Promotes Exon 7 Inclusion in SMN2 mRNA)

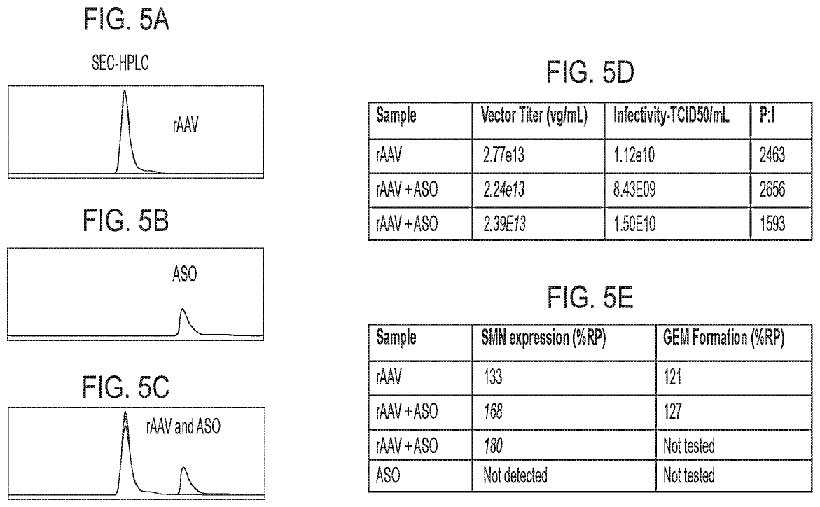

FIG. 5 illustrates non-limiting examples of physical and biological characterizations of a composition comprising both an rAAV vector an hSMN1 gene and an ASO that increases full-length SMN2 mRNA (e.g., that promotes exon 7 inclusion in SMN2 mRNA).

FIG. 5A shows an SEC-HPLC profile of the rAAV vector alone. FIG. 5B shows an SEC-HPLC profile of the ASO alone. FIG. 5C shows an SEC-HPLC profile of the rAAV vector and the ASO when they are present in the same formulation. The HPLC profiles of the rAAV vector and ASO remain the same in FIG. 5C, showing that there is no significant incompatibility when the rAAV and the ASO are co-formulated.

FIG. 5D provides data for rAAV infectivity in cells in vitro upon delivery of either the rAAV vector alone or with the ASO. The results show that rAAV infectivity is not significantly affected by the presence of the ASO in a co-formulation.

FIG. 5E shows intracellular SMN protein expression level and GEM formation in cells following treatment with rAAV, ASO, or both.

### Example 5: Intracerebroventricular (ICV) Administration of Nusinersen and AAV-SMN1

Using a micro-osmotic pump (ALZET Osmotic Pumps, Cupertino, Calif., USA), Nusinersen and AAV-SMN1 are delivered into cerebrospinal fluid (CSF) through the right lateral ventricle in neonatal (P0-P1) SMA mice with a human SMN2 transgene. A low or high dose of nusinersen (1 μg and 4 μg respectively) is administered to the mice along with a low or high dose of AAV-SMN1 (1×10¹⁰ GC or 8×10¹⁰ GC respectively) at birth (P0-P1).

The mice body weight and righting reflex is measured and compared to the body weight and righting reflex of control mice of the same genotype having received either nusinersen or AAV-SMN1 alone.

Mice administered both nusinersen and AAV-SMN1 will have a significantly higher body weight and faster righting reflex compared to controls.

Studies will reveal that intracerebroventricular (ICV) administration of nusinersen and AAV-SMN1 increases SMN2 exon 7 inclusion in the spinal cord. Further studies will show that a greater number of spinal-cord motor neurons have increased SMN expression compared to controls.

### Example 6: Administration of Compositions of Nusinersen and AAV-SMN1

Using a micro-osmotic pump (ALZET Osmotic Pumps, Cupertino, Calif., USA), compositions of nusinersen and AAV-SMN1 are delivered into cerebrospinal fluid (CSF) through the right lateral ventricle in neonatal (P0-P1) SMA mice with a human SMN2 transgene. Compositions of a low dose of nusinersen (1 μg) and a low dose of AAV-SMN1 (1×10¹⁰ GC), or a low dose of nusinersen (1 μg) and a high dose of AAV-SMN1 (8×10¹⁰ GC), or a high dose of nusinersen (4 μg) and a low dose of AAV-SMN1 (1×10¹⁰ GC), or a high dose of nusinersen (4 μg) and a high dose of AAV-SMN1 (8×10¹⁰ GC) are administered to the mice at birth (P0-P1). The mice body weight and righting reflex is measured and compared to the body weight and righting reflex of control mice of the same genotype having received either nusinersen or AAV-SMN1 alone.

Mice administered a composition of nusinersen and AAV-SMN1 will have a significantly higher body weight and faster righting reflex compared to controls.

Studies will reveal that intracerebroventricular (ICV) administration of the composition of nusinersen and AAV-SMN1 increases SMN2 exon 7 inclusion in the spinal cord. Further studies will show that a greater number of spinal-cord motor neurons have increased SMN expression compared to controls.

### Example 7: Administration and Analysis of Nusinersen and AAV-SMN1 Distribution in Non-Human Mammals

SMA mice, Rhesus Macaques and Cynomolgus monkeys are used to assess distribution of nusinersen and AAV-SMN1 compositions at different doses and routes of administration. Nusinersen and AAV-SMN1 compositions are administered to some mice and some monkeys at a dose of about 1 mg/kg by intracerebroventricular (ICV) infusion or by intrathecal (IT) infusion over a 24 hour period. The animals are sacrificed and tissues harvested 96 hours after the end of the infusion period. The concentration of nusinersen and AAV-SMN1 are measured in samples from Cervical, Thoracic, and Lumbar sections of the spinal cord.

Additional mice, Rhesus Macaques and Cynomolgus monkeys of the same genotype as above, are administered nusinersen and AAV-SMN1 compositions at the same dose of about 1 mg/kg by ICV infusion or by IT infusion. The animals are administered the nusinersen and AAV-SMN1 compositions over a period 3 days, 7 days, or 14 days prior to being sacrificed 5 days after the end of the infusion period.

### Example 8: Administration of Nusinersen and AAV-SMN1 to Human Subjects

Nusinersen and AAV-SMN1 are administered to human subjects using routes comprising intracerebroventricular (ICV), intravenous (IV), and intrathecal (IT) (e.g., via lumbar puncture (LP), and/or intracisternal magna (ICM) delivery). The compositions are tested in both children and adults.

In some aspects, rAAV-SMN1 compositions are administered to children (e.g., having SMA) at a dose of about 1×10¹⁴ GC, for example by lumbar puncture (LP) infusion (e.g., over a 24 hour period). In some aspects, rAAV-SMN1 compositions are administered to adults (e.g., having SMA) at a dose of about 1.5×10¹⁴ GC, for example by intracisternal magna (ICM) infusion (e.g., over a 24 hour period).

In some aspects, other rAAV-SMN1 doses can be used, for example about 5-6×10¹³ GC, or higher, for example, around 1.2×10¹⁴ GC, or 1.5-1.8×10¹⁴ GC. Any suitable route of administration can be used, for example via IT delivery (e.g., infusion over a 24 hour period), for example via LP or ICM delivery.

### Example 9: Intracerebroventricular (ICV) Administration of Nusinersen and AAV-SMN1

Nusinersen and AAV-SMN1 were administered to neonatal (P0-P1) SMA mice having a human SMN2 transgene. A low or high dose of nusinersen (1 μg and 3 μg respectively) was administered to the mice along with a low or high dose of AAV-SMN1 (1×10¹⁰ GC or 3×10¹⁰ GC respectively) at birth (P0-P1). The mice body weight and righting reflex were measured and compared to the body weight and righting reflex of control mice of the same genotype having received either nusinersen or AAV-SMN1 alone.

Mice administered both nusinersen and AAV-SMN1 have a significantly higher body weight and faster righting reflex compared to controls.

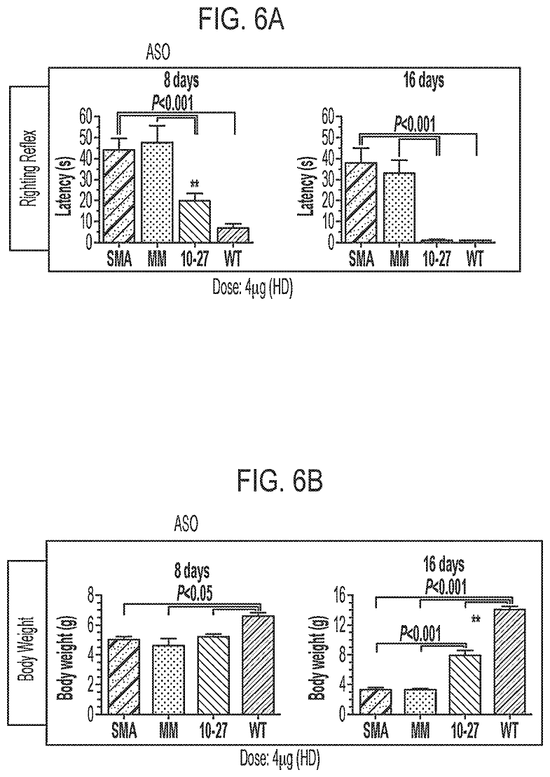

FIGS. 6A-6B either an SMN1 gene (e.g., in an rAAV vector) or an ASO such as nusinersen (e.g., in a single dose). The experiments show partial rescue of motor function at postnatal day (PND) 8** with full rescue at PND 16, post dosing. FIG. 6A shows the righting reflex (RR) of 4 separate groups of mice after 8 and 16 days of nusinersen. FIG. 6B shows the body weight of 4 separate groups of mice after 8 and 16 days of nusinersen. A combination therapy can improve on the partial rescue of RR (PND 7-16) and body weight seen with monotherapy.

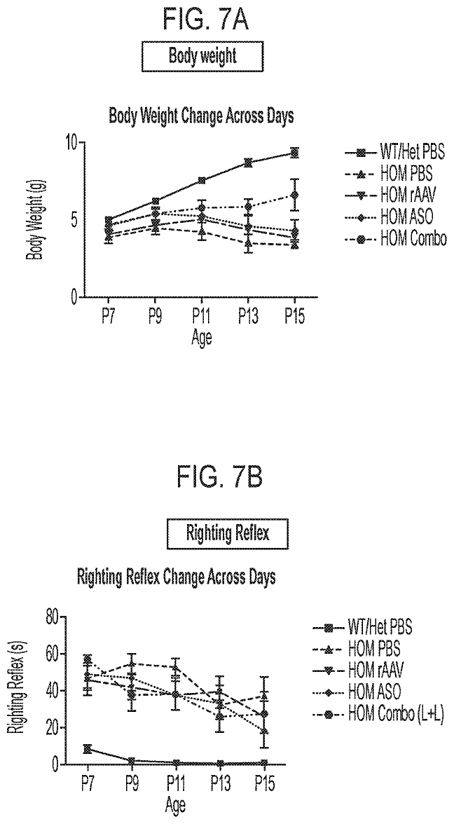

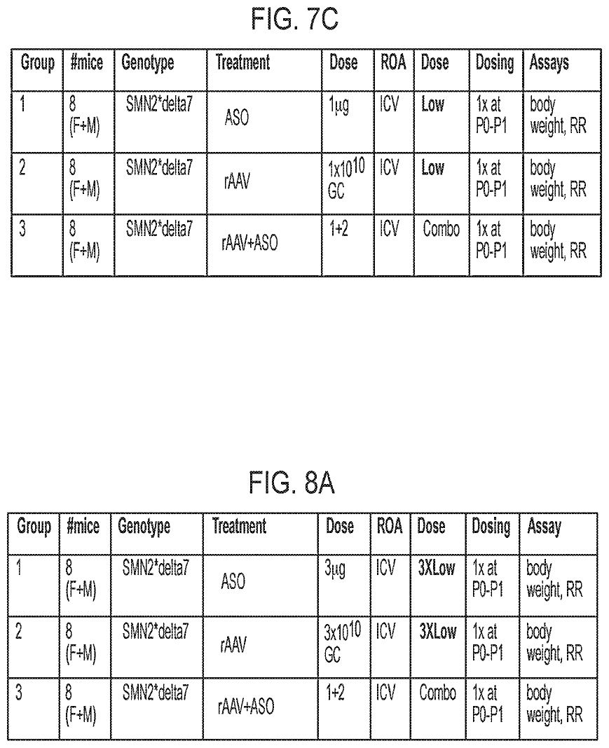

FIGS. 7A-7C show the results of a first combination therapy study showing the effect of SMN1 gene therapy with nusinersen on body weight and RR. FIG. 7A shows body weight change over time. FIG. 7B shows RR change over time. FIG. 7C is a chart outlining conditions for the three groups of animals that were tested.

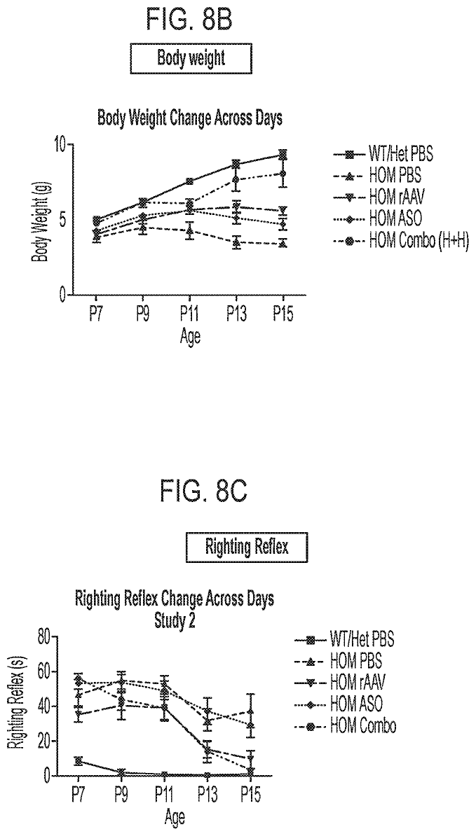

FIGS. 8A-8C show the results of a second combination therapy showing the effect of a SMN1 gene therapy with nusinersen on body weight and RR. FIG. 8A is a chart outlining conditions for the three groups of animals that were tested. FIG. 8B shows the body weight change over time, and FIG. 8C shows the RR change over time (in days).

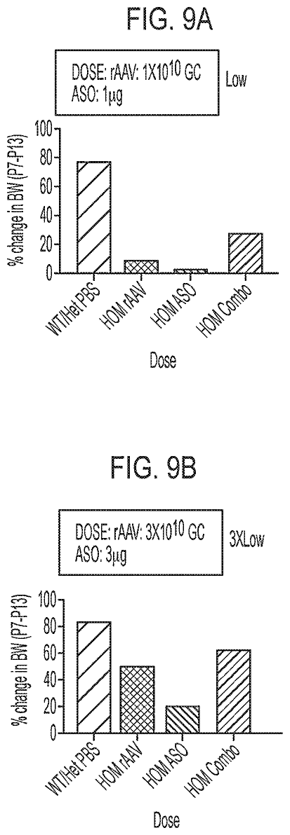

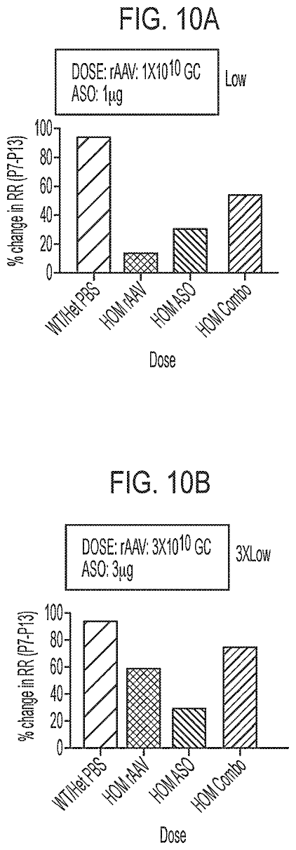

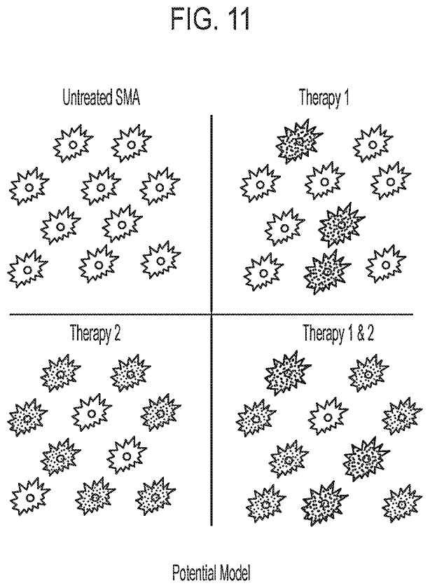

FIGS. 9A-9B show the comparison of % change in body weight from PND 7-PND 13. FIG. 9A shows the % change in body weight at a dose of gene therapy (rAAV): 1×10¹⁰ GC/ASO (nusinersen): 1 μg. FIG. 9B shows the % change in body weight a dose of gene therapy (rAAV): 3×10¹⁰ GC/ASO (nusinersen): 3 μg. FIGS. 10A-10B show the comparison of % change in RR from PND 7-PND 13. FIG. 10A shows the % change in RR at a dose of gene therapy (rAAV): 1×10¹⁰ GC/ASO (nusinersen): 1 μg. FIG. 10B shows the % change in RR at a dose of gene therapy (rAAV): 3×10¹⁰ GC/ASO (nusinersen): 3 μg.

### OTHER ASPECTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

From the above description, one skilled in the art can easily ascertain the essential characteristics of the present disclosure, and without departing from the spirit and scope thereof, can make various changes and modifications of the disclosure to adapt it to various usages and conditions. Thus, other aspects are also within the claims.

### EQUIVALENTS

While several inventive aspects have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive aspects described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive aspects described herein. It is, therefore, to be understood that the foregoing aspects are presented by way of example only and that, within the scope of the appended claims and equivalents thereto, inventive aspects may be practiced otherwise than as specifically described and claimed. Inventive aspects of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one aspect, to A only (optionally including elements other than B); in another aspect, to B only (optionally including elements other than A); in yet another aspect, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one aspect, to at least one, optionally including more than one, A, with no B present (and optionally including elements other than B); in another aspect, to at least one, optionally including more than one, B, with no A present (and optionally including elements other than A); in yet another aspect, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

In the claims, as well as in the specification above, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively, as set forth in the United States Patent Office Manual of Patent Examining Procedures, Section 2111.03. It should be appreciated that aspects described in this document using an open-ended transitional phrase (e.g., “comprising”) are also contemplated, in alternative aspects, as “consisting of” and “consisting essentially of” the feature described by the open-ended transitional phrase. For example, if the disclosure describes “a composition comprising A and B”, the disclosure also contemplates the alternative aspects “a composition consisting of A and B” and “a composition consisting essentially of A and B”.

Although the sequence listing accompanying this filing identifies each sequence as either “RNA” or “DNA” as required, in reality, those sequences may be modified with any combination of chemical modifications. One of skill in the art will readily appreciate that such designation as “RNA” or “DNA” to describe modified oligonucleotides is, in some instances, arbitrary. For example, an oligonucleotide comprising a nucleoside comprising a 2′-OH Sugar moiety and a thymine base could be described as a DNA having a modified sugar (2′-OH for the natural 2′-H of DNA) or as an RNA having a modified base (thymine(methylated uracil) for natural uracil of RNA).

Accordingly, nucleic acid sequences provided herein, including, but not limited to those in the sequence listing, are intended to encompass nucleic acids containing any combination of natural or modified RNA and/or DNA, including, but not limited to such nucleic acids having modified nucleobases. By way of further example and without limitation, an oligomeric compound having the nucleobase sequence “ATCGATCG” encompasses any oligomeric compounds having such nucleobase sequence, whether modified or unmodified, including, but not limited to such compounds comprising RNA bases, such as those having sequence “AUCGAUCG” and those having some DNA bases and some RNA bases such as “AUCGATCG” and oligomeric compounds having other modified bases such as “AT″CGAUCG,” wherein ″C indicates a cytosine base comprising a methyl group at the 5-position.

What is claimed is:

1. A method of treating spinal muscular atrophy (SMA) in a subject having SMA, the method comprising administering to the subject:
a) a small molecule that increases SMN function, and
b) a recombinant nucleic acid that encodes the survival of motor neuron 1 (SMN1) protein.

2. A method of treating spinal muscular atrophy (SMA) in a subject having SMA, the method comprising administering to the subject:
a) a small molecule that increases SMN function, and
b) an antisense oligonucleotide (ASO) that increases full-length survival of motor neuron 2 (SMN2) mRNA.

3. A method of treating spinal muscular atrophy (SMA) in a subject having SMA, the method comprising administering to the subject:
a) a small molecule that increases SMN function,
b) a recombinant nucleic acid that encodes the survival of motor neuron 1 (SMN1) protein, and
c) an antisense oligonucleotide (ASO) that increases full-length survival of motor neuron 2 (SMN2) mRNA.

4. The method of any one of claims 1-3, wherein the subject has a deletion or mutation in each survival motor neuron 1 (SMN1) allele.

5. The method of claim 4, wherein the subject is homozygous for a SMN1 gene mutation.

6. The method of any one of claims 1-5, wherein the subject has one or more symptoms of SMA.

7. The method of claim 6, wherein the symptoms comprise atrophy of the limb muscles, difficulty or inability walking, or difficulty breathing.

8. The method of any one of claims 1-7, wherein the subject is a human subject selected from the pediatric and adult population.

9. The method of any one of claims 1-8, wherein the small molecule that increases SMN function is a substituted pyridazine.

10. The method of claim 9, wherein the small molecule drug is a substituted pyridazine of Formula (I′): 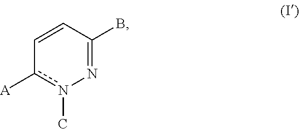
or a pharmaceutically acceptable salt thereof, wherein:
A is 2-hydroxy-phenyl which is substituted with 0, 1, 2, or 3 substituents independently selected from C₁-C₄alkyl, wherein 2 C₁-C₄alkyl groups can combine with the atoms to which they are bound to form a 5 to 6 membered ring and is substituted with 0 or 1 substituents selected from oxo, oxime and hydroxy, haloC₁-C₄alkyl, dihaloC₁-C₄alkyl, trihaloC₁-C₄alkyl, C₁-C₄alkoxy, C₁-C₄alkoxy-, C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH, C₁-C₄alkyl, -heteroaryl, —NHC(O)—, C₁-C₄alkyl-, heteroaryl, C₁-C₄alkyl-C(O)NH—, heteroaryl, C₁-C₄alkyl NHC(O)-heteroaryl, 3-7 membered cycloalkyl, 5-7 membered cycloalkenyl or 5, 6, or 9 membered heterocycle containing 1 or 2 heteroatoms, independently, selected from S, O and N, wherein heteroaryl has 5, 6, or 9 ring atoms, 1, 2, or 3 ring heteroatoms selected from N, O and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7member heterocycleC₁-C₄alkyl, aminoC₁-C₄alkyl and mono- and di-C₁-C₄alkylaminoC1-C₄alkyl; or
A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₅alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, alkylene 4 to 7 member heterocycle, 4 to 7 member heterocycle and mono- and di-C₁-C₄alkylamino; or
A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O, and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC1-C₄alkyl; or
A is bicyclic heteroaryl having 9 to 10 ring atoms and 1, 2, or 3 ring heteroatoms independently selected from N, O, or S, which bicyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino and mono- and di-C₁-C₄alkylamino; or
A is tricyclic heteroaryl having 12 or 13 ring atoms and 1, 2, or 3 ring heteroatoms independently selected from N, O, or S, which tricyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy, C₁-C₄alkoxy substituted with hydroxy, C₁-C₄alkoxy, amino, mono- and di-C₁-C₄alkylamino and heteroaryl, wherein said heteroaryl has 5, 6, or 9 ring atoms, 1, 2, or 3 ring heteroatoms selected from N, O, and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC1-C₄alkyl;
B is a group of the formula: 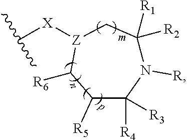
wherein:
m, n and p are independently selected from 0 or 1;
R, R₁, R₂, R₃, and R₄ are independently selected from the group consisting of hydrogen, C₁-C₄alkyl, which alkyl is optionally substituted with hydroxy, amino, or mono- and di-C₁-C₄alkylamino;
R₅ and R₆ are independently selected from hydrogen and fluorine; or
R and R₃, taken in combination form a fused 5 or 6 member heterocyclic ring having 0 or 1 additional ring heteroatoms selected from N, O, or S;
R₁ and R₃, taken in combination form a C₁-C₃alkylene group;
R₁ and R₅, taken in combination form a C₁-C₃alkylene group;
R₃ and R₄, taken in combination with the carbon atom to which they attach, form a spirocyclicC₃-C₆cycloalkyl;
X is CR_{A}R_{B}, O, NR₇, or a bond;
R₇ is hydrogen or C₁-C₄alkyl;
R_{A} and R_{B} are independently selected from hydrogen and C₁-C₄alkyl, or R_{A} and R_{B}, taken in combination, form a divalent C₂-C₅alkylene group;
Z is CR₈ or N; when Z is N, X is a bond;
R₈ is hydrogen or taken in combination with R₆ form a double bond; or
B is a group of the formula: 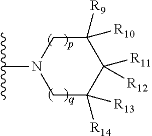
wherein:
p and q are independently selected from the group consisting of 0, 1, and 2;
R₉ and R₁₃ are independently selected from hydrogen and C₁-C₄alkyl;
R₁₀ and R₁₄ are independently selected from hydrogen, amino, mono- and di-C₁-C₄alkylamino, and C₁-C₄alkyl, which alkyl is optionally substituted with hydroxy, amino or mono- and di-C₁-C₄alkylamino;
R₁₁ is hydrogen, C₁-C₄alkyl, amino, or mono- and di-C₁-C₄alkylamino;
R₁₂ is hydrogen or C₁-C₄alkyl; or
R₉ and R₁₀, taken in combination form a saturated azacycle having 4 to 7 ring atoms, which is optionally substituted with 1-3 C₁-C₄alkyl groups; or
R₁₁ and R₁₂, taken in combination form a saturated azacycle having 4 to 7 ring atoms which is optionally substituted with 1-3 C₁-C₄alkyl groups; and
C is H or absent, as valency permits.

11. The method of claim 10, wherein A is 2-hydroxy-phenyl which is substituted with 0, 1, 2, or 3 substituents independently selected from C₁-C₄alkyl, wherein 2 C₁-C₄alkyl groups can combine with the atoms to which they are bound to form a 5 to 6 membered ring and is substituted with 0 or 1 substituents selected from oxo, oxime and hydroxy, haloC₁-C₄alkyl, dihaloC₁-C₄alkyl, trihaloC₁-C₄alkyl, C₁-C₄alkoxy, C₁-C₄alkoxy-, C₃-C₇cycloalkyl, haloC₁-C₄alkoxy, dihaloC₁-C₄alkoxy, trihaloC₁-C₄alkoxy, hydroxy, cyano, halogen, amino, mono- and di-C₁-C₄alkylamino, heteroaryl, C₁-C₄alkyl substituted with hydroxy, C₁-C₄alkoxy substituted with aryl, amino, —C(O)NH, C₁-C₄alkyl, -heteroaryl, —NHC(O)—, C₁-C₄alkyl-, heteroaryl, C₁-C₄alkyl-C(O)NH—, heteroaryl, C₁-C₄alkyl NHC(O)-heteroaryl, 3-7 membered cycloalkyl, 5-7 membered cycloalkenyl or 5, 6, or 9 membered heterocycle containing 1 or 2 heteroatoms, independently, selected from S, O, and N, wherein heteroaryl has 5, 6, or 9 ring atoms, 1, 2, or 3 ring heteroatoms selected from N, O, and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl and mono- and di-C₁-C₄alkylaminoC1-C₄alkyl.

12. The method of claims 10 or 11, wherein A is of the formula: 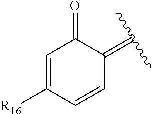
wherein R₁₆ is a 5 member heteroaryl having one ring nitrogen atom and 0 or 1 additional ring heteroatom selected from N, O, or S, wherein the heteroaryl is optionally substituted with C₁-C₄alkyl.

13. The method of any one of claims 11 or 12, wherein A is of the formula:

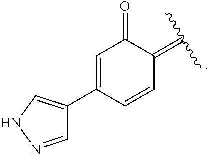 [SMILES: *=C1C=CC(c2cn[nH]c2)=CC1=O]

14. The method of claim 10, wherein A is bicyclic heteroaryl having 9 to 10 ring atoms and 1, 2, or 3 ring heteroatoms independently selected from N, O, or S, which bicyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy and C₁-C₄alkoxy substituted with hydroxy, C₁-C₄alkoxy, amino, and mono- and di-C₁-C₄alkylamino.

15. The method of claim 14, wherein A is 2-naphthyl optionally substituted at the 3 position with hydroxy and additionally substituted with 0, 1, or 2 substituents selected from hydroxy, cyano, halogen, C₁-C₄alkyl, C₂-C₄alkenyl, C₁-C₅alkoxy, wherein the alkoxy is unsubstituted or substituted with hydroxy, C₁-C₄alkoxy, amino, N(H)C(O)C₁-C₄alkyl, N(H)C(O)₂ C₁-C₄alkyl, alkylene 4 to 7 member heterocycle, 4 to 7 member heterocycle, and mono- and di-C₁-C₄alkylamino.

16. The method of claim 10, wherein A is 6 member heteroaryl having 1-3 ring nitrogen atoms, which 6 member heteroaryl is substituted by phenyl or a heteroaryl having 5 or 6 ring atoms, 1 or 2 ring heteroatoms independently selected from N, O, and S and substituted with 0, 1, or 2 substituents independently selected from C₁-C₄alkyl, mono- and di-C₁-C₄alkylamino, hydroxyC₁-C₄alkylamino, hydroxyC₁-C₄alkyl, aminoC₁-C₄alkyl and mono- and di-C₁-C₄alkylaminoC₁-C₄alkyl.

17. The method of claim 10, wherein A is tricyclic heteroaryl having 12 or 13 ring atoms and 1, 2, or 3 ring heteroatoms independently selected from N, O, or S, which tricyclic heteroaryl is substituted with 0, 1, or 2 substituents independently selected from cyano, halogen, hydroxy, C₁-C₄alkyl, C₂-C₄alkenyl, C₂-C₄alkynyl, C₁-C₄alkoxy, C₁-C₄alkoxy substituted with hydroxy, C₁-C₄alkoxy, amino, mono- and di-C₁-C₄alkylamino and heteroaryl, wherein said heteroaryl has 5, 6, or 9 ring atoms, 1, 2, or 3 ring heteroatoms selected from N, O, and S and substituted with 0, 1, or 2 substituents independently selected from oxo, hydroxy, nitro, halogen, C₁-C₄alkyl, C₁-C₄alkenyl, C₁-C₄alkoxy, C₃-C₇cycloalkyl, C₁-C₄alkyl-OH, trihaloC₁-C₄alkyl, mono- and di-C₁-C₄alkylamino, —C(O)NH₂, —NH₂, —NO₂, hydroxyC₁-C₄alkylamino, hydroxyC₁-C₄alkyl, 4-7 member heterocycleC₁-C₄alkyl, aminoC₁-C₄alkyl, and mono- and di-C₁-C₄alkylaminoC₁-C₄alkyl.

18. The method of any one of claims 10-17, wherein B is of the formula: 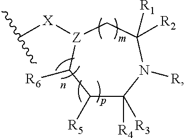
wherein:
m, n, and p are independently selected from 0 or 1;
R, R₁, R₂, R₃, and R₄ are independently selected from the group consisting of hydrogen, C₁-C₄alkyl, which alkyl is optionally substituted with hydroxy, amino, or mono- and di-C₁-C₄alkylamino;
R₅ and R₆ are independently selected from hydrogen and fluorine; or
R and R₃, taken in combination form a fused 5 or 6 member heterocyclic ring having 0 or 1 additional ring heteroatoms selected from N, O, or S;
R₁ and R₃, taken in combination form a C₁-C₃alkylene group;
R₁ and R₅, taken in combination form a C₁-C₃alkylene group;
R₃ and R₄, taken in combination with the carbon atom to which they attach, form a spirocyclicC₃-C₆cycloalkyl;
X is CR_{A}R_{B}, O, NR₇, or a bond;
R₇ is hydrogen or C₁-C₄alkyl;
R_{A} and R_{B} are independently selected from hydrogen and C₁-C₄alkyl, or R_{A} and R_{B}, taken in combination, form a divalent C₂-C₅alkylene group;
Z is CR₈ or N; when Z is N, X is a bond;
R₈ is hydrogen or taken in combination with R₆ form a double bond.

19. The method of any one of claims 10-17, wherein B is of the formula: 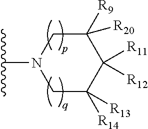
wherein:
p and q are independently selected from the group consisting of 0, 1, and 2;
R₉ and R₁₃ are independently selected from hydrogen and C₁-C₄alkyl;
R₁₀ and R₁₄ are independently selected from hydrogen, amino, mono- and di-C₁-C₄alkylamino and C₁-C₄alkyl, which alkyl is optionally substituted with hydroxy, amino, or mono- and di-C₁-C₄alkylamino;
R₁₁ is hydrogen, C₁-C₄alkyl, amino, or mono- or di-C₁-C₄alkylamino;
R₁₂ is hydrogen or C₁-C₄alkyl; or
R₉ and R₁₀, taken in combination form a saturated azacycle having 4 to 7 ring atoms, which is optionally substituted with 1-3 C₁-C₄alkyl groups; or
R₁₁ and R₁₂, taken in combination form a saturated azacycle having 4 to 7 ring atoms which is optionally substituted with 1-3 C₁-C₄alkyl groups.

20. The method of claim 19, wherein B is of the formula: 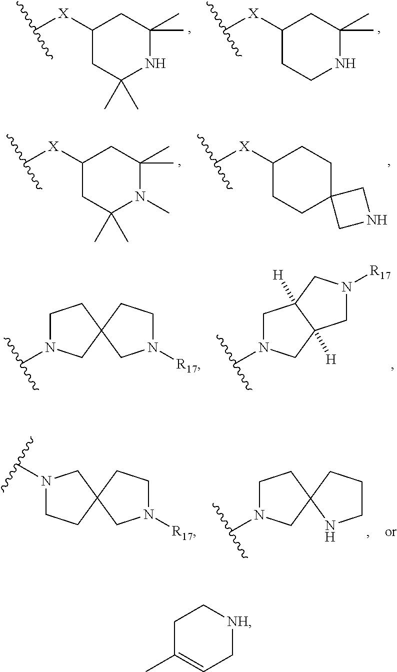
wherein R₁₇ is H or unsubstituted methyl.

21. The method of claim 19 or 20, wherein B is of the formula

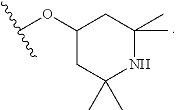 [SMILES: *OC1CC(C)(C)NC(C)(C)C1]

22. The method of any one of claims 10-13, wherein the substituted pyridazine of Formula (I′) is of Formula (II′): 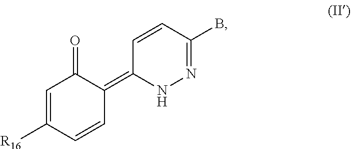
or a pharmaceutically acceptable salt thereof, wherein:
R₁₆ is a 5 member heteroaryl having one ring nitrogen atom and 0 or 1 additional ring heteroatom selected from N, O, or S, wherein the heteroaryl is optionally substituted with C₁-C₄alkyl.

23. The method of claim 22, wherein R₁₆ is thiophene, furan, pyrrole, dihydropyrrole, imidazole, pyrazole, pyrazine, isothiazole, isoxazole, triazole, tetrazole, oxazole, isoxazole, thiazole, or isothiazole.

24. The method of claim 22 or 23, wherein R₁₆ is pyrazole.

25. The method of claim 24, wherein R₁₆ is

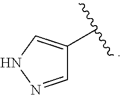 [SMILES: *c1cn[nH]c1]

26. The method of any one of claims 22-25, wherein the substituted pyridazine of Formula (II′) is of the formula: 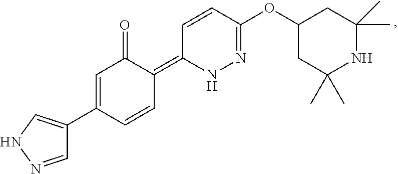 [SMILES: CC1(C)CC(OC2=NN/C(=C3\C=CC(c4cn[nH]c4)=CC3=O)C=C2)CC(C)(C)N1]
or a pharmaceutically acceptable salt thereof.

27. The method of claim 9, wherein the substituted pyridazine is a compound of Formula (III): 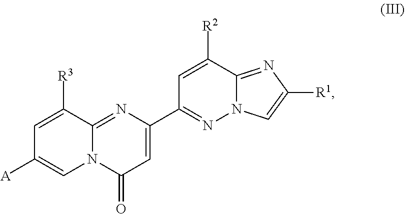
or a pharmaceutically acceptable salt thereof, wherein:
R¹ is hydrogen or C₁₋₇-alkyl;
R² is hydrogen, cyano, C₁₋₇-alkyl, C₁₋₇-haloalkyl, or C₃₋₈-cycloalkyl;
R³ is hydrogen, C₁₋₇-alkyl, or C₃₋₈-cycloalkyl;
A is N-heterocycloalkyl or NR¹²R¹³, wherein N-heterocycloalkyl comprises 1 or 2 nitrogen ring atoms and is optionally substituted with 1, 2, 3, or 4 substituents selected from R¹⁴;
R¹² is heterocycloalkyl comprising 1 nitrogen ring atom, wherein heterocycloalkyl is optionally substituted with 1, 2, 3, or 4 substituents selected from R¹⁴;
R¹³ is hydrogen, C₁₋₇-alkyl, or C₃₋₈-cycloalkyl;
R¹⁴ is independently selected from hydrogen, C₁₋₇-alkyl, amino, amino-C₁₋₇-alkyl, C₃₋₈-cycloalkyl, and heterocycloalkyl, or two R¹⁴ together form C₁₋₇-alkylene;
with the proviso that if A is N-heterocycloalkyl comprising only 1 nitrogen ring atom, then at least one R¹⁴ substituent is amino or amino-C₁₋₇-alkyl.

28. The method of claim 27, wherein the compound of Formula (III), is of the formula: 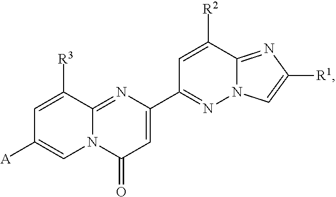
or a pharmaceutically acceptable salt thereof, wherein:
R¹ is hydrogen or C₁₋₇-alkyl;
R² is hydrogen, cyano, C₁₋₇-alkyl, C₁₋₇-haloalkyl, or C₃₋₈-cycloalkyl;
R³ is hydrogen, C₁₋₇-alkyl, or C₃₋₈-cycloalkyl;
A is N-heterocycloalkyl comprising 1 or 2 nitrogen ring atoms, wherein N-heterocycloalkyl is optionally substituted with 1, 2, 3, or 4 substituents selected from R¹⁴;
R¹⁴ is independently selected from hydrogen, C₁₋₇-alkyl, amino, amino-C₁₋₇-alkyl, C₃₋₈-cycloalkyl, and heterocycloalkyl, or two R¹⁴ together form C₁₋₇-alkylene;
with the proviso that if A is N-heterocycloalkyl comprising only 1 nitrogen ring atom, then at least one R¹⁴ substituent is amino or amino-C₁₋₇-alkyl.

29. The method of claims 27 or 28, wherein R¹ is C₁₋₇-alkyl.

30. The method of claim 29, wherein R¹ is methyl.

31. The method of any one of claims 27 to 30, wherein R² is hydrogen.

32. The method of any one of claims 27-30, wherein R² is C₁₋₇-alkyl.

33. The method of claim 32, wherein R² is methyl.

34. The method of any one of claims 27-33, wherein R³ is hydrogen.

35. The method of any one of claims 27-34, wherein R³ is C₁₋₇-alkyl.

36. The method of claim 35, wherein R³ is methyl.

37. The method of any one of claims 27-36, wherein A is N-heterocycloalkyl or NR¹²R¹³, wherein N-heterocycloalkyl comprises 1 or 2 nitrogen ring atoms and is optionally substituted with 1, 2, 3, or 4 substituents selected from R¹⁴;
R¹² is heterocycloalkyl comprising 1 nitrogen ring atom, wherein heterocycloalkyl is optionally substituted with 1, 2, 3, or 4 substituents selected from R¹⁴;
R¹³ is hydrogen, C₁₋₇-alkyl, or C₃₋₈-cycloalkyl;
R¹⁴ is independently selected from hydrogen, C₁₋₇-alkyl, amino, amino-C₁₋₇-alkyl, C₃₋₈-cycloalkyl, and heterocycloalkyl, or two R¹⁴ together form C₁₋₇-alkylene;
with the proviso that if A is N-heterocycloalkyl comprising only 1 nitrogen ring atom, then at least one R¹⁴ substituent is amino or amino-C₁₋₇-alkyl.

38. The method of claim 37, wherein R¹² is piperidinyl optionally substituted with 1, 2, 3, or 4 substituents selected from R¹⁴.

39. The method of claim 37, wherein A is of the formula: 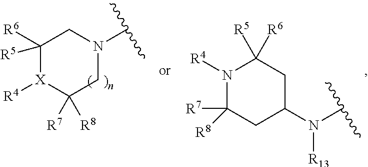
wherein:
X is N or CH;
R⁴ is hydrogen, C₁₋₇-alkyl, or —(CH₂)ₘ—NR⁹R¹⁰;
R⁵ is hydrogen or C₁₋₇-alkyl;
R⁶ is hydrogen or C₁₋₇-alkyl;
R⁷ is hydrogen or C₁₋₇-alkyl;
R⁸ is hydrogen or C₁₋₇-alkyl;
R⁹ and R¹⁰ are independently selected from hydrogen, C₁₋₇-alkyl, and C₃₋₈-cycloalkyl;
R¹³ is hydrogen, C₁₋₇-alkyl, or C₃₋₈-cycloalkyl;
n is 0, 1, or 2;
m is 0, 1, 2, or 3;
or R⁴ and R⁵ together form C₁₋₇-alkylene;
or R⁴ and R⁷ together form C₁₋₇-alkylene;
or R⁵ and R⁶ together form C₂₋₇-alkylene;
or R⁵ and R⁷ together form C₁₋₇-alkylene;
or R⁵ and R⁹ together form C₁₋₇-alkylene;
or R⁷ and R⁸ together form C₂₋₇-alkylene;
or R⁷ and R⁹ together form C₁₋₇-alkylene;
or R⁹ and R¹⁰ together form C₂₋₇-alkylene;
with the proviso that if X is CH then R⁴ is —(CH₂)ₘ—NR⁹R¹⁰; and
with the proviso that if X is N and R⁴ is —(CH₂)ₘ—NR⁹R¹⁰ then m is 2 or 3.

40. The method of claim 39, wherein A is of the formula: 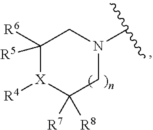
wherein:
X is N or CH;
R⁴ is hydrogen, C₁₋₇-alkyl, or —(CH₂)ₘ—NR⁹R¹⁰;
R⁵ is hydrogen or C₁₋₇-alkyl;
R⁶ is hydrogen or C₁₋₇-alkyl;
R⁷ is hydrogen or C₁₋₇-alkyl;
R⁸ is hydrogen or C₁₋₇-alkyl;
R⁹ and R¹⁰ are independently selected from hydrogen, C₁₋₇-alkyl, and C₃₋₈-cycloalkyl;
n is 0, 1, or 2;
m is 0, 1, 2, or 3;
or R⁴ and R⁵ together form C₁₋₇-alkylene;
or R⁴ and R⁷ together form C₁₋₇-alkylene;
or R⁵ and R⁶ together form C₂₋₇-alkylene;
or R⁵ and R⁷ together form C₁₋₇-alkylene;
or R⁵ and R⁹ together form C₁₋₇-alkylene;
or R⁷ and R⁸ together form C₂₋₇-alkylene;
or R⁷ and R⁹ together form C₁₋₇-alkylene;
or R⁹ and R¹⁰ together form C₂₋₇-alkylene;
with the proviso that if X is CH then R⁴ is —(CH₂)ₘ—NR⁹R¹⁰; and
with the proviso that if X is N and R⁴ is —(CH₂)ₘ—NR⁹R¹⁰ then m is 2 or 3.

41. The method of claim 40, wherein X is N.

42. The method of claim 40 or 41, wherein n is 1.

43. The method of any one of claims 40-42, wherein R⁶ is hydrogen, methyl or —(CH₂)ₘ NR⁹R¹⁰.

44. The method of claim 43, wherein R⁶ is hydrogen.

45. The method of claim 43, wherein R⁶ is methyl.

46. The method of any one of claims 40-45, wherein R⁷ is hydrogen.

47. The method of any one of claims 40-45, wherein R⁷ is methyl.

48. The method of any one of claims 40-47, wherein m is 0.

49. The method of any one of claims 40-42, wherein R⁴ and R⁵ together form propylene.

50. The method of any one of claims 40-42, wherein R⁵ and R⁶ together form ethylene.

51. The method of any one of claims 40-42, wherein R⁹ and R¹⁰ together form butylene.

52. The method of any one of claim 47, or 39-42, wherein A is

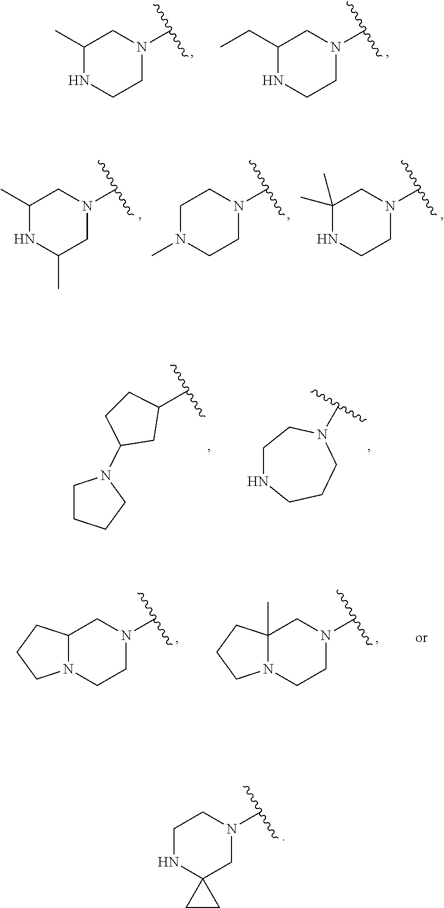

53. The method of claim 52, wherein A is

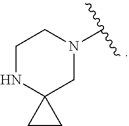 [SMILES: *N1CCNC2(CC2)C1]

54. The method of claim 53, wherein the pyridazine derivative is of the formula: 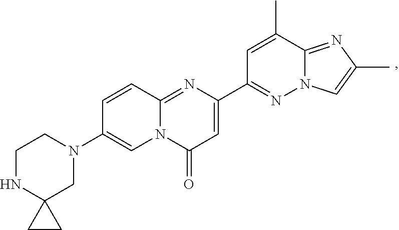 [SMILES: Cc1cn2nc(-c3cc(=O)n4cc(N5CCNC6(CC6)C5)ccc4n3)cc(C)c2n1]
or a pharmaceutically acceptable salt thereof.

55. The method of claim 9, wherein the pyridazine derivative is Risdiplam.

56. The method of claim 9, wherein the pyridazine derivative is Branaplam.

57. The method of any one of claims 1-56, wherein the ASO alters the splicing pattern of survival of motor neuron 2 (SMN2) pre-mRNA.

58. The method of claim 57, wherein the ASO promotes the inclusion of exon 7 in survival of motor neuron 2 (SMN2) mRNA.

59. The method of any one of claims 1-58, wherein the ASO comprises a nucleic acid sequence of SEQ ID NOs: 1.

60. The method of claim any one of claims 1-59, wherein the ASO is nusinersen.

61. The method of any one of claims 1-60, wherein the small molecule that increases SMN function and the rAAV are administered simultaneously.

62. The method of any one of claims 1-60, wherein the small molecule that increases SMN function and the ASO are administered simultaneously.

63. The method of any one of claims 1-60, wherein the small molecule, the rAAV, and the ASO are administered simultaneously.

64. The method of any one of claims 1-60, wherein the small molecule that increases SMN function and the rAAV are administered concurrently.

65. The method of any one of claims 1-60, wherein the small molecule that increases SMN function and the ASO are administered concurrently.

66. The method of any one of claims 1-60, wherein the small molecule that increases SMN function, the rAAV, and the ASO are administered concurrently.

67. The method of any one of claims 1-60, wherein the small molecule that increases SMN function and the rAAV are administered sequentially.

68. The method of any one of claims 1-60, wherein the small molecule that increases SMN function and the ASO are administered sequentially.

69. The method of any one of claims 1-60, wherein the small molecule that increases SMN function, the rAAV, and the ASO are administered sequentially.

70. A method of treating spinal muscular atrophy (SMA) in a subject having SMA, the method comprising administering an effective amount of a composition comprising an ASO that increases full-length SMN2 mRNA to a subject that was previously administered a small molecule that increases SMN function.

71. A method of treating spinal muscular atrophy (SMA) in a subject having SMA, the method comprising administering an effective amount of a composition comprising an rAAV encoding SMN1 to a subject that was previously administered a small molecule that increases SMN function.


SEQUENCE LISTING (26 sequences)

SEQ ID NO: 1 - DNA, 18 bp, Artificial Sequence
  Synthetic polynucleotide
tcactttcataatgctgg

SEQ ID NO: 2 - DNA, 15 bp, Artificial Sequence
  Synthetic polynucleotide
tgctggcagacttac

SEQ ID NO: 3 - DNA, 15 bp, Artificial Sequence
  Synthetic polynucleotide
cataatgctggcaga

SEQ ID NO: 4 - DNA, 15 bp, Artificial Sequence
  Synthetic polynucleotide
tcataatgctggcag

SEQ ID NO: 5 - DNA, 15 bp, Artificial Sequence
  Synthetic polynucleotide
ttcataatgctggca

SEQ ID NO: 6 - DNA, 15 bp, Artificial Sequence
  Synthetic polynucleotide
tttcataatgctggc

SEQ ID NO: 7 - DNA, 20 bp, Artificial Sequence
  Synthetic polynucleotide
attcactttcataatgctgg

SEQ ID NO: 8 - DNA, 15 bp, Artificial Sequence
  Synthetic polynucleotide
ctttcataatgctgg

SEQ ID NO: 9 - DNA, 12 bp, Artificial Sequence
  Synthetic polynucleotide
tcataatgctgg

SEQ ID NO: 10 - DNA, 15 bp, Artificial Sequence
  Synthetic polynucleotide
actttcataatgctg

SEQ ID NO: 11 - DNA, 12 bp, Artificial Sequence
  Synthetic polynucleotide
ttcataatgctg

SEQ ID NO: 12 - DNA, 15 bp, Artificial Sequence
  Synthetic polynucleotide
cactttcataatgct

SEQ ID NO: 13 - DNA, 12 bp, Artificial Sequence
  Synthetic polynucleotide
tttcataatgct

SEQ ID NO: 14 - DNA, 15 bp, Artificial Sequence
  Synthetic polynucleotide
tcactttcataatgc

SEQ ID NO: 15 - DNA, 12 bp, Artificial Sequence
  Synthetic polynucleotide
ctttcataatgc

SEQ ID NO: 16 - DNA, 15 bp, Artificial Sequence
  Synthetic polynucleotide
ttcactttcataatg

SEQ ID NO: 17 - DNA, 12 bp, Artificial Sequence
  Synthetic polynucleotide
actttcataatg

SEQ ID NO: 18 - DNA, 15 bp, Artificial Sequence
  Synthetic polynucleotide
attcactttcataat

SEQ ID NO: 19 - DNA, 12 bp, Artificial Sequence
  Synthetic polynucleotide
cactttcataat

SEQ ID NO: 20 - DNA, 15 bp, Artificial Sequence
  Synthetic polynucleotide
gattcactttcataa

SEQ ID NO: 21 - DNA, 12 bp, Artificial Sequence
  Synthetic polynucleotide
tcactttcataa

SEQ ID NO: 22 - DNA, 12 bp, Artificial Sequence
  Synthetic polynucleotide
ttcactttcata

SEQ ID NO: 23 - DNA, 12 bp, Artificial Sequence
  Synthetic polynucleotide
attcactttcat

SEQ ID NO: 24 - DNA, 15 bp, Artificial Sequence
  Synthetic polynucleotide
agtaagattcacttt

SEQ ID NO: 25 - RNA, 18 nt, Artificial Sequence
  Synthetic polynucleotide
  misc_feature (1)..(1): 2′-O-(2-methoxyethyl)-5-methyl-P-thiouridine
  misc_feature (2)..(2): 2′-O-(2-methoxyethyl)-5-methyl-P-thiocytidine
  misc_feature (3)..(3): 2′-O-(2-methoxyethyl)-P-thioadenine
  misc_feature (4)..(4): 2′-O-(2-methoxyethyl)-5-methyl-P-thiocytidine
  misc_feature (5)..(5): 2′-O-(2-methoxyethyl)-5-methyl-P-thiouridine
  misc_feature (6)..(6): 2′-O-(2-methoxyethyl)-5-methyl-P-thiouridine
  misc_feature (7)..(7): 2′-O-(2-methoxyethyl)-5-methyl-P-thiouridine
  misc_feature (8)..(8): 2′-O-(2-methoxyethyl)-5-methyl-P-thiocytidine
  misc_feature (9)..(9): 2′-O-(2-methoxyethyl)-P-thioadenine
  misc_feature (10)..(10): 2′-O-(2-methoxyethyl)-5-methyl-P-thiouridine
  misc_feature (11)..(11): 2′-O-(2-methoxyethyl)-P-thioadenine
  misc_feature (12)..(12): 2′-O-(2-methoxyethyl)-P-thioadenine
  misc_feature (13)..(13): 2′-O-(2-methoxyethyl)-5-methyl-P-thiouridine
  misc_feature (14)..(14): 2′-O-(2-methoxyethyl)-P-thioguanine
  misc_feature (15)..(15): 2′-O-(2-methoxyethyl)-5-methyl-P-thiocytidine
  misc_feature (16)..(16): 2′-O-(2-methoxyethyl)-5-methyl-P-thiouridine
  misc_feature (17)..(17): 2′-O-(2-methoxyethyl)-P-thioguanine
  misc_feature (18)..(18): 2′-O-(2-methoxyethyl)-P-thioguanine
ucacuuucauaaugcugg

SEQ ID NO: 26 - RNA, 18 nt, Artificial Sequence
  Synthetic polynucleotide
ucacuuucauaaugcugg